SEQUENCE LISTING (110 sequences)

SEQ ID NO: 1 - RNA, 23 nt, Artificial sequence
  Single strand DNA oligonucleotide
aacauucaacgcugucggugagu

SEQ ID NO: 2 - RNA, 23 nt, Artificial sequence
  Single strand DNA oligonucleotide
acucaccgacagcguugaauguu

SEQ ID NO: 3 - protein, 26 aa, Artificial sequence
  ARP peptide (synthetic)
GMQGPAGSGWEEGSGSPPGVTPLFSP

SEQ ID NO: 4 - protein, 40 aa, Artificial sequence
  ASP peptide (synthetic)
DTLDEAERQWKAEFHRWSSYMVHWKNQFDHYSKQDRCSDL

SEQ ID NO: 5 - DNA, 20 bp, Artificial sequence
  Single strand DNA oligonucleotide
ctgccacgttctcctgcacc

SEQ ID NO: 6 - DNA, 21 bp, Artificial sequence
  Single strand DNA oligonucleotide
ggtacagtcaacggtcagtgg

SEQ ID NO: 7 - DNA, 22 bp, Artificial sequence
  Single strand DNA oligonucleotide
ggactccaaggaacattcaacg

SEQ ID NO: 8 - DNA, 34 bp, Artificial sequence
  Single strand DNA oligonucleotide
tgaaacaacatacaattccatcatgaagtgtgac

SEQ ID NO: 9 - DNA, 30 bp, Artificial sequence
  Single strand DNA oligonucleotide
aggagcgataatcttgatcttcatggtgct

SEQ ID NO: 10 - RNA, 50 nt, Artificial sequence
  DIG labeled RNA porbe
ccgggggacgucggggugggguggggaugggcagagucuggggcucgucu

SEQ ID NO: 11 - RNA, 50 nt, Artificial sequence; identical to SEQ ID NO: 10
  2-O-methylated cRNA probe

SEQ ID NO: 12 - DNA, 20 bp, Artificial sequence
  Synthetic oligonucleotide containing unmethyatde CpG motives
gggggacgatcgtcgggggg

SEQ ID NO: 13 - RNA, 110 nt, Artificial sequence
  Synthetic MiRNA-181a precursor
agaagggcuaucaggccagccuucagaggacuccaaggaacauucaacgcugucggugaguuugggauuugaaaaaaccacugaccguugacuguaccuugggguccuua

SEQ ID NO: 14 - DNA, 71 bp, Artificial sequence
  MiRNA-181AA Amplicon
ggactccaaggaacattcaacgctgtcggtgagtttgggatttgaaaaaaccactgaccgttgactgtacc

SEQ ID NO: 15 - DNA, 2156 bp, Homo sapiens
cagcctgcgccggggaacatcggccgcctccagctcccggcgcggcccggcccggcccggctcggccgcctcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctggcatgtcttcaggctgagctcctccccgcgtgccttcgccctctggctgcaaataaactgttacaggcc

SEQ ID NO: 16 - DNA, 2990 bp, Homo sapiens
cagcctgcgccggggaacatcggccgcctccagctcccggcgcggcccggcccggcccggctcggccgcctcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccggtatgcaggggccagcgggcagcggctgggaggaggggagtgggagcccgccaggtgtaacccctctcttctccccctagccctcggaggctcccagcacctgcccaggcttcacccatggggaggctgctccgaggcccggcctccccctgcccctcctcctcctccaccagcttctcctcctcttcctctcccacctccggcggctgtgaacacggcctcttcccctacggccacaggggcccctcctctaatgagtggtcggaccgtggggaagggccccactcagggatctcagacctagtgctcccttcctcctcaaaccgagagactcacactggacagggcaggaggagggggccgtgcctcccacccttctcagggacccccacgcctttgttgtttgaatggaaatggaaaagccagtattcttttataaaattatcttttggaacctgagcctgacattggggggaagtgggaggccccggacggggtagcaccccccattggggctataacggtcaaccatttctgtctcttctttttcccccaacctccccctcctgtcccctctgttcccgtcttccggtcattcttttctcctcctctctccttcctgctgtccttctccggccccgcctctgccctcatcctccctctcgtctttcgcacattctcctgatcctcttgccaccgtcccacgtggtcgcctgcatttctccgtgcgtcctccctgcactgaaacccccccttcaacccgcccaaatgtccgatccccgaccttcctcgtgccgtcctcccctcccgcctcgctgggcgccctggccgcagacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctggcatgtcttcaggctgagctcctccccgcgtgccttcgccctctggctgcaaataaactgttacaggcc

SEQ ID NO: 17 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 18 - protein, 600 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATGMQGPAGSGWEEGSGSPPGVTPLFSP

SEQ ID NO: 19 - DNA, 24 bp, Artificial sequence
  Synthetic oligonucleotide containing unmethyatde CpG motives
tcgtcgttttgtcgttttgtcgtt

SEQ ID NO: 20 - RNA, 20 nt, Artificial sequence
  An inverse sequence oligonucleotide
ccacguccucuugcaccguc

SEQ ID NO: 21 - RNA, 23 nt, Homo sapiens
  misc_feature: Naturally occurring endogenous Mature MiRNA
aacauucaacgcugucggugagu

SEQ ID NO: 22 - RNA, 110 nt, Homo sapiens; identical to SEQ ID NO: 13
  misc_feature: Naturaly occurring MiRNA-181

SEQ ID NO: 23 - RNA, 23 nt, Artificial sequence
  Oxymethylated synthetic RNA oligonucleotide
aacauucaacgcugucggugagu

SEQ ID NO: 24 - RNA, 23 nt, Artificial sequence
  Oxymethylated synthetic RNA oligonucleotide
acucaccgacagcguugaauguu

SEQ ID NO: 25 - RNA, 23 nt, Artificial sequence
  MiRNA oligonucleotide
uagcaccauuugaaaucaguguu

SEQ ID NO: 26 - RNA, 21 nt, Artificial sequence
  MiRNA oligonucleotide
uacucaguaaggcauuguucu

SEQ ID NO: 27 - RNA, 22 nt, Artificial sequence
  MiRNA oligonucleotide
agugccgcagaguuuguagugu

SEQ ID NO: 28 - RNA, 22 nt, Artificial sequence
  MiRNA oligonucleotide
uguaaacauccucgacuggaag

SEQ ID NO: 29 - RNA, 20 nt, Artificial sequence
  MiRNA oligonucleotide
acugcagugaaggcacuugu

SEQ ID NO: 30 - RNA, 22 nt, Artificial sequence
  MiRNA oligonucleotide
caucaaaguggaggcccucucu

SEQ ID NO: 31 - RNA, 22 nt, Artificial sequence
  MiRNA oligonucleotide
ggcagaggagggcuguucuucc

SEQ ID NO: 32 - RNA, 22 nt, Artificial sequence
  MiRNA oligonucleotide
aaagugcuucccuuuugugugu

SEQ ID NO: 33 - RNA, 24 nt, Artificial sequence
  MiRNA oligonucleotide
caaagugcuuacagugcagguagu

SEQ ID NO: 34 - RNA, 22 nt, Artificial sequence
  MiRNA oligonucleotide
cuuucagucggauguuugcagc

SEQ ID NO: 35 - RNA, 23 nt, Artificial sequence
  MiRNA oligonucleotide
cagugcaauaguauugucaaagc

SEQ ID NO: 36 - RNA, 23 nt, Artificial sequence
  MiRNA oligonucleotide
aagugccgccagguuuugagugu

SEQ ID NO: 37 - RNA, 22 nt, Artificial sequence
  MiRNA oligonucleotide
ugagaacugaauuccauggguu

SEQ ID NO: 38 - RNA, 20 nt, Artificial sequence
  MiRNA oligonucleotide
auuccuagaaauuguucaua

SEQ ID NO: 39 - RNA, 22 nt, Artificial sequence
  MiRNA oligonucleotide
cuggacuuagggucagaaggcc

SEQ ID NO: 40 - RNA, 22 nt, Artificial sequence
  MiRNA oligonucleotide
agagguauagggcaugggaaaa

SEQ ID NO: 41 - RNA, 22 nt, Artificial sequence
  MiRNA oligonucleotide
uauacaagggcaagcucucugu

SEQ ID NO: 42 - RNA, 22 nt, Artificial sequence
  MiRNA oligonucleotide
uagcagcacguaaauauuggcg

SEQ ID NO: 43 - RNA, 24 nt, Artificial sequence
  MiRNA oligonucleotide
uacugcaucaggaacugauuggau

SEQ ID NO: 44 - RNA, 22 nt, Artificial sequence
  MiRNA oligonucleotide
uuaucagaaucuccagggguac

SEQ ID NO: 45 - RNA, 23 nt, Artificial sequence
  MiRNA oligonucleotide
uaagugcuuccauguuuugguga

SEQ ID NO: 46 - RNA, 23 nt, Artificial sequence
  MiRNA oligonucleotide
uauggcacugguagaauucacug

SEQ ID NO: 47 - RNA, 21 nt, Artificial sequence
  MiRNA oligonucleotide
uggaauguaaagaaguaugua

SEQ ID NO: 48 - RNA, 23 nt, Artificial sequence
  MiRNA oligonucleotide
uaagugcuuccauguuucagugg

SEQ ID NO: 49 - RNA, 23 nt, Artificial sequence
  MiRNA oligonucleotide
ugugcaaaucuaugcaaaacuga

SEQ ID NO: 50 - RNA, 23 nt, Artificial sequence
  MiRNA oligonucleotide
uaagugcuuccauguuuuaguag

SEQ ID NO: 51 - RNA, 22 nt, Artificial sequence
  MiRNA oligonucleotide
uagguuauccguguugccuucg

SEQ ID NO: 52 - RNA, 24 nt, Artificial sequence
  MiRNA oligonucleotide
aaaagugcuuacagugcagguagc

SEQ ID NO: 53 - RNA, 22 nt, Artificial sequence
  MiRNA oligonucleotide
uaugcaagggcaagcucucuuc

SEQ ID NO: 54 - RNA, 22 nt, Artificial sequence
  MiRNA oligonucleotide
uaacagucuacagccauggucg

SEQ ID NO: 55 - RNA, 22 nt, Artificial sequence
  MiRNA oligonucleotide
ucacagugaaccggucucuuuu

SEQ ID NO: 56 - RNA, 23 nt, Artificial sequence
  MiRNA oligonucleotide
uccgucucaguuacuuuauagcc

SEQ ID NO: 57 - RNA, 22 nt, Artificial sequence
  MiRNA oligonucleotide
agugccgcagaguuuguagugu

SEQ ID NO: 58 - RNA, 21 nt, Artificial sequence
  MiRNA oligonucleotide
cuuuuugcggucugggcuugc

SEQ ID NO: 59 - RNA, 22 nt, Artificial sequence
  MiRNA oligonucleotide
agcucggucugaggccccucag

SEQ ID NO: 60 - RNA, 22 nt, Artificial sequence
  MiRNA oligonucleotide
gaaguuguucgugguggauucg

SEQ ID NO: 61 - RNA, 22 nt, Artificial sequence
  MiRNA oligonucleotide
uugguccccuucaaccagcugu

SEQ ID NO: 62 - RNA, 23 nt, Artificial sequence
  MiRNA oligonucleotide
aacacgguccacuaacccucagu

SEQ ID NO: 63 - RNA, 23 nt, Artificial sequence
  MiRNA oligonucleotide
cccaguguucagacuaccuguuc

SEQ ID NO: 64 - RNA, 23 nt, Artificial sequence
  MiRNA oligonucleotide
gcaaagcacacggccugcagaga

SEQ ID NO: 65 - RNA, 21 nt, Artificial sequence
  MiRNA oligonucleotide
uucacaguggcuaaguuccgc

SEQ ID NO: 66 - RNA, 21 nt, Artificial sequence
  MiRNA oligonucleotide
aauauaacacagauggccugu

SEQ ID NO: 67 - RNA, 22 nt, Artificial sequence
  MiRNA oligonucleotide
uucaacggguauuuauugagca

SEQ ID NO: 68 - RNA, 22 nt, Artificial sequence
  MiRNA oligonucleotide
ucagugcacuacagaacuuugu

SEQ ID NO: 69 - RNA, 22 nt, Artificial sequence
  MiRNA oligonucleotide
aaagugcuguucgugcagguag

SEQ ID NO: 70 - RNA, 18 nt, Artificial sequence
  MiRNA oligonucleotide
uggagagaaaggcaguuc

SEQ ID NO: 71 - RNA, 24 nt, Artificial sequence
  MiRNA oligonucleotide
caaagugcuuacagugcagguagu

SEQ ID NO: 72 - RNA, 19 nt, Artificial sequence
  MiRNA oligonucleotide
gugcauuguaguugcauug

SEQ ID NO: 73 - RNA, 23 nt, Artificial sequence
  MiRNA oligonucleotide
ucuuugguuaucuagcuguauga

SEQ ID NO: 74 - RNA, 21 nt, Artificial sequence
  MiRNA oligonucleotide
uaaagcuagauaaccgaaagu

SEQ ID NO: 75 - RNA, 21 nt, Artificial sequence
  MiRNA oligonucleotide
ugauuguccaaacgcaauucu

SEQ ID NO: 76 - RNA, 23 nt, Artificial sequence
  MiRNA oligonucleotide
cagugcaauaguauugucaaagc

SEQ ID NO: 77 - RNA, 22 nt, Artificial sequence
  MiRNA oligonucleotide
agcuacauugucugcuggguuu

SEQ ID NO: 78 - RNA, 24 nt, Artificial sequence
  MiRNA oligonucleotide
guccaguuuucccaggaaucccuu

SEQ ID NO: 79 - RNA, 23 nt, Artificial sequence
  MiRNA oligonucleotide
uggagugugacaaugguguuugu

SEQ ID NO: 80 - RNA, 22 nt, Artificial sequence
  MiRNA oligonucleotide
cagugguuuuacccuaugguag

SEQ ID NO: 81 - RNA, 21 nt, Artificial sequence
  MiRNA oligonucleotide
uucaaguaauccaggauaggc

SEQ ID NO: 82 - RNA, 21 nt, Artificial sequence
  MiRNA oligonucleotide
uagcagcacagaaauauuggc

SEQ ID NO: 83 - RNA, 22 nt, Artificial sequence
  MiRNA oligonucleotide
aucauagaggaacauccacuuu

SEQ ID NO: 84 - RNA, 21 nt, Artificial sequence
  MiRNA oligonucleotide
augaccuaugaauugacagac

SEQ ID NO: 85 - RNA, 20 nt, Artificial sequence
  MiRNA oligonucleotide
guguguggaaaugcuucugc

SEQ ID NO: 86 - RNA, 23 nt, Artificial sequence
  MiRNA oligonucleotide
aaagugcugcgacauuugagcgu

SEQ ID NO: 87 - RNA, 23 nt, Artificial sequence
  MiRNA oligonucleotide
ucaagagcaauaacgaaaaaugu

SEQ ID NO: 88 - RNA, 20 nt, Artificial sequence
  MiRNA oligonucleotide
uugcauagucacaaaaguga

SEQ ID NO: 89 - RNA, 21 nt, Artificial sequence
  MiRNA oligonucleotide
aucgggaaugucguguccgcc

SEQ ID NO: 90 - RNA, 22 nt, Artificial sequence
  MiRNA oligonucleotide
uggcucaguucagcaggaacag

SEQ ID NO: 91 - RNA, 22 nt, Artificial sequence
  MiRNA oligonucleotide
cagugcaaugaugaaagggcau

SEQ ID NO: 92 - RNA, 22 nt, Artificial sequence
  MiRNA oligonucleotide
uuaaugcuaaucgugauagggg

SEQ ID NO: 93 - RNA, 21 nt, Artificial sequence
  MiRNA oligonucleotide
ugguucuagacuugccaacua

SEQ ID NO: 94 - RNA, 21 nt, Artificial sequence
  MiRNA oligonucleotide
uaacagucuccagucacggcc

SEQ ID NO: 95 - RNA, 21 nt, Artificial sequence
  MiRNA oligonucleotide
uauugcacauuacuaaguugc

SEQ ID NO: 96 - RNA, 21 nt, Artificial sequence
  MiRNA oligonucleotide
acagcaggcacagacaggcag

SEQ ID NO: 97 - RNA, 22 nt, Artificial sequence
  MiRNA oligonucleotide
gugaaauguuuaggaccacuag

SEQ ID NO: 98 - RNA, 22 nt, Artificial sequence
  MiRNA oligonucleotide
aaggagcucacagucuauugag

SEQ ID NO: 99 - RNA, 23 nt, Artificial sequence
  MiRNA oligonucleotide
ucccugagacccuuuaaccugug

SEQ ID NO: 100 - RNA, 22 nt, Artificial sequence
  MiRNA oligonucleotide
ucccugagacccuaacuuguga

SEQ ID NO: 101 - DNA, 23 bp, Artificial sequence
  Single strand DNA oligonucleotide
aaattactgaagcccacttggtt

SEQ ID NO: 102 - DNA, 21 bp, Artificial sequence
  Single strand DNA oligonucleotide
actctgcaagatgccacaagg

SEQ ID NO: 103 - DNA, 24 bp, Artificial sequence
  Single strand DNA oligonucleotide
tagtccttcctaaccccaatttcc

SEQ ID NO: 104 - DNA, 21 bp, Artificial sequence
  Single strand DNA oligonucleotide
ttggtccttagccactccttc

SEQ ID NO: 105 - DNA, 22 bp, Artificial sequence
  Single strand DNA oligonucleotide
atgagcactgaaagcatgatcc

SEQ ID NO: 106 - DNA, 22 bp, Artificial sequence
  Single strand DNA oligonucleotide
gagggctgattagagagaggtc

SEQ ID NO: 107 - DNA, 23 bp, Artificial sequence
  Synthetic miR mimetic oligonucleotide
  misc_feature (3)..(): LMA modified residue
  misc_feature (6)..(): LMA modified residue
  misc_feature (9)..(): LMA modified residue
  misc_feature (12)..(): LMA modified residue
  misc_feature (15)..(): LMA modified residue
  misc_feature (18)..(): LMA modified residue
  misc_feature (21)..(): LMA modified residue
taacagtctacagccatggtctg

SEQ ID NO: 108 - DNA, 24 bp, Artificial sequence
  Synthetic miR mimetic oligonucleotide
  misc_feature (3)..(): LMA modified residue
  misc_feature (6)..(): LMA modified residue
  misc_feature (9)..(): LMA modified residue
  misc_feature (12)..(): LMA modified residue
  misc_feature (15)..(): LMA modified residue
  misc_feature (18)..(): LMA modified residue
  misc_feature (21)..(): LMA modified residue
  misc_feature (24)..(): LMA modified residue
tggttctatacgacttgccaacta

SEQ ID NO: 109 - DNA, 22 bp, Artificial sequence
  Synthetic miR mimetic oligonucleotide
  misc_feature (3)..(): LMA modified residue
  misc_feature (6)..(): LMA modified residue
  misc_feature (9)..(): LMA modified residue
  misc_feature (12)..(): LMA modified residue
  misc_feature (15)..(): LMA modified residue
  misc_feature (18)..(): LMA modified residue
  misc_feature (21)..(): LMA modified residue
cgaccatggctgtagactgtta

SEQ ID NO: 110 - DNA, 21 bp, Artificial sequence
  Synthetic miR mimetic oligonucleotide
  misc_feature (2)..(): LMA modified residue
  misc_feature (5)..(): LMA modified residue
  misc_feature (8)..(): LMA modified residue
  misc_feature (11)..(): LMA modified residue
  misc_feature (14)..(): LMA modified residue
  misc_feature (17)..(): LMA modified residue
  misc_feature (20)..(): LMA modified residue
tagttggcaagtctagaacca

(19) United States
(12) Patent Application Publication
(10) Pub. No.: US 2007/0293452 A1
(43) Pub. Date: Dec. 20, 2007

(54) Agents, compositions and methods for treating pathologies in which regulating an ache-associated biological pathway is beneficial

(75) Inventors: Hermona Soreq, Jerusalem (IL); Iftach Shaked, Jerusalem (IL); Ran Avni, Jerusalem (IL); Ari Meerson, Givat Zeev (IL)

Correspondence Address: Martin D. Moynihan;PRTSI, Inc., P.O. Box 16446, Arlington, VA, 22215, US

(73) Assignee: Yissum Research Development Company of the Hebrew University of Jerusalem, Jerusalem (IL)

(21) Appl. No.: 11/808,212

(22) Filed: Jun. 7, 2007

Related U.S. Application Data
(63) Continuation-in-part of application No. 11/714,861, filed on Mar. 7, 2007.
Application No. 11/714,861 is a continuation-in-part of application No. PCT/IL05/00945, filed on Sep. 7, 2005.
(60) Provisional application No. 60/671,452, filed on Apr. 15, 2005.
(60) Provisional application No. 60/607,254, filed on Sep. 7, 2004.

(51) Int. Cl. C12N 5/08; A61K 31/7052; A61P 43/00; C12Q 1/68; C07H 19/00; C12N 15/00

(52) U.S. Cl. 514/44; 435/375; 435/377; 435/455; 435/6; 536/22.1

(57) ABSTRACT

The present invention provides agents which are capable of regulating the function of a micro-RNA component which can be used to regulate an ACHE-associated biological pathway. In addition, the present invention provides methods and pharmaceutical compositions for the treatment of various pathologies related to AChE-associated biological pathways such as apoptosis, aberrant cholinergic signaling, abnormal hematopoietic proliferation and/or differentiation, cellular stress, exposure to inflammatory response-inducing agents, and/or exposure to organophosphates or other AChE inhibitors.

### RELATED APPLICATIONS

This application is a continuation in part of U.S. patent application Ser. No. 11/714,861 filed on Mar. 7, 2007, which is a continuation-in-part of PCT International Patent Application No. PCT/IL2005/000945 filed on Sep. 7, 2005, which claims the benefit of U.S. Provisional Patent Application Nos. 60/671,452 filed on Apr. 15, 2005; 60/607,254 filed on Sep. 7, 2004. The contents of the above Applications are all incorporated herein by reference.

### FIELD AND BACKGROUND OF THE INVENTION

The present invention relates to isolated polynucleotides, pharmaceutical compositions containing same and methods of using same for treating a myriad of pathologies in which regulating an AChE-associated biological pathway is beneficial.

Signal transduction cascades are responsible for all functions needed for cells to maintain homeostasis, in particular intracellular responses to extracellular signals, such as hormones and neurotransmitters. At the organismal level, the systemic effects of numerous drugs and environmental agents are a result of cholinergic signaling mechanisms. Cellular signal transduction is responsible for processes such as cell differentiation, apoptosis, growth, and immune responses. The goal of therapeutic interventions for the majority of human diseases which involve defects in cellular signaling, is the targeting of the molecules involved in these mechanisms.

Cell differentiation is fine-tuned by the process of apoptosis, the elimination of nonfaisant or malfaisant cells. Apoptosis is characterized by cell shrinkage, nuclear condensation, and oligonucleosomal DNA fragmentation. The utility of this elimination is inferred from the complex series of events that recruits interleukins and cysteine-aspartate proteases (caspases) into a programmed sequence of protein degradations, culminating in cell death and the disposal of the defunct cells (Budihardjo et al., 1999; Green and Reed, 1998). Furthermore, this elaborate program is designed to eliminate cells that have been targeted as part of an integrated developmental scheme (Linette and Korsmeyer, 1994).

The apoptotic response is intrinsic to all cells of multicellular animals. There are two pathways of cell death: the so-called “death receptor pathway” and the “intrinsic pathway.” In the latter, which is activated by growth factor deprivation, glucocorticoids, or DNA damage, members of the Bcl-2 family of proteins both negatively and positively regulate apoptosis (Adams and Cory, 1998). In brief, the mitochondria release cytochrome c through the permeability transition pore (PTP) upon receiving the appropriate signal, a cleaved protein ligand called Bid. Subsequently, the initiator caspase, procaspase-9, forms the apoptosome with Apaf-1 and cytochrome c, and self-cleaves into its active form, caspase-9. Activated caspase-9 further cleaves the executioner caspase, caspase-3, from its precursor, which then cleaves cellular substrates which have been “marked” for death (FIG. 13).

Caspase-mediated pathways are activated by mitochondria in an indirect response to the release of sequestered calcium from the endoplasmic reticulum (ER). Thus, a variety of toxic insults can result in ER stress, changes in intracellular calcium (Ca²⁺) levels and ultimately lead to apoptosis and cell death (Rao et al., 2001).

Hematopoiesis is the process of differentiation of the blood cells which takes place in the bone marrow and lymphatic tissues in an adult human. The production of differentiated blood cells must be balanced by the self-renewal of hematopoietic stem cells to ensure long-term hematopoiesis throughout the individual's lifetime. Apoptosis also plays a role in regulating this hematopoietic homeostasis. There have been thus far characterized five hematopoietic differentiation pathways, all stemming from pluripotential stem cells. One of these pathways, called megakaryocytopoiesis, the maturation of platelet-forming megakaryocytes, involves the proliferation of the progenitor stem cells into myeloid and then promegakaryocytic stem cells, followed by their differentiation into megakaryocytes.

Platelet formation is the consequence of caspase activation within mature megakaryocytes, as was shown by the compartmentalization of activated caspase-3 in the pro-platelet formation territories, contrasting with the diffuse caspase localization observed during cell death (deBotton et al., 2002). Studies performed by the present inventor have also shown that megakaryocytopoiesis involves modulation of cholinergic signaling (Patinkin et al., 1990; Soreq et al., 1994; Pick et al., 2004, Blood-cell Specific Acetylcholinesterase Splice Variations under Changing Stimuli. Annals of New York Academy of Science. 1018:85-95).

Cholinergic signaling involves the release of the neurotransmitter acetylcholine by the presynaptic neuron at a chemical synapse, and the reception of signal by the postsynaptic cell. The response elicited by a neurotransmitter, whether excitatory or inhibitory, is determined by the type of postsynaptic cell receptor to which it binds. Termination of the cholinergic signal is effected by acetylcholinesterase (AChE).

Much of the proposed mechanism of regulation discussed herein is focused on destabilizing mRNA. The value of such a mechanism lies in the fact that destabilization of mRNA may contribute to target-specific therapeutic strategies for the treatment of cancer, cardiovascular disease, and other disorders or conditions (Gewirtz, 2000). This concept is attractive because mRNA is, theoretically, accessible to attack at any stage during transcription, transportation from the nucleus, and translation (Opalinska and Gewirtz, 2002). Additionally, nucleic acid therapeutics, as described below, is believed to be both highly specific and less toxic than other pharmaceutical strategies.

Destabilization, degradation or blocking of RNA translation can be mediated using four principle approaches.

One approach employs oligonucleotide aptamers as alternate binding sites, or “decoys,” for proteins that act as transcriptional activators, or as stabilizing elements that normally interact with a given mRNA (Beelman and Parker, 1995; Liebhaber, 1997). By attracting away the desired protein, the decoy may prevent transcription or induce instability, and ultimately destruction, of the mRNA (Thisted et al., 2001; Wang et al., 1995; Weiss and Liebhaber, 1995).

A second and more widely applied method of destabilizing mRNA is the “antisense” strategy, using ribozymes, DNAzymes, antisense RNA, or antisense DNA (AS-ODN). This approach to gene silencing has been the subject of numerous authoritative reviews (Gewirtz et al., 1998; Scanlon et al., 1995; Stein, 1998); in short, delivering AS-ODN into a cell where the gene of interest is expressed should lead to hybridization between the antisense sequence and the targeted gene's mRNA. Stable mRNA-antisense duplexes cannot be translated, and, depending on the chemical composition of the antisense molecule, may lead to the destruction of the mRNA by binding of endogenous nucleases, such as RNase H, or by intrinsic enzymatic activity engineered into the sequence (i.e., ribozymes and DNAzymes).

A third approach currently being developed for targeting and destabilizing mRNA is called RNA interference (RNAi) (Nishikura, 2001; Sharp, 1999). RNAi is the process by which double-stranded RNA (dsRNA) targets mRNA for destruction in a sequence-dependent manner. The mechanism of RNAi involves processing of dsRNA into approximately 21- to 23-basepair (bp) fragments that hybridize with target mRNAs and initiate their destruction. The mechanism for RNAi is fast being elucidated, although many intriguing questions remain to be answered (Nishikura, 2001). At this time, it appears likely that dsRNA is processed by an enzyme called Dicer (Hutvagner et al., 2001; Ketting et al., 2001; Nicholson and Nicholson, 2002) into 21- to 23-nt double-strands. These small cleavage products are then incorporated into the ribonucleoprotein (RNP) RNA-induced silencing complex (RISC), which scans the complementary mRNA sequence for homology to the small, now unwound, dsRNA fragment and promotes destruction of the mRNA by an enzyme integral to the complex (Hammond et al., 2001; Martinez et al., 2002; Williams and Rubin, 2002; FIGS. 1a-b).

RNAi has been successfully employed for gene silencing in a variety of experimental systems. The use of long dsRNA to silence expression in mammalian cells has been tried, initially without success (Yang et al., 2001). It has been suggested that mammalian cells recognize these sequences as invading pathogens, triggering an interferon response that leads to apoptosis and cell death (Bernstein et al., 2001). However, a number of recent reports suggest that these double-stranded RNA fragments of 21-23 nts, called short interfering RNA (siRNA), may be able to silence expression in mammalian somatic cells if appropriately modified to contain 3′-hydroxy and 5′-phosphate groups (Elbashir et al., 2001; Hannon, 2002; Yang et al., 2000; Zamore et al., 2000). While reports on the utility of this method for silencing mammalian genes continue to accumulate (Donze and Picard, 2002; Paddison et al., 2002; Sui et al., 2002; Yu et al., 2002), the successful application of this method to all types of mammalian cells remains uncertain (Yang et al., 2001), as is also true of traditional antisense experiments. Not surprisingly, the possibility of experimental artifacts being misinterpreted as specific gene targeting is being increasingly recognized (Jackson et al., 2003; Lassus et al., 2002). Accordingly, it is highly likely that many technical issues related to employing nucleic acid therapeutics in general will also apply to siRNA, including the need to deliver these molecules into cells in a bioavailable form, as well as to be able to identify accessible sequences of mRNA in a predictable manner (Holen et al., 2002).

Micro-RNAs (also known as miRNAs) are 20- to 24-nucleotide (nt) RNA molecule members of the family of non-coding small RNAs. Micro-RNAs were identified in mammals, worms, fruit flies and plants and are believed to regulate the stability of their target messenger RNA (mRNA) transcripts in a tissue- and cell type-specific manner. Principally, micro-RNAs regulate RNA stability by either binding to the 3′-untranslated region (3′-UTR) of target mRNAs and thereby suppressing translation, or in similar manner to siRNAs, binding to and destroying target transcripts in a sequence-dependent manner.

Micro-RNAs were found to be involved in various cell differentiation pathways. For example, miR-181, was found to be preferentially expressed in the B-lymphoid cells and its ectopic expression in hematopoietic stem/progenitor cells led to an increased fraction of B-lineage cells in vitro and in vivo (Chen C Z, et al., 2004). In addition, miR-23 was shown to be present in differentiated, but not undifferentiated, human neural progenitor NT2 cells and to regulate a transcriptional repressor in such cells (Kawasaki and Taira, 2003a). Other researchers have identified the generation of intron-derived micro-RNA-like molecules (Id-micro-RNA) from these regions as a tool for analysis of gene function and development of gene-specific therapeutics, and predicted possible applications including major gene modulation systems for developmental regulation, intracellular immunity, heterochromatin inactivation, and genomic evolution in eukaryotes (Lin and Ying, 2004b). However, no reports referred to regulating the cellular and organismal capacities to confront stressful insults.

Micro-RNAs have been implicated in various neurological diseases such as Fragile X syndrome, spinal muscular atrophy (SMA), early onset parkinsonism (Waisman syndrome) and X-linked mental retaradation (MRX3), as well as various cancers and precancerous conditions such as Wilm's tumor, testicular germ cell tumor, chronic lymphocytic leukemia (CLL), B cell leukemia, precancerous and neoplastic colorectal tissues and Burkkit's lymphoma [reviewed in Gong H, et al., 2004, Medical Research Reviews, Published online in Wiley InterScience (www.interscience.wiley.com)].

Recent in vitro studies utilizing 2′-O-methyl oligoribonucleotides directed against the miR-21 micro-RNA resulted in reversal of EGFP expression in HeLa cells transformed to express exogenous EGFP siRNA (Meister G, et al., 2004, RNA 10: 544-550). In addition, 2′-O-methylated oligos directed against the let-7 micro-RNA of C. elegans were shown to suppress the effect of an exogenous let-7 micro-RNA assembled to the RISC complex (Hutvagner G, et al., 2004, PLoS BIOLOGY 2: 465-475). Moreover, specific inhibition of miR-143 micro-RNA using an antisense oligonucleotide resulted in inhibition of adipocyte differentiation (Esau C, et al., 2004, J. Biol. Chem. 279: 52361-5).

However, the involvement and function of micro-RNA components in AChE-related biological pathways have not been studied yet.

### SUMMARY OF THE INVENTION

While reducing the present invention to practice, the present inventor has uncovered that ACHE associated biological pathways can be regulated by controlling the level of AChE-related micro-RNA.

Hence, it is an object of the present invention to provide novel agents for controlling the level of AChE-related micro-RNA in cells and organisms.

It is another object of the present invention to provide pharmaceutical compositions containing these agents.

It is another object of the present invention to provide therapeutic methods using the agents and/or pharmaceutical compositions.

According to one aspect of the present invention there is provided a method of regulating an AChE-associated biological pathway having a miRNA component, the method comprising subjecting the AChE-associated biological pathway to an agent capable of regulating a function of the miRNA, thereby regulating the ACHE-associated biological pathway.

According to another aspect of the present invention there is provided a method of regulating an expression level ratio of ACHE-S and ACHE-R and/or ACHE-S mRNA and AChE-R mRNA splice variants in AChE expressing cells comprising subjecting the AChE gene expressing cells to an agent capable of regulating a function of a miRNA component associated with regulating the expression level ratio of ACHE-S and AChE-R splice variants, thereby regulating the expression level and biochemical properties associated with the ACHE-S and AChE-R splice variants in the AChE expressing cells.

According to yet another aspect of the present invention there is provided a method of treating a pathology related to an AChE-associated biological pathway, the method comprising administering to a subject in need thereof an agent capable of regulating a function of a miRNA component of the AChE-associated biological pathway, thereby treating the pathology.

According to still another aspect of the present invention there is provided a method of altering differentiation and/or proliferation of hematopoietic progenitor and/or stem cells, the method comprising subjecting the progenitor and/or stem cells to an agent capable of regulating a function of a miRNA component of an ACHE-associated biological pathway in the progenitor and/or stem cells, thereby altering differentiation and/or proliferation of the hematopoietic progenitor and/or stem cells.

According to an additional aspect of the present invention there is provided a method of regulating apoptosis in cells and/or a tissue of a subject in need thereof, the method comprising subjecting the cells and/or the tissue of the subject to an agent capable of regulating a function a miRNA component of an AChE-associated biological pathway in the cells and/or tissue, thereby regulating apoptosis in the cells and/or the tissue of the subject.

According to yet an additional aspect of the present invention there is provided a method of treating a pathology related to an AChE-associated biological pathway comprising administering to a subject in need thereof a polynucleotide selected from the group consisting of a polynucleotide which comprises at least 10 consecutive nucleotides of the nucleic acid sequence set forth in SEQ ID NO:1, a polynucleotide hybridizable in cells under physiological conditions to an RNA molecule which comprises a nucleic acid sequence as set forth in SEQ ID NO:2, a polynucleotide as set forth by SEQ ID NO:1, a polynucleotide which comprises at least 10 consecutive nucleotides of the nucleic acid sequence set forth in SEQ ID NO:2, a polynucleotide hybridizable in cells under physiological conditions to an RNA molecule which comprises a nucleic acid sequence as set forth in SEQ ID NO:21 and/or 22, a polynucleotide as set forth by SEQ ID NO:2, a polynucleotide which comprises at least 25 consecutive nucleotides of the nucleic acid sequence set forth in SEQ ID NO:13, a polynucleotide as set forth by SEQ ID NO:13, a polynucleotide which comprises at least 20 consecutive nucleotides of SEQ ID NO:13 and/or at least 10 consecutive nucleotides of SEQ ID NO:1, a polynucleotide as set forth by SEQ ID NO:12 or a functional homolog thereof, a polynucleotide as set forth by SEQ ID:19 or a functional homolog thereof, thereby treating the pathology related to an AChE-associated biological pathway.

According to still an additional aspect of the present invention there is provided a method of treating a disease or condition in which regulating nitric oxide levels is therapeutically beneficial in a subject, the method comprising administering to a subject in need thereof an agent capable of regulating a miRNA component of an AChE-associated biological pathway, thereby treating the disease or condition in which regulating nitric oxide levels is therapeutically beneficial.

According to still an additional aspect of the present invention there is provided a method of diagnosing a pathology associated with abnormal function of a miRNA component of an AChE-associated biological pathway in a subject, the method comprising obtaining a biological sample from the subject and determining a level of the miRNA in cells of said biological sample, wherein a level of the miRNA above or below a predetermined threshold or range is indicative of a presence of a pathology associated with abnormal function of the miRNA.

According to a further aspect of the present invention there is provided an isolated polynucleotide comprising a nucleic acid sequence which comprises at least 10 consecutive nucleotides of the nucleotide sequence set forth in SEQ ID NO:1.

According to yet a further aspect of the present invention there is provided an isolated polynucleotide comprising a nucleic acid sequence of 10-50 bases and capable of hybridizing in cells under physiological conditions with an RNA molecule which comprises a nucleotide sequence as set forth in SEQ ID NO:2.

According to still a further aspect of the present invention there is provided a pharmaceutical composition comprising as an active ingredient the polynucleotide which comprises a nucleic acid sequence of 10-50 bases and capable of hybridizing in cells under physiological conditions with an RNA molecule which comprises a nucleotide sequence as set forth in SEQ ID NO:2 and a pharmaceutically acceptable carrier.

According to still a further aspect of the present invention there is provided an isolated polynucleotide comprising a nucleic acid sequence which comprises at least 10 consecutive nucleotides from the nucleotide sequence set forth by SEQ ID NO:2.

According to still a further aspect of the present invention there is provided an isolated polynucleotide comprising a nucleic acid sequence of 10-50 bases and capable of hybridizing in cells under physiological conditions with an RNA molecule which comprises a nucleotide sequence as set forth in SEQ ID NO:1.

According to still a further aspect of the present invention there is provided a pharmaceutical composition comprising, as an active ingredient the polynucleotide which comprises a nucleic acid sequence of 10-50 bases and capable of hybridizing in cells under physiological conditions with an RNA molecule which comprises a nucleotide sequence as set forth in SEQ ID NO:1 and a pharmaceutically acceptable carrier.

According to still a further aspect of the present invention there is provided an isolated polynucleotide comprising a nucleic acid sequence which comprises at least 20 consecutive nucleotides from the nucleotide sequence set forth in SEQ ID NO:13.

According to still a further aspect of the present invention there is provided a pharmaceutical composition comprising, as an active ingredient, the polynucleotide which comprises a nucleic acid sequence which comprises at least 20 consecutive nucleotides from the nucleotide sequence set forth in SEQ ID NO:13 and a pharmaceutically acceptable carrier.

According to further features in preferred embodiments of the invention described below, the agent is a polynucleotide.

According to still further features in the described preferred embodiments the polynucleotide is a modified polynucleotide.

According to still further features in the described preferred embodiments the modified polynucleotide is at least partially 2′-oxymethylated.

According to still further features in the described preferred embodiments the modified polynucleotide is a fully 2′-oxymethylated polynucleotide.

According to still further features in the described preferred embodiments the fully 2′-oxymethylated polynucleotide is set forth by SEQ ID NO:23 or 24.

According to still further features in the described preferred embodiments the miRNA is set forth by SEQ ID NO:21 and/or 22.

According to still further features in the described preferred embodiments the miRNA is set forth by a sequence selected from the group consisting of SEQ ID NOs: 54, 93, 94, 98, 99, 100, 107 and 108.

According to still further features in the described preferred embodiments regulating said function of said miRNA is upregulating.

According to still further features in the described preferred embodiments regulating said function of said miRNA is downregulating.

According to still further features in the described preferred embodiments the regulating said function of said miRNA is upregulating and whereas a sequence of said polynucleotide comprises at least 10 consecutive nucleotides from the nucleic acid sequence set forth by SEQ ID NO:1.

According to still further features in the described preferred embodiments regulating said function of said miRNA is upregulating and whereas a sequence of said polynucleotide is hybridizable in cells under physiological conditions to an RNA molecule which comprises a nucleic acid sequence as set forth by SEQ ID NO:2.

According to still further features in the described preferred embodiments regulating said function of said miRNA is upregulating and whereas a sequence of said polynucleotide is as set forth in SEQ ID NO:1.

According to still further features in the described preferred embodiments regulating said function of said miRNA is downregulating and whereas a sequence of said polynucleotide comprises at least 10 consecutive nucleotides of the nucleic acid sequence set forth in SEQ ID NO:2.

According to still further features in the described preferred embodiments regulating said function of said miRNA is downregulating and whereas a sequence of said polynucleotide is hybridizable in cells under physiological conditions to an RNA molecule which comprises a nucleic acid sequence as set forth by SEQ ID NO:21 and/or 22.

According to still further features in the described preferred embodiments regulating said function of said miRNA is downregulating and whereas a sequence of said polynucleotide is as set forth in SEQ ID NO:2.

According to still further features in the described preferred embodiments regulating said function of said miRNA is upregulating and whereas a sequence of said polynucleotide comprises at least 25 consecutive nucleotides of the nucleic acid sequence set forth in SEQ ID NO:13.

According to still further features in the described preferred embodiments regulating said function of said miRNA is upregulating and whereas a sequence of said polynucleotide is as set forth in SEQ ID NO:13.

According to still further features in the described preferred embodiments regulating said function of said miRNA is upregulating and whereas said polynucleotide comprises at least 20 consecutive nucleotides of SEQ ID NO:13 and/or at least 10 consecutive nucleotides of SEQ ID NO:1.

According to still further features in the described preferred embodiments the AChE-associated biological pathway regulates hematopoiesis and/or an immune reaction.

According to still further features in the described preferred embodiments regulating said function of said miRNA is upregulating and whereas said polynucleotide is set forth by SEQ ID NO:12 or a functional homolog thereof.

According to still further features in the described preferred embodiments regulating said function of said miRNA is downregulating and whereas said polynucleotide is set forth by SEQ ID: 19 or a functional homolog thereof.

According to still further features in the described preferred embodiments the AChE-associated biological pathway regulates megakaryocyte proliferation and/or differentiation.

According to still further features in the described preferred embodiments the AChE-associated biological pathway regulates apoptosis.

According to still further features in the described preferred embodiments the AChE-associated biological pathway regulates nitric oxide levels.

According to still further features in the described preferred embodiments the pathology is characterized by an aberrant cholinergic signaling.

According to still further features in the described preferred embodiments the pathology is characterized by an abnormal hematopoeitic cell proliferation and/or differentiation.

According to still further features in the described preferred embodiments the pathology is characterized by an abnormal megakaryocyte proliferation and/or differentiation.

According to still further features in the described preferred embodiments the pathology is selected from the group consisting of thrombocytopenia, idiopathic thrombocytopenic purpura (ITP), congenital amegakaryocytic thrombocytopenia (CAMT), essential thrombocythemia (ET) and acquired amegakaryocytic thrombocytopenia (AATP).

According to still further features in the described preferred embodiments the pathology is characterized by cellular stress.

According to still further features in the described preferred embodiments the pathology is caused by drug poisoning.

According to still further features in the described preferred embodiments the pathology is caused by exposure to inflammatory response-inducing agents.

According to still further features in the described preferred embodiments the pathology is caused by exposure to organophosphates.

According to still further features in the described preferred embodiments the pathology is characterized by abnormal apoptosis.

According to still further features of the described preferred embodiments the pathology is caused by exposure to ACHE inhibitors.

According to still further features in the described preferred embodiments the abnormal apoptosis is characterized by reduced level of apoptosis and whereas said pathology is selected from the group consisting of psoriasis, ichythyiosis, common warts, keratoacanthoma, seborrhoic keratosis, seborrhea, squamous cell carcinomas (SCC), basal cell carcinoma (BCC), non-melanoma skin cancer (NMSC) and multiple human tumors.

According to still further features in the described preferred embodiments the abnormal apoptosis is characterized by increased level of apoptosis and whereas said pathology is selected from the group consisting of an autoimmune disease and a vascular disease.

According to still further features in the described preferred embodiments the pathology is selected such that regulating NO levels is therapeutically beneficial.

According to still further features in the described preferred embodiments the pathology is selected from the group consisting of angina pectoris, ischemic disease, congestive heart failure, hypertension, pulmonary hypertension, stroke, inflammation, a bacterial infection, a viral infection, a parasitic infection, an immune disease, a tumor, impotence, hypothermia, abnormal wound healing, a leg ulcer, alopecia, decreased long-term potenetiation, a neurodegenerative disorder and diabetes.

According to still further features in the described preferred embodiments the disease or condition is selected from the group consisting of angina pectoris, ischemic disease, congestive heart failure, hypertension, pulmonary hypertension, stroke, inflammation, a bacterial infection, a viral infection, a parasitic infection, a tumor, impotence, hypothermia, abnormal wound healing, a leg ulcer, alopecia, decreased long-term potenetiation, a neurodegenerative disease and diabetes.

According to still further features in the described preferred embodiments the hematopoietic progenitor cells are megakaryoblasts.

According to still further features in the described preferred embodiments the miRNA component is set forth by SEQ NO:21.

According to still further features in the described preferred embodiments the miRNA component is set forth by SEQ ID NO:22.

According to still further features in the described preferred embodiments the determining is effected using an oligonucleotide.

According to still further features in the described preferred embodiments the oligonucleotide is specifically hybridizable with said miRNA under stringent hybridization conditions.

According to still further features in the described preferred embodiments the oligonucleotide is capable of specifically hybridizing with a polynucleotide having a nucleic acid sequence as set forth by SEQ ID NO:21 and/or 22 under stringent hybridization conditions.

According to still further features in the described preferred embodiments the determining is effected using at least one oligonucleotide capable of specifically amplifying a polynucleotide having a nucleic acid sequence as set forth in SEQ ID NO:21 and/or 22.

According to still further features in the described preferred embodiments the biological sample is selected from the group consisting of blood, bone marrow, intestine, spinal fluid and cord blood.

According to still further features in the described preferred embodiments the determining is effected using a method selected from the group consisting of an RNA-based hybridization method and reverse transcription-based detection method.

According to still further features in the described preferred embodiments the RNA-based hybridization method is selected from the group consisting of Northern blot hybridization, RNA in situ hybridization and chip hybridization, e.g., spotted or lithography-prepared chip.

According to still further features in the described preferred embodiments the reverse transcription-based detection method is selected from the group consisting of RT-PCR, quantitative RT-PCR, real-time reverse transcription PCR, semi-quantitative RT-PCR, in situ RT-PCR, primer extension, mass spectroscopy, sequencing, sequencing by hybridization, LCR (LAR), Self-Sustained Synthetic Reaction (3SR/NASBA), Q-Beta (Qb) Replicase reaction, cycling probe reaction (CPR), a branched DNA analysis, and detection of at least one nucleic acid change.

According to still further features in the described preferred embodiments the detection of at least one nucleic change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

According to still further features in the described preferred embodiments the predetermined threshold and/or range is calculated from biological samples obtained from at least two individuals who do not suffer from the pathology.

According to still further features in the described preferred embodiments the isolated polynucleotide is with the proviso that the isolated polynucleotide is not identical to the sequence set forth in SEQ ID NO:1.

According to still further features in the described preferred embodiments the isolated polynucleotide of claim 149, with the proviso that the isolated polynucleotide is not identical to the sequence set forth in SEQ ID NO:1.

According to still further features in the described preferred embodiments the isolated polynucleotide with the proviso that the isolated polynucleotide is not identical to the sequence set forth in SEQ ID NO:13.

According to another aspect of the present invention there is provided a method of treating a pathology associated with abnormal levels of AChE-S or AChE-R splice variants, the method comprising administering to a subject in need thereof an agent capable of regulating a function of a micro-RNA component of an AChE-associated biological pathway, thereby treating the pathology. Increased levels of ACHE-S are characteristic of astrocyte tumor cells, Alzheimer's disease (AD) and Parkinsonism. Abnormaly increased levels of the ACHE-R are further characteristic of Myasthenia gravis (MG), lung cancer, such as small cell lung carcinoma, stress disorder, such as, for example, acute stress, transient post traumatic stress disorder and persistent post traumatic stress disorder, panic disorder, glioblastoma, enhanced fear memory and/or long-term potentiation, male infertility, exposure to bacterial infection and behavioral impairment. All these diseases can hence be treated using the therapeutic agents described herein.

The present invention successfully addresses the shortcomings of the presently known configurations by providing agents and polynucleotides capable of regulating the function of an AChE-related micro-RNA, such as AChmiRNA.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show details of the invention in more detail than is necessary for a fundamental understanding of the invention, the description taken with the drawings making apparent to those skilled in the art how the several forms of the invention may be embodied in practice.

In the drawings:

FIGS. 1a-b are schematic illustrations depicting the proposed mechanism of RNA interference (adopted from Hannon, 2002). FIG. 1a depicts the enzyme Dicer (a dimer here shown simplified, with only two domains per subunit) processing long dsRNA into 21- to 23-bp siRNAs, which are then incorporated into the RNA-induced silencing complex (RISC). RISC then cleaves target mRNA in a sequence-dependent manner, silencing gene expression. FIG. 1b depicts the proposed mechanism by which Dicer cleaves dsRNA into siRNA products.

FIGS. 2a-c depict the effect of Thapsigargin on miRNA-181a precursor RNA levels. FIG. 2a—illustrates the sequence of miRNA-181a precursor RNA (natural—SEQ ID NO:22; synthetic—SEQ ID NO:13; miRNA Registry website <http://www.sanger.ac.uk/Software/Rfam/mirna/index.shtml>). FIG. 2b—illustrates the stem-loop structure of human (h) pre-miRNA181 (SEQ ID NO:13) and its folding energy as predicted by the MFOLD algorithm (http://bioweb.pasteur.fr/seqanal/interfaces/mfold-simple). FIG. 2c is a bar graph depicting quantification of LightCycler® PCR using the hmiRNA-181a primers [SEQ ID NO:6 (5′-GGTACAGTCAACGGTCAGTGG-3′) and SEQ ID NO:7 (5′-GGACTCCAAGGAACATTCAACG-3′);] in cultured human Meg-01 cells following the indicated treatments. Note that Thapsigargin caused a significant decrease in the levels of the miRNA-181a (AChmiRNA) amplicon (SEQ ID NO:14; 5′-GGACTCCAAGGAACATTCAACGCTGTCGGTGAGTTTGGGATTTGAAAAAA CCACTGACCGTTGACTGTACC-3′) in a manner dependent on the enzyme activities of PKA, PKC and AChE. Shown are averages from 3 or more representative measurements (values=average±S.E.M.).

FIGS. 3a-f are scanning electron microscopy of untreated (Control, CT; FIGS. 3a and d), Thapsigargin-treated (FIGS. 3b and c) and ARP (SEQ ID NO:3; FIGS. 3e and f)—treated Meg-01 cells. Note that while control cells exhibit a smooth surface (FIGS. 3a and d), cells treated for 24 hours with either Thapsigargin (FIG. 3c) or ARP (FIG. 3e) show initial formation of flat membrane sheets or elongated pseudopodia reflecting proplatelet formation territories (FIGS. 3c and f), which are characteristic of megakaryocytic differentiation.

FIGS. 4a-e depict the polidy of Meg-01 cells using fluorescent-activated cell sorter (FACS) raw data (FIGS. 4a-d) and quantification FACS analysis (FIG. 4e). FIG. 4a—control (CTR), untreated cells; FIG. 4b-Thapsigargin (Thapsi) treated cells; FIG. 4c—ARP (SEQ ID NO;3) treated cells; FIG. 4d—PMA treated cells. Note that both ARP and Thapsi increased the ploidy of Meg-01 cells following 72 hours but not 24 or 48 hours (data not shown). PMA was used as a positive control. FIG. 4e—is a bar graph illustrating the quantification of cell populations identified by FACS. Results are presented as percentage of cells (average±s.e.m) in each category (i.e., ploidy). *=p<0.01 vs. control; **=p<0.05 vs. control.

FIGS. 5a-b are bar graphs depicting nuclear area measurement of Meg-01 cells treated for 24 hours with either ARP (SEQ ID NO:3; FIG. 5a) or Thapsigargin (Thapsi; FIG. 5b). Note that although increase in DNA content was not yet detected by FACS at 24 hours, the nuclear area was already increased by this time, suggesting that ER-calcium release (Thapsigargin) and the induction of overproduced AChE-R (by its cleavable C-terminal peptide ARP) lead to Meg-01 cells to differentiation.

FIG. 6 is a scatter diagram depicting quantification of GATA-1 immunocytochemistry (arbitrary units of labeling density). GATA-1 is a transcription factor known to participate in the differentiation of megakaryocytes. Increased intensity of staining for GATA-1 correlated with the increase in nuclear area in Meg-01 cells treated either with ARP (SEQ ID NO:3) or Thapsigargin (Thapsi), but not in the control (CT) cells. Control: R2=0.0006; Thapsi: R2=0.1992; ARP: R2=0.1304.

FIG. 7 is a schematic illustration depicting the experimental paradigm based on the following assumptions: Thapsigargin releases intracellular Ca++ stores from the ER into the cytoplasmic space; This blocks TFIIIB/C, the transcription factor responsible for the synthesis of RNA polymerase III; RNApolIII initiates the production of all micro-RNAs. Therefore, blocking TFIIIB/C will rapidly cause a reduction in AChmiRNA. Such signals which induce intracellular Ca++ release and AChmiRNA reduction also induce the accumulation of ACHE-R mRNA, suggesting that AChE-R mRNA serves as a direct or indirect target for AChmiRNA-induced destruction; When ACHE-R mRNA accumulates, its AChE-R protein product is cleaved at the C-terminus (Cohen et al., J. Mol. Neurosc. 2003) to yield the ARP peptide with its independent growth factor capacities; In ARP-treated cells, Caspase-3 and AChE mRNA variants are overproduced, demonstrating an auto-regulated property of ARP. This indicates causal involvement of AChmiRNA reduction in caspase-3 accumulation; Intracellular Ca++ release also induces c-myc, which in turn induces ACHE gene expression through an additional pathway.

FIGS. 8a-b depict the increase in AChE-R mRNA following ARP (SEQ ID NO:3) or Thapsigargin (Thapsi) treatment. FIG. 8a—a bar graph depicting the fold increase in the intensity of the AChE-R RT-PCR signal in ARP or Thapsigargin-treated cells as compared with controls. Values present average±S.E.M. FIG. 8b—raw data of RT-PCR analysis. Lane 1—MW marker, lane 2—control cells, lane 3—ARP-treated cells, lane 4—Thapsigargin-treated cells.

FIG. 9 is a bar graph depicting the population distribution of a quantification of fluorescent in situ hybridization staining of ACHE-R mRNA in thapsigargin- or ARP-treated Meg01 cells. CT—control; Thapsi—Thapsigargin; ARP—SEQ ID NO:3; au=arbitrary units of fluorescence signal. Note that while in control cells ACHE-R mRNA displayed a normal Gaussian distribution, in both ARP and Thapsi-treated cells, the fraction of cells with higher fluorescence levels is increased.

FIG. 10 is a bar graph depicting the relative AChmiRNA concentration following treatment with ARP (SEQ ID NO:3), BIM (a PKC inhibitor) or H89 (a PKA inhibitor). RT-PCR was performed in cultured human Meg-01 cells following the indicated treatments using the LightCycler® PCR and AChmiRNA primers (SEQ ID NOS: 6 and 7). Note the significant decrease in AChmiRNA levels (amplicon; SEQ ID NO:14) in ARP-treated cells, and abolishment of this decrease by BIM or H89 demonstrating the links to both cholinergic signaling and signal transduction by PKC and PKA. Shown are averages from 3 or more representative measurements (values=average±S.E.M.).

FIGS. 11a-b are photomicrographs depicting immunostaining with an anti-activated caspase-3 antibody in control (CTR, FIG. 11a) or Thapsigargin (Thapsi; FIG. 11b) Meg-01 cells. Arrows show positive cells.

FIGS. 12a-c depict changes in immunoreactivity of caspase-3, as compared with in situ hybridization signals for AChE-S and ACHE-R mRNA in Meg-01 cells following Thapsigargin (FIGS. 12a and c) or ARP (SEQ ID NO:3; FIG. 12b) treatment. Meg-01 cells were treated for 24 hours with either Thapsigargin or ARP26 (SEQ ID NO:3) and immunostaining, or in situ hybridization, was performed using antibodies or cDNA probes specific to the noted proteins or transcripts. FIG. 12a—is a bar graph illustrating the percent of positive cells (out of total cells) prior (−) or following (+) Thapsigargin treatment. Note the increase in the labeling for AChE-R mRNA and caspase-3 as compared with the decrease in expression of ACHE-S mRNA. FIG. 12b—is a graph depicting the fold increase of positive cells following 24 hours of incubation with increasing concentrations of ARP. Note that ARP26 induced an increase in the expression of ACHE-R mRNA and a decrease in the expression of ACHE-S mRNA. ARP also increased the fraction of cells immunopositive for activated caspase-3. FIG. 12c—is a bar graph depicting caspase-3 fold increase in Thapsigargin-treated Meg-01 cells, in the presence or absence (−) of Actinomycin D (ActD; an inhibitor of transcription). Note that Actinomycin D blocked the effect of Thapsi on caspase-3 activation. Values present average±S.E.M.

FIG. 13 is a schematic illustration depicting that the intrinsic apoptosis pathway leads to caspase-3 activation through the mitochondrial pathway.

FIGS. 14a-g depict that Meg-01 differentiation involves a caspase-activation cascade. FIGS. 14a-c are images obtained from transmission electron microscopy of control (FIG. 14a), Thapsigargin-treated (FIG. 14b) or ARP (SEQ ID NO:3)—treated (FIG. 14c) Meg-01 cells. Note that cells treated with either ARP or Thapsigargin show no chromatin condensation. Rather, membrane blebbing and maintenance of organelle integrity (regarded as apoptotic features, but are also related to megakaryocytic maturation) are observed. Cytoplasmic vacuolization, besides membrane blebbing, is compatible with the platelet-forming process. [symbol] : mitochondria; n: nucleus; arrow: membrane blebbing. FIG. 14d—a graph depicting the quantification of immunostaining of activated caspase-3 in Meg-01 cells treated with either Thapsi or ARP for 24 hours in the presence of Bongkrekic acid, an inhibitor of the mitochondrial permeability transition pore, which blocked both Thapsi and ARP effects on caspase-3 activation. FIG. 14e—a graph depicting activated caspase-9 immunostaining quantification. FIG. 14f—a bar graph depicting quantification of Bcl-2 immunostaining. FIG. 14g—a bar graph depicting quantification of TUNEL staining. All graphs data (FIGS. 14d-g) present average±S.E.M.

FIGS. 15a-d depict the sequence (FIG. 15a) and effects of AChmiON on apoptosis (FIG. 15b), BrDU incorporation (FIG. 15c) and cell adhesion (FIG. 15d). FIG. 15a—depicts the sequence of the AChmiON synthetic oligonucleotide (SEQ ID NO:1) which mimics miRNA-181a micro-RNA. Full 2′-O-methyl protection served to prevent nucleolytic degradation (SEQ ID NO:23). FIG. 15b is a bar graph depicting the quantification of a TUNEL assay in controls, Thapsigargin (Thapsi)-treated or AChmiON-treated Meg-01 cells. Note the increase in TUNEL staining in cells treated with the AChmiON (SEQ ID NO:23). FIG. 15c is a bar graph depicting the quantification of a BrdU incorporation into Meg-01 cells treated with Thapsi or AChmiON. BrdU incorporation was measured 72 hours following Thapsi and/or AChmiON treatment. FIG. 15d is a bar graph depicting an adhesion assay performed 72 hours following Thapsi treatment and/or AChmiON treatment. Values present average±S.E.M.

FIGS. 16a-d are photomicrographs depicting fluorescent in situ hybridization for AChE-R mRNA (FIGS. 16a and b) and AChE-S mRNA (FIGS. 16c and d). Note that in control cells (FIG. 16a), AChE-S mRNA signals were higher than in thapsi-treated cells (FIG. 16b). On the other hand, Thapsi treatment increased the level of AChE-R mRNA (FIG. 16d), which is low in control cells (FIG. 16c).

FIG. 17 depicts Northern Blot analysis of miRNA181a in Meg-01 cells following treatment with Thapsi, AChmiON and/or Anti-miR181. Lane 1—control, untreated cells; lane 2—cells treated with Thapsi; lane 3—cells treated with AChmiON (SEQ ID NO:23); lane 4—cells treated with anti-miR181 (SEQ ID NO:24); lane 5—cells treated with both AChmiON and anti-miR181; lane 6—cells treated with both Thapsi and AChmiON; lane 7—cells treated with both Thapsi and anti-miR181; lane 8—cells treated with both Thapsi, AchmiON and anti-miR181. Note the effect of anti-miR181 in reducing the level of miRNA 181a in the presence or absence of Thapsi and/or AChmiON.

FIGS. 18a-c depict c-Myc immunohistochemistry. FIGS. 18a and b are photomicrographs depicting C-Myc immunohistochemistry in controls (FIG. 18a) and Thapsi-treated (FIG. 18b) Meg-01 cells. FIG. 18c is a bar graph depicting the quantification of c-Myc immunohistochemistry. Note that the increase in c-Myc induced by Thapsi is not affected by AChmiON (values=average±s.e.m). This suggests that c-myc is not a target of miRNA-181a (AChmiRNA) yet shows that the increase in ACHE-R mRNA under thapsigargin is largely due to a shifted splicing, and/or increased stability of AChE-RmRNA, not transcriptional activation by c-myc.

FIGS. 19a-e depict that ARP and Thapsi effects depend on PKA and PKC and that Thapsi effects further depend on AChE. FIG. 19a is a schematic illustration depicting the interaction of AChE-R with PKCβII through RACK1. FIG. 19b is a bar graph depicting the quantification of activated caspase-3 immunohistochemistry on Meg-01 cells treated with the noted drugs and presented as fold increase in treated cells as compared with control cells. Meg-01 cells were treated for 24 hours with ARP or Thapsi in the presence of the PKC inhibitor bisindolylmaleimide (BIM), or H89, an inhibitor of PKA. Note that BIM and H89 inhibited the activation of caspase-3 induced either by ARP or Thapsi. FIG. 19c is a graph depicting quantification of PKCβII immunocytochemistry presented as fold increase in treated cells as compared with control cells. Meg-01 cells were induced for 24 hours with ARP, Thapsi or PMA (positive control for megakaryocytic differentiation). All treatments increased staining intensity for PKCβ. FIG. 19d is a bar graph depicting the quantification of AChE-R immunocytochemistry presented as fold increase in treated cells as compared with control cells. Meg-01 cells were treated for 24 hours with Thapsi in the presence of BIM or H89. Note that both BIM and H89 prevented the increase in AChE-R induced by Thapsi. FIG. 19e is a bar graph depicting the effect of AChE inhibitors upon caspase-3 activation. EN101 (SEQ ID NO:5), an antisense oligonucleotide suppressing AChE-R mRNA, blocked caspase-3 activation, confirming the participation of AChE-R in the signaling pathway induced by Thapsi. Physostigmine and Pyridostigmine, small molecule inhibitors of AChE, inhibited the activation of caspase-3 induced by Thapsi. Values present average±S.E.M. in all graphs (FIGS. 19b-e).

FIGS. 20a-b depict the in vivo levels of AChmiRNA under neurological and immunological stressors. The in vivo levels of AChmiRNA were determined from total RNA using a quantitative RT-PCR in bone marrow of LPS challenged mice. FIG. 20a—a bar graph depicting the quantification of an RT-PCR analysis of AChmiRNA. Control (FVB/N) and AChE-R transgenic (TgR) female mice were intraperitoneally (I.P.) injected with the salmonella lipopolysacharide (LPS) at a dose of 50 μg LPS in 200 μl PBS per mouse. Mice were sacrificed at the indicated time points (0, 24 or 48 hours) following treatment and total RNA was extracted from the indicated tissues. Note the decrease in AChmiRNA at 24 and 48 hours following LPS administration in control mice and the even more pronounced decrease in TgR mice. FIG. 20b—a bar graph depicting a quantification of an RT-PCR analysis in the intestine of PO and MPTP challenged mice. Male mice, transgenic for ACHE-R (TgR), were IP injected with Paraoxon at 2 injections, each of 0.5 mg/kg, at a 4 hour interval. MPTP was also given I.P. in 4 injections of 20 mg/kg each, at 2 hours intervals. Thus, the two Paraoxon injections coincided with the first and third MPTP injections. Mice were sacrificed 7 days after treatment. Note the significant decrease of AChmiRNA level in mice treated with either PO or MPTP and the even more pronounced decrease in mice treated with both agents (i.e., a synergistic effect). Error bars±St. Dev. from triplicates.

FIG. 21 is a bar graph depicting the effect of CpG ODN2216 (SEQ ID NO:12) on AChmiRNA levels in human PBMC. Total RNA was isolated from pooled peripheral blood mononuclear cells (PBMC) using Trizol. AChmiRNA expression was assayed by quantitative RT-PCR. Note the significant increase in AChmiRNA level following administration of the CpG ODN2216. Thus, the effect of the CpG ODN2216 is inverse to that of Thapsigargin, LPS, paraoxon or MPTP. Error bars—St. Dev. from 5 measurements.

FIG. 22 is a bar graph depicting AChmiRNA, TFIIIA, TFIIIB and the splicing factor ASF/SF₂ expression in PBMC cells treated with ODN 2006 or ODN 2216 oligonucleotides, known to exert their effects through distinct TLR members. Real-time RT-PCR was performed simultaneously for the noted transcripts.

FIG. 23 is a bar graph depicting the quantification of a TUNEL assay in controls, Thapsigargin (Thapsi)-treated, AChmiON-treated, or antisense AChmiON-treated Meg-01 cells. Note the increase in TUNEL staining in cells treated with the AChmiON (SEQ ID NO:23) and the normal level of TUNEL staining in cells treated with the antisense to AChmiON (SEQ ID NO:24).

FIGS. 24a-j depict the population distribution of quantification of fluorescent in situ hybridization staining for ACHE-S mRNA (FIGS. 24a, c, e, g, i) and AChE-R mRNA (FIGS. 24b, d, f, h and j). Thapsi decreased the fractions of cells with high levels of ACHE-S mRNA (FIG. 24a) while increasing those fractions with high ACHE-R mRNA (FIG. 24b). On the other hand, AChmiON (SEQ ID NO:23) increased the fractions of cells with high ACHE-S mRNA (FIG. 24c) but did not reduce AChE-R mRNA levels (FIG. 24d). In contrast, treatment of cells with the antisense AChmiON (SEQ ID NO:24) resulted in marginal effect on both ACHE-S and AChE-R (FIGS. 24e and f, respectively). On the other hand, while co-treatment of cells with both Thapsi and AChmiON (SEQ ID NO:23) results in a significant increase AChE-S (FIG. 24g) and a decrease in AChE-R (FIG. 24h), co-treatment of cells with both Thapsi and antisense AChmiON (SEQ ID NO:24) prevents the increase in ACHE-S (FIG. 24i) and induces a further increase in ACHE-R (FIG. 24j). Ct=control; Thapsi (T)—Thapsigargin; miRNA 181=AChmiON (SEQ ID NO:23); antimiRNA 181=antisense to AChmiON (SEQ ID NO:24); au=arbitrary units.

FIG. 25 is a scheme depicting the working hypothesis of the present invention. Both the Ca²⁺ releasing agent Thapsigargin and the AChE-R C-terminal cleavable peptide ARP (SEQ ID NO:3) initiate a cascade reaction with differentiation and stress hallmarks in the promegakaryocytic cell line Meg-01. However, the mechanisms involved are likely distinct. Thus, Thapsigargin blocks TFIII functioning, reducing RNA Polymerase III levels and consequently suppressing AChmiRNA, which prevents destruction of AChE-R mRNA. On the other hand, ARP induces RNA Polymerase II, enhancing AChE-R mRNA production. Both agents also induce c-myc in a PKC and PKA-inhibitable manner and lead to differentiation hallmarks including elevated BrdU incorporation, reflecting nuclear endoreduplication, Caspase-3 activation and intensified cell adhesion. In contrast to these parallel effects, either cholinergic signals or the synthetic AChmiRNA mimic AChmiON block BrdU incorporation, caspase-3 activation and elevated adhesion while inducing Tunel reaction reflecting apoptotic events but not inducing the shift from AChE-S to AChE-R which occurs under Thapsigargin.

FIG. 26 is a scheme depicting that downregulation of the stress-induced soluble form of AChE by CpG-induced AChmiRNA can enhance cholinergic signals. The TLR9 ligand of CpG ODN amplifies the expression of AChEmiRNA, ensuring suppressed levels of soluble ACHE-R. As a consequence, diminished degradation of ACh by the soluble esterase can increase the levels of cholinergic signals (ACh), in cholinergic and non-cholinergic neurons, muscle, gland or blood cells, all of which carry ACh receptors (AChR). Increased cholinergic signals impact both on immune cell subsets and the nervous system. Thereby, the recognition of CpG by the immune system increases the activity of cholinergic nerves, and increased activity of cholinergic nerves affects the activity of immune cell subsets. Thus the cholinergic system forms an interface between the nervous system and immunity through CpG-mediated miRNA signals.

FIGS. 27a-c are bar graphs depicting the quantification of nitric oxide in raw 264.7 macrophages incubated in the presence of Hen-101, inv Hen-101, AChmion, LPS, Hen-101 (antisense suppressing AChE-R mRNA levels) and interferon-γ, inv Hen-101 and interferon-γ, AChmion and interferon-γ, LPS and interferon-γ, interferon-γ and control (change of medium only) following 6 (FIG. 27a), 12 (FIG. 27b) and 24 (FIG. 27c) hours. Note the delayed increase in NO in cells treated with the AChmiON (SEQ ID NO:23) compared with cells treated with interferon-γ and LPS.

FIG. 28 is a graph comparing LR values from LPS challenged to naïve cells, and LPS+EN101-challenged cells to cells treated with EN101 alone.

FIGS. 29A-B are bar graphs depicting the change in nitrite concentrations (FIG. 29A) and AChE activity (FIG. 29B) following LPS, CpG1826 or BW284c51 administration in murine RAW 264.7 macrophage-derived cell line.

FIG. 30A is a bar graph depicting that the increase in miR-132 is specific to LPS challenge in primary human macrophages.

FIG. 30B is a bar graph depicting the change in ACHE mRNA levels following LPS challenge in RAW 264.7 cells, 24 hours following treatment.

FIG. 31A is a graph depicting the kinetics of LPS effects of RAW 264.7 cells.

FIG. 31B is a bar graph depicting that LPS specifically up-regulates miR-132 in human macrophages.

FIG. 32 is a bar graph illustrating that microRNAs 132, 182* and 212 are consistently up-regulated following TLR4 challenge in human primary cultured macrophages as assayed by RTPCR analysis.

FIGS. 33A-B are photomicrographs illustrating the expression of microRNA 132 in the cytoplasm of activated primary macrophages. Red labeling in FIG. 33B shows the nuclei.

FIG. 34 is a bar graph depicting the percentage of miRNAs significantly changed by immunogenic stress. Dark grey represents the number of miRNAs that passed the stringent test for up- or down-regulation. Light grey represents the number of miRNAs that passed the permissive test.

FIG. 35 is a table listing the miRNAs significantly changed in macrophage activation. Listed are miRNAs with a mean LR change of 0.25 or more in absolute value. miRNAs that recurred in different comparisons are marked in colors for ease of location on the table. (Spots where only one of the dyes could be detected were omitted for the stringent test but included in the permissive; thus the calculated LR values of the permissive analysis are meaningless, but the trend indications may be more comprehensive than in the stringent analysis.)

FIG. 36 is a covariance of microRNA profile following macrophage activation similarities between EN101 and CpG reactions.

FIG. 37 is a table covering the outcome of the comparisons involving acute to chronic stress, short to long and brain regions. CA1=hippocampal CA1, BLA=amygdala. The text of the submitted report details the results.

FIG. 38 is a graph showing miR203 and 134 as outliers between the mouse amygdale and the rat CEA. Thus, prolonged stress upregulated 203 in both mouse and rat, while downregulating 134.

FIG. 39 is a bar graph depicting the effect of LNA-modified miRs (132 and 182 SEQ ID NOs. 107 and 108) or control miR (scr) on nitrite concentrations following LPS administration in murine RAW 264.7 macrophage-derived cell line.

FIG. 40 is a bar graph depicting the effect of LNA-modified miR 132 and anti-miR 132 (SEQ ID NOs: 107 and 108, respectively) on AChE-S/R, NOS and beta actin.

FIG. 41 is a schematic model illustrating a pathway which terminates the inflammatory response. A delayed upregulation of miRs suppresses pro-inflammatory factors by binding the AChE mRNA and inhibiting translation. The decrease in AChE activity leads to higher levels of secreted Ach in the cell's environment which in turn acts to suppress inflammation.

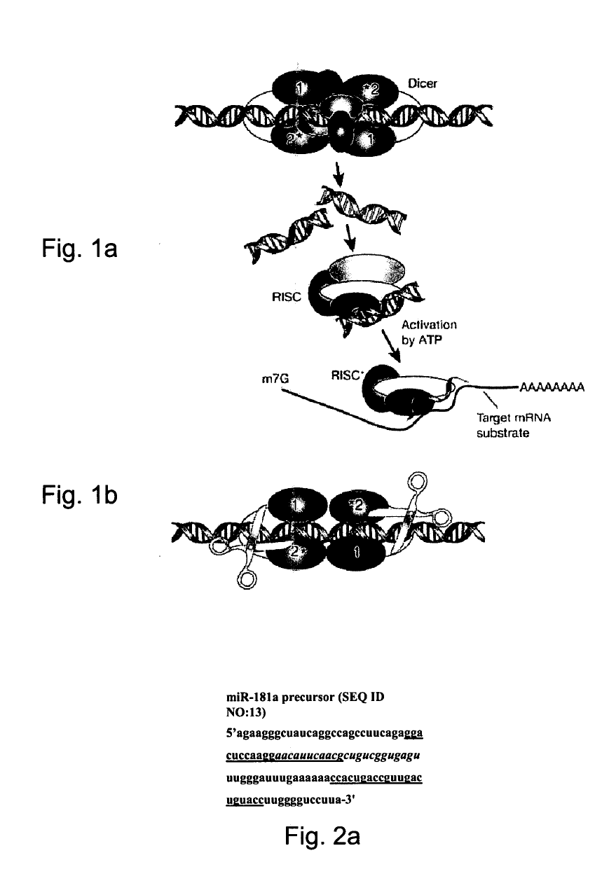

### DESCRIPTION OF THE PREFERRED EMBODIMENTS

The present invention is of isolated polynucleotides, pharmaceutical compositions containing same and methods of using same for treating a myriad of pathologies in which regulating an AChE-associated biological pathway is beneficial. More particularly, the present invention is of isolated polynucleotides, pharmaceutical compositions containing same and methods for regulating the function of a micro-RNA component of an AChE-associated biological pathway, which can be used to regulate an AChE-associated biological pathway, e.g., to shift the ratio between ACHE-S and ACHE-R splice variants/isozymes. Specifically, the present invention can be used to treat various pathologies related to AChE-associated biological pathways and/or pathologies associated with a shift in the ratio between ACHE-S and ACHE-R splice variants/isozymes, such as, but not limited to, apoptosis, a disease in which modulating nitric oxide levels is therapeutically beneficial, aberrant cholinergic signaling, abnormal hematopoietic proliferation and/or differentiation, cellular stress, exposure to inflammatory response-inducing agents, and/or exposure to organophosphates or to dopaminergic neurotoxin, Alzheimer's disease (AD), Myasthenia gravis, various cancer tumors such as glioblastoma, lung cancer (e.g., small cell lung carcinoma), non-Hodgkin's lymphoma and astrocyte tumors, stress disorders such as post-traumatic stress disorder (PTSD), male infertility, behavioral impairment, enhanced fear memory and/or long-term potentiation.

The principles and operation of the agents and methods according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Micro-RNA are small 20- to 24-nucleotide (nt) RNA molecules members of the family of non-coding small RNAs. Micro-RNAs were identified in mammals, worms, fruit flies and plants and are believed to regulate the stability of their target messenger RNA (mRNA) transcripts in a tissue- and cell type-specific manner. The proposed mechanism of their regulation is either via binding to the 3′-untranslated region (3′-UTR) of target mRNAs and thereby suppressing translation, or in similar manner to siRNAs, by binding to and destroying target transcripts in a sequence-dependent manner. Micro-RNA were found to be involved in various cell differentiation pathways including modulation of hematopoiesis [Chen, 2004 (Supra)], differentiation of human neural progenitor NT2 cells [Kawasaki and Taira, 2003a (Supra)] and differentiation of adipocyte (Esau C, et al., 2004, J. Biol. Chem. 279: 52361-5). In addition, micro-RNA were implicated in various neurological diseases such as Fragile X syndrome, spinal muscular atrophy (SMA), early onset parkinsonism (Waisman syndrome) and X-linked mental retardation (MRX3)] as well as in precancerous and cancerous pathologies such as Wilm's tumor, testicular germ cell tumor, chronic lymphocytic leukemia (CLL), B cell leukemia, precancerous and neoplastic colorectal tissues and Burkkit's lymphoma. Moreover, intron-derived micro-RNA-like molecules (Id-micro-RNA) were suggested as tools for analysis of gene function and development of gene-specific therapeutics [Lin and Ying, 2004b (Supra)].

The various biological functions of micro-RNAs were further demonstrated using antisense oligonucleotides directed against various micro-RNAs. For example, 2′-O-methyl oligoribonucleotides directed against the miR-21 micro-RNA resulted in reversal of EGFP expression in HeLa cells transformed to express exogenous EGFP siRNA (Meister G, et al., 2004, RNA 10: 544-550). In addition, 2′-O-methylated oligos directed against the let-7 micro-RNA of C. elegans were shown to suppress the effect of an exogenous let-7 micro-RNA assembled to the RISC complex [Hutvagner G, 2004 (Supra)]. Moreover, specific inhibition of miR-143 micro-RNA using an antisense oligonucleotide resulted in inhibition of adipocyte differentiation [Esau C, 2004, (Supra)]. However, the extracellular signals inducing changes in miRNA levels and mode of functioning remained obscure. More specifically, the involvement and function of micro-RNA components in AChE-related biological pathways have not been studied yet. Because cholinergic signaling provides the link between the immune and the nervous system (Tracey, 2002) and since it controls mammalian stress reactions (Meshorer et al., 2002, Kaufer et al., 1998), this invention teaches universal concepts referring to these organismal reactions and how they induce cells and tissues to respond to external stress signals of various origins.

While reducing the present invention to practice, the present inventor has uncovered that ACHE associated biological pathways can be regulated by controlling the level of AChE-related micro-RNA (e.g., AChmiRNA, also referred to herein as miRNA-181a).

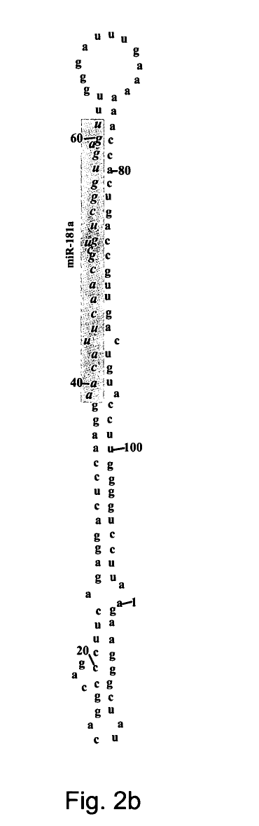

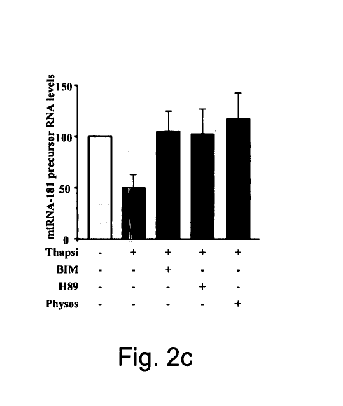

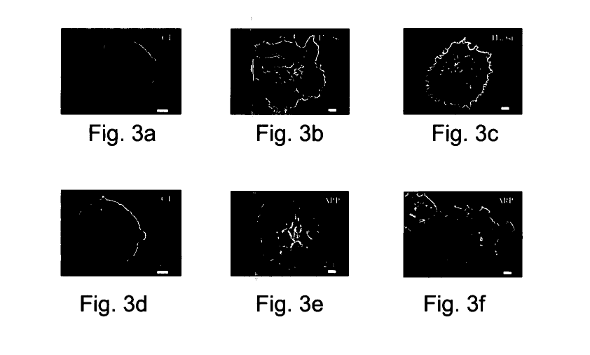

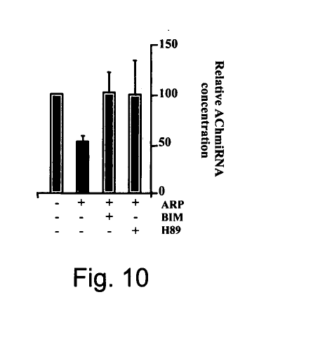

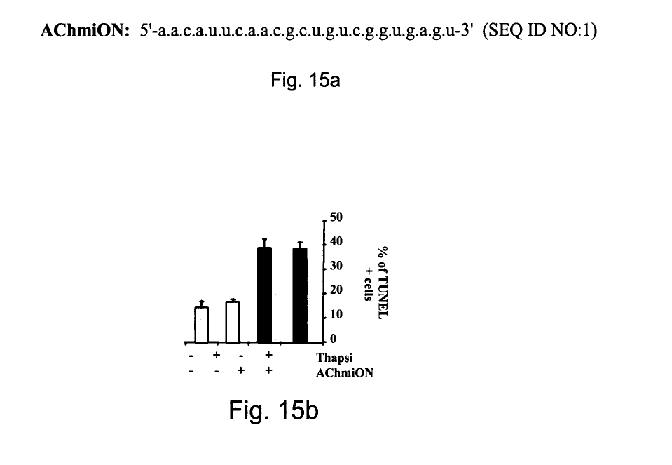

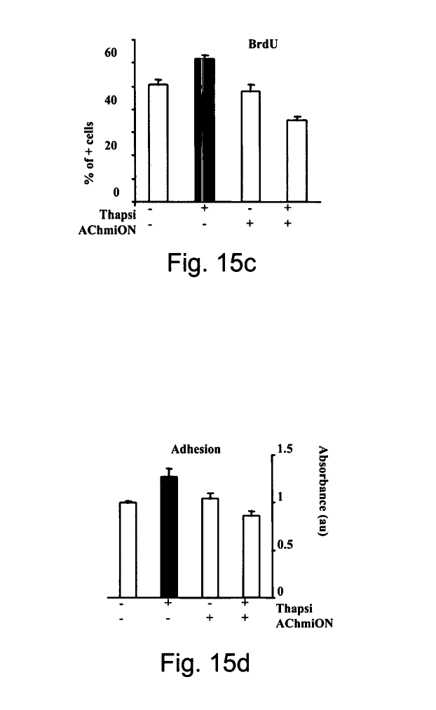

As is described in Example 1 of the Examples section which follows, treatment of the Meg-01 megakaryoblast cells with Thapsigargin (which induces ER-calcium release) resulted in a decrease of AChmiRNA level (precursor—SEQ ID NO:13, amplicon—SEQ ID NO:14) (FIG. 2c) and enhancement of megakaryocyte differentiation and maturation (FIGS. 3a-c, 4b and e and 5b). In addition, treatment of Meg-01 cells with ARP, a synthetic peptide mimicking the C terminal peptide of hAChE-R (SEQ ID NO:3) resulted in a similar decrease in the level of AChmiRNA (FIG. 10) and induction of megakaryocyte differentiation and maturation (FIGS. 3d-f, 4c and e and 5a). Moreover, as is described in Example 3 of the Examples section which follows, treatment of Meg-01 cells with a synthetic 2-O-methylated RNA oligonucleotide (AChmiON; SEQ ID NO:23) resulted in an increase in the level of DNA fragmentation as detected by the TUNEL assay (FIG. 15b), demonstrating increased level of apoptosis.

Additionally, as described in Example 6, the cholinergic system and the TLR (toll like receptor) system of pathogen recognition are causally interrelated. Stimulation of TLRs induced an increase in AChmiRNA levels. This relationship is corroborated by the fact that both stimulation of TLRs and addition of the synthetic AchmiRNA (AChmiON; SEQ ID NO:23) induced an increase in nitric oxide levels as described in Example 7.

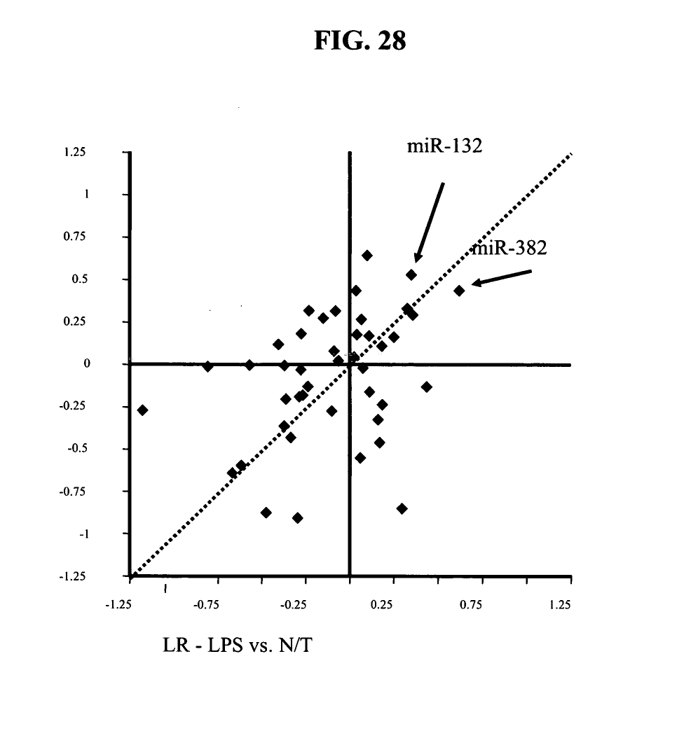

Whilst further reducing the invention to practice the present inventors have shown by microarray analysis that various miRNAs are altered under stress conditions, such conditions being integrally related to the AChE pathway. Specifically two of these miRNAs—132 and 182* are both predicted to be complementary to AChE and were shown to be up-regulated by endotoxin (FIGS. 28-32). The up-regulation of these miRNAs was accompanied by a down-regulation of ACHE activity.

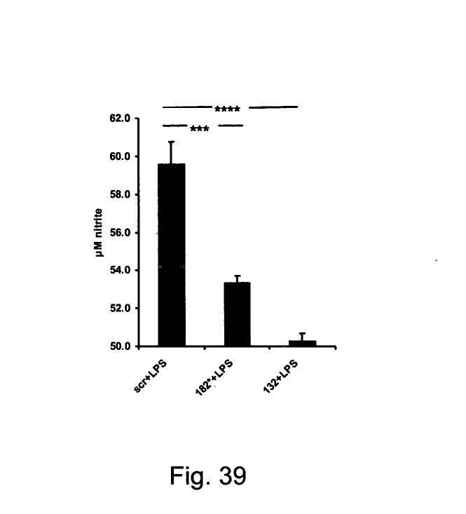

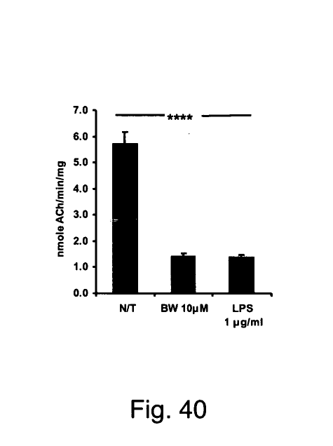

The ability of the miRNA sequences of the present invention to modulate AChE-mediated inflammation was further demonstrated by treatment of stimulated macrophages (murine and human, Examples 11-12 respectively) with miR mimetics. As shown in FIG. 39, LNA-modified miRs downregulated inflammation (as evidenced by nitrite concentrations) following LPS treatment of murine RAW 264.7 macrophage-derived cell line. This effect was shown to be ACHE dependent as demonstrated in FIG. 40 showing the effect of miR 132 on AChE levels.

These findings enqeuivocally support a therapeutic value for the oligonucleotides of the present invention.

Thus, according to one aspect of the present invention there is provided a method of regulating an AChE-associated biological pathway having a miRNA component. The method of this aspect of the present invention is effected by subjecting the AChE-associated biological pathway to an agent capable of regulating a function of the miRNA, thereby regulating the AChE-associated biological pathway.

The term “AChE” as used herein encompasses both the gene coding acetylcholinesterase (AChE), the RNA transcripts encoded by the ACHE gene (i.e., alternatively spliced RNA molecules) and the various isoforms of the AChE protein (EC 3.1.1.7, GenBank Accession No. P22303; ACES_HUMAN).

The phrase “AChE-associated biological pathway” refers to any biological pathway which involves, is regulated by, stimulated by, and/or results from acetylcholinesterase (AChE). Non-limiting examples of such biological pathways include various cholinergic signaling pathways and cross-signaling pathways (e.g., NO), embryonic development, nervous system development, retina development, neoplasma, neurodegeneration, hematopoiesis, megakaryocyte proliferation and/or differentiation, neuronal cell differentiation, apoptosis, stress reactions and immune reaction. See for example, Johnson G and Moore S W, 2000, Int. J. Dev. Neurosci. 18: 781-90; Cheon E W and Saito T, 1999, Brain Res. Dev. Brain Res. 116: 97-109; Deutsch V R, et al., 2002, Exp. Hematol. 30: 1153-61; Jin Q H, et al., 2004, Acta. Pharmacol. Sin. 25: 1013-21; Huang X, et al., 2005, Cell Cycle, January 19; 4(1) [Epub ahead of print]; Park S E, et al., 2004, Cancer Res. 64: 2652-5; Erratum in: Cancer Res. 2004, 64: 9230, which are fully incorporated herein by reference.

The phrase “miRNA component” refers to micro-RNA molecules. Micro-RNAs are processed from pre-miR (pre-micro-RNA precursors). Pre-miRs are a set of precursor miRNA molecules transcribed by RNA polymerase III that are efficiently processed into functional miRNAs, e.g., upon transfection into cultured cells. A Pre-miR can be used to elicit specific miRNA activity in cell types that do not normally express this miRNA, thus addressing the function of its target by down regulating its expression in a “gain of (miRNA) function” experiment. Pre-miR designs exist to all of the known miRNAs listed in the miRNA Registry and can be readily designed for any research.

According to this aspect of the present invention, the micro-RNA component of the present invention is part of, involved in and/or associated with an AChE-associated pathway. Such a micro-RNA can be identified via various databases including for example the micro-RNA registry (http://www.sanger.ac.uk/Software/Rfam/mirna/index.shtml). According to one embodiment the miRNA of the present invention is set forth by SEQ ID NO:21, 22 and/or 23. According to another embodiment the miRNA of the present invention is set forth by SEQ ID NOs: 54, 93, 94, 98, 99, 100, 107 and 108.

According to yet another embodiment the miRNA of the present invention is set forth by SEQ ID NOs: 25-100 as listed in Table 1 hereinbelow.

TABLE 1

| | | |
|---|---|---|
| Seq id no: | MiR no: | Sequence: |

| | | |
|---|---|---|
| 25 | 29b | uagcaccauuugaaaucaguguu |
| 26 | 201 | uacucaguaaggcauuguucu |
| 27 | 293 | agugccgcagaguuuguagugu |
| 28 | 30a-5p | uguaaacauccucgacuggaag |
| 29 | 17-3p | acugcagugaaggcacuugu |
| 30 | 291-5p | caucaaaguggaggcccucucu |
| 31 | 298 | ggcagaggagggcuguucuucc |
| 32 | 294 | aaagugcuucccuuuugugugu |
| 33 | 17-5p | caaagugcuuacagugcagguagu |
| 34 | 30a-3p | cuuucagucggauguuugcagc |
| 35 | 301-5p | cagugcaauaguauugucaaagc |
| 36 | 292-3p | aagugccgccagguuuugagugu |
| 37 | 146 | ugagaacugaauuccauggguu |
| 38 | 384 | auuccuagaaauuguucaua |
| 39 | 402-a | cuggacuuagggucagaaggcc |
| 40 | 202 | agagguauagggcaugggaaaa |
| 41 | 381 | uauacaagggcaagcucucugu |
| 42 | 16-1 | uagcagcacguaaauauuggcg |
| 43 | 217 | uacugcaucaggaacugauuggau |
| 44 | 361 | uuaucagaaucuccagggguac |
| 45 | 302a | uaagugcuuccauguuuugguga |
| 46 | 183 | uauggcacugguagaauucacug |
| 47 | 1-2 | uggaauguaaagaaguaugua |
| 48 | 302c | uaagugcuuccauguuucagugg |
| 49 | 19a | ugugcaaaucuaugcaaaacuga |
| 50 | 302b | uaagugcuuccauguuuuaguag |
| 51 | 154 | uagguuauccguguugccuucg |
| 52 | 106a | aaaagugcuuacagugcagguagc |
| 53 | 300 | uaugcaagggcaagcucucuuc |
| 54 | 132 | uaacagucuacagccauggucg |
| 55 | 128a | ucacagugaaccggucucuuuu |
| 56 | 340 | uccgucucaguuacuuuauagcc |
| 57 | 293 | agugccgcagaguuuguagugu |
| 58 | 129-2 | cuuuuugcggucugggcuugc |
| 59 | 423 | agcucggucugaggccccucag |
| 60 | 382 | gaaguuguucgugguggauucg |
| 61 | 133a-1 | uugguccccuucaaccagcugu |
| 62 | 411 | aacacgguccacuaacccucagu |
| 63 | 199a-2 | cccaguguucagacuaccuguuc |
| 64 | 330 | gcaaagcacacggccugcagaga |
| 65 | 27a | uucacaguggcuaaguuccgc |
| 66 | 410 | aauauaacacagauggccugu |
| 67 | 95 | uucaacggguauuuauugagca |
| 68 | 148a | ucagugcacuacagaacuuugu |
| 69 | 93 | aaagugcuguucgugcagguag |
| 70 | 185 | uggagagaaaggcaguuc |
| 71 | 17-5p | caaagugcuuacagugcagguagu |
| 72 | 33 | gugcauuguaguugcauug |
| 73 | 9 | ucuuugguuaucuagcuguauga |
| 74 | 9* | uaaagcuagauaaccgaaagu |
| 75 | 219 | ugauuguccaaacgcaauucu |
| 76 | 301 | cagugcaauaguauugucaaagc |
| 77 | 221 | agcuacauugucugcuggguuu |
| 78 | 145 | guccaguuuucccaggaaucccuu |
| 79 | 122a | uggagugugacaaugguguuugu |
| 80 | 140 | cagugguuuuacccuaugguag |
| 81 | 26a | uucaaguaauccaggauaggc |
| 82 | 195 | uagcagcacagaaauauuggc |
| 83 | 376b | aucauagaggaacauccacuuu |
| 84 | 215 | augaccuaugaauugacagac |
| 85 | 147 | guguguggaaaugcuucugc |
| 86 | 372 | aaagugcugcgacauuugagcgu |
| 87 | 335 | ucaagagcaauaacgaaaaaugu |
| 88 | 153 | uugcauagucacaaaaguga |
| 89 | 425 | aucgggaaugucguguccgcc |
| 90 | 24 | uggcucaguucagcaggaacag |
| 91 | 130b | cagugcaaugaugaaagggcau |
| 92 | 155 | uuaaugcuaaucgugauagggg |
| 93 | 182* | ugguucuagacuugccaacua |
| 94 | 212 | uaacagucuccagucacggcc |
| 95 | 32 | uauugcacauuacuaaguugc |
| 96 | 214 | acagcaggcacagacaggcag |
| 97 | 203 | gugaaauguuuaggaccacuag |
| 98 | 28 | aaggagcucacagucuauugag |
| 99 | 125a | ucccugagacccuuuaaccugug |
| 100 | 125b | ucccugagacccuaacuuguga |

As used herein, the phrase “function of the miRNA” relates to binding, attaching, regulating, processing, interfering, augmenting, stabilizing and/or destablizing a miRNA target, i.e., the target that is regulated by the action and/or presence of the micro-RNA. Such a target can be any molecule, including, but not limited to, DNA molecules, RNA molecules and polypeptides (e.g., polypeptides which are part of the RISC complex preferably RNA molecules). Preferably, such a target is an RNA molecule.

According to preferred embodiments of the present invention regulating can be upregulating (i.e., increasing) or downregulating (i.e., decreasing) the function of the miRNA of the present invention.

The agents of the present invention can be any molecule effective for its intended use, including, but not limited to, chemicals, antibiotic compounds known to modify gene expression, modified or unmodified polynucleotides (including oligonucleotides), polypeptides, peptides, small RNA molecules, micro-RNAs and anti-micro-RNAs. Preferably, the agent used by the present invention is a polynucleotide.

The term “polynucleotide” refers to a single-stranded or double-stranded oligomer or polymer of ribonucleic acid (RNA), deoxyribonucleic acid (DNA) or mimetics thereof. This term includes polynucleotides and/or oligonucleotides derived from naturally occurring nucleic acids molecules (e.g., RNA or DNA), synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to respective naturally occurring portions.

The length of the polynucleotide of the present invention is optionally of 100 nucleotides or less, optionally of 90 nucleotides or less, optionally 80 nucleotides or less, optionally 70 nucleotides or less, optionally 60 nucleotides or less, optionally 50 nucleotides or less, optionally 40 nucleotides or less, optionally 30 nucleotides or less, e.g., 29 nucleotides, 28 nucleotides, 27 nucleotides, 26 nucleotides, 25 nucleotides, 24 nucleotides, 23 nucleotides, 22 nucleotides, 21 nucleotides, 20 nucleotides, 19 nucleotides, 18 nucleotides, 17 nucleotides, 16 nucleotides, 15 nucleotides, optionally between 12 and 24 nucleotides, optionally between 5-15, optionally, between 5-25, most preferably, about 20-25 nucleotides.

The polynucleotides (including oligonucleotides) designed according to the teachings of the present invention can be generated according to any oligonucleotide synthesis method known in the art, including both enzymatic syntheses or solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

It will be appreciated that a polynucleotide comprising an RNA molecule can be also generated using an expression vector as is further described hereinbelow.

Preferably, the polynucleotide of the present invention is a modified polynucleotide. Polynucleotides can be modified using various methods known in the art.

For example, the oligonucleotides or polynucleotides of the present invention may comprise heterocylic nucleosides consisting of purines and the pyrimidines bases, bonded in a 3′-to-5′ phosphodiester linkage.

Preferably used oligonucleotides or polynucleotides are those modified either in backbone, internucleoside linkages, or bases, as is broadly described hereinunder.

Specific examples of preferred oligonucleotides or polynucleotides useful according to this aspect of the present invention include oligonucleotides or polynucleotides containing modified backbones or non-natural internucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Oligonucleotides or polynucleotides of the present invention may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications.

According to preferred embodiments of the present invention the modified polynucleotide of the present invention is partially 2′-oxymethylated, or more preferably, is fully 2′-oxymethylated (see for example the polynucleotide set forth by SEQ ID NO:23 and SEQ ID NO:24).

According to preferred embodiments of the present invention, upregulating the function of the miRNA of the present invention is effected using a polynucleotide which comprises at least 10 consecutive nucleotides of the nucleic acid sequence set forth by SEQ ID NO:1, more preferably, at least 11, more preferably, at least 12, more preferably, at least 13, more preferably, at least 14, more preferably, at least 15, more preferably, at least 16, more preferably, at least 17, more preferably, at least 18, more preferably, at least 19, more preferably, at least 20, more preferably, at least 21, more preferably, most preferably, at least 22 consecutive nucleotides from the nucleic acid sequence set forth by SEQ ID NO:1.

Preferably, upregulating the function of the miRNA of the present invention is effected using a polynucleotide which is hybridizable in cells under physiological conditions to an RNA molecule which comprises a nucleic acid sequence as set forth in SEQ ID NO:2. Non-limiting examples of such polynucleotides include the polynucleotides set forth by SEQ ID NO:1 or 23.

As used herein, the term “hybridizable” refers to capable of hybridizing, i.e., forming a double strand molecule such as RNA:RNA, RNA:DNA and/or DNA:DNA molecules. “Physiological conditions” refer to the conditions present in cells, tissue or a whole organism, or body. Preferably, the physiological conditions used by the present invention include a temperature between 34-40° C., more preferably, a temperature between 35-38° C., more preferably, a temperature between 36 and 37.5° C., most preferably, a temperature between 37 to 37.5° C.; salt concentrations (e.g., sodium chloride NaCl) between 0.8-1%, more preferably, about 0.9%; and/or pH values in the range of 6.5-8, more preferably, 6.5-7.5, most preferably, pH of 7-7.5.

According to presently preferred embodiments, upregulating the function of the miRNA of the present invention is effected using a polynucleotide having a nucleic acid sequence as set forth in SEQ ID NO:1 (e.g., the polynucleotide set forth by SEQ ID NO:23).

Since as is mentioned hereinabove and is shown in the Examples section which follows, micro-RNAs are processed molecules derived from specific precursors (i.e., pre-miRNA), upregulation of a specific miRNA function can be effected using a specific miRNA precursor molecule.

According to preferred embodiments of the present invention, upregulating the function of the miRNA of the present invention is effected using a polynucleotide which comprises at least 25 consecutive nucleotides of the nucleic acid sequence set forth in SEQ ID NO:13, more preferably, at least 30, more preferably, at least 35, more preferably, at least 40, more preferably, at least 45, more preferably, at least 50, more preferably, at least 55, more preferably, at least 60, more preferably, at least 65, more preferably, at least 70, more preferably, at least 75, more preferably, at least 80, more preferably, at least 85, more preferably, at least 90, more preferably, at least 95, more preferably, at least 100, more preferably, at least 105, most preferably, at least 109 consecutive nucleotides of the nucleic acid sequence set forth in SEQ NO: 13.

Upregulating the function of the miRNA of the present invention can also be effected using a polynucleotide which comprises at least 20 consecutive nucleotides from the nucleic acid sequence set forth by SEQ ID NO:13 and/or at least 10 consecutive nucleotides of SEQ ID NO:1, optionally, at least 25 consecutive nucleotides from the nucleic acid sequence set forth by SEQ ID NO:13 and/or at least 15 consecutive nucleotides of SEQ ID NO:1, optionally, at least 30 consecutive nucleotides from the nucleic acid sequence set forth by SEQ ID NO:13 and/or at least 20 consecutive nucleotides of SEQ ID NO:1, optionally, at least 30 consecutive nucleotides from the nucleic acid sequence set forth by SEQ ID NO:13 and/or at least 24 consecutive nucleotides of SEQ ID NO:1.

For example, since the AChmiRNA molecule (natural—SEQ ID NO:21; synthetic—SEQ ID NO:1) is derived from the pre-AChmiRNA molecule (natural—SEQ ID NO:22; synthetic—SEQ ID NO:13), upregulating the function of AChmiRNA can be effected using a polynucleotide capable of producing a functional AChmiRNA (e.g., a polynucleotide having nucleic acid sequence as set forth in SEQ ID NO:13).

Thus, according to presently preferred embodiments of the present invention, upregulating the function of the miRNA of the present invention is effected using a polynucleotide as set forth by SEQ ID NO:13.

Downregulating the function of the miRNA of the present invention can be effected using a polynucleotide which comprises at least 10 consecutive nucleotides of the nucleic acid sequence set forth in SEQ ID NO:2, optionally, at least 11, optionally, at least 12, optionally, at least 13, optionally, at least 14, optionally, at least 15, optionally, at least 16, optionally, at least 17, optionally, at least 18, optionally, at least 19, optionally, at least 20, optionally, at least 21, preferably, at least 22 consecutive nucleotides of the nucleic acid sequence set forth in SEQ ID NO:2.

Downregulating the function of the miRNA of the present invention can also be effected using a polynucleotide which is hybridizable in cells under physiological conditions to an RNA molecule which comprises a nucleic acid sequence as set forth by SEQ ID NO:21 and/or 22. A non-limiting example of such polynucleotide is the polynucleotide set forth by SEQ ID NO:2. Hence, downregulating the function of the miRNA of the present invention can be effected using a polynucleotide as set forth by SEQ ID NO:2.

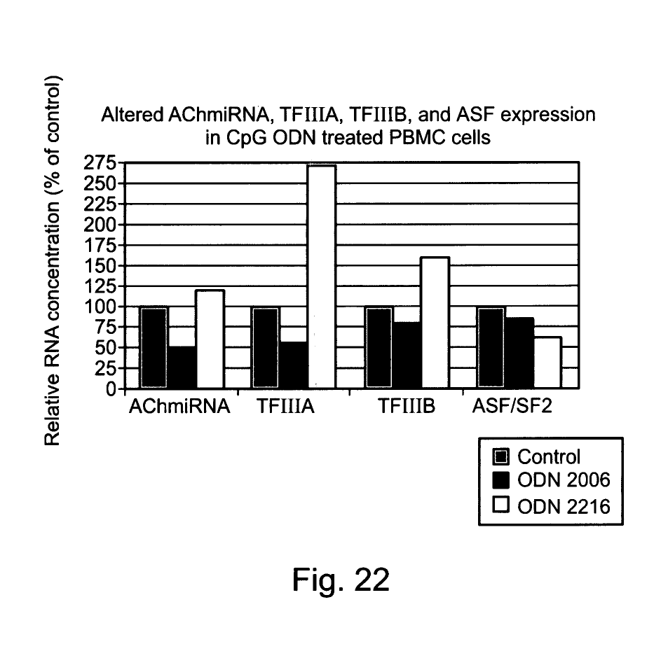

As is shown in FIGS. 21 and 22 and is described in Example 6 of the Examples section which follows, the level of AChmiRNA (SEQ ID NO:21; amplicon—SEQ ID NO:14) was significantly increased in peripheral blood monocyte cells (PBMC) which were stimulated with the TLR9 ligand, CpG-A oligonucleotide 2216 (SEQ ID NO:12). On the other hand, the level of AChmiRNA was decreased in PBMC cells which were treated with the CpG ODN 2006 (SEQ ID NO:19) which exhibit reciprocal effects on innate immune response.

Thus, according to embodiments of the present invention, upregulating the function of the miRNA of the present invention can be effected by a polynucleotide as set forth by SEQ ID NO:12 or a functional homolog thereof.

As used herein, the phrase “functional homolog” refers to any molecule or agent capable of exerting the function of a reference molecule, e.g., the polynucleotide set forth by SEQ ID NO:12, i.e., in this case, stimulating the immune response, preferably via the toll-like receptor (TLR) pathway.

On the other hand, downregulating the function of the miRNA of the present invention can be effected using a polynucleotide as set forth by SEQ ID NO:19 or a functional homolog thereof (i.e., a molecule or agent capable of downregulating the immune response).

The correlation between the decrease in AChmiRNA level (as detected by RT-PCR using the amplicon set forth by SEQ ID NO:14) and the increased differentiation and maturation of the megakaryoblast cells demonstrated in FIGS. 2c, 3a-f, 4a-e, 5a-b and 10 and the Examples section which follows, indicate that agents capable of regulating micro-RNA function can be used to alter differentiation and/or proliferation of hematopoietic cells.

Thus, according to yet another aspect of the present invention there is provided a method of altering differentiation and/or proliferation of hematopoietic progenitor and/or stem cells. The method according to this aspect of the present invention is effected by subjecting the progenitor and/or stem cells to an agent capable of regulating a function of a miRNA component of an AChE-associated biological pathway in the progenitor and/or stem cells, thereby altering differentiation and/or proliferation of the hematopoietic progenitor and/or stem cells.

As used herein, the phrase “progenitor and/or stem cells” refers to cells which are capable of differentiating into other cell types having a particular, specialized function (i.e., “fully differentiated” cells) or remaining in an undifferentiated state hereinafter “pluripotent stem cells”. Hematopoietic stem and/or progenitor cells are capable of differentiation into the myeloid or lymphoid cell lineages. The myeloid cell lineage includes eosinophils, basophils, neutrophils, monocytes, macrophages, megakaryoblasts, megakaryocytes (and platelets), as well as erythroblasts and erythrocytes. The lymphoid cell lineage includes T and B lymphocyte cells. Hematopoietic stem and/or progenitor cells can be obtained from bone marrow tissue of an individual at any age, cord blood of a newborn individual, peripheral blood, thymus and/or embryonic stem cells which are induced to differentiate towards the hematopoietic lineage.

The term “altering” as used herein with respect to differentiation and/or proliferation of hematopoietic progenitor and/or stem cells refers to modulating, modifying, or changing the rate (i.e., increasing or decreasing), mode (i.e., differentiation, proliferation or cell death) and/or direction of differentiation (i.e., differentiation into other cell lineages) of the hematopoietic stem and/or progenitor cells.

As used herein, the term “subjecting” with respect to the hematopoietic progenitor and/or stem cells refers to contacting, administering to, providing to, mixing with and/or injecting to the cells or to an organism having the cells any of the agents described herein. Hence, subjecting can be effected in vivo or in vitro.

As is shown in FIG. 15b and is described in Example 3 of the Examples section which follows, administration of AChmiON (SEQ ID NO:23) to megakaryoblast cells (Meg-01) resulted in a significant increase in DNA fragmentation (which is characteristic of apoptosis) either in the presence or absence of Thapsigargin treatment (i.e., with or without calcium-induced megakaryoblast differentiation). These results clearly demonstrate that agents which are capable of regulating the function of micro-RNA (e.g., AChmiON) can be used to regulate apoptosis.

Thus, according to yet an additional aspect of the present invention there is provided a method of regulating apoptosis in cells and/or a tissue of a subject in need thereof. The method according to this aspect of the present invention is effected by subjecting the cells and/or the tissue of the subject to an agent capable of regulating a function of a miRNA component of an AChE-associated biological pathway in the cells and/or tissue, thereby regulating apoptosis in the cells and/or the tissue of the subject.

As used herein, the term “subject” refers to an animal, preferably a mammal, most preferably a human being, including both young and old human beings of both sexes who suffer from or are predisposed to a pathology. The subject according to this aspect of the present invention suffers from a pathology associated with abnormal apoptosis.

As used herein, the phrase “abnormal apoptosis” refers to rate or level of apoptosis (i.e., programmed cell death) which are different (i.e., increased or decreased) from the values present in normal cells, tissues or individuals.

It will be appreciated that abnormal apoptosis can be associated with various pathologies. For example, pathologies associated with reduced level of apoptosis include, but are not limited to, psoriasis (Victor F C and Gottlieb A B, 2002, J. Drugs Dermatol. 1: 264-75), ichthyosis (Melino G, et al., 2000, Methods Enzymol. 322: 433-72), common warts, keratoacanthoma (Tsuji T, 1997, J. Cutan. Pathol. 24: 409-15), seborrhoic keratosis (Satchell A C, et al., 2004, Br. J. Dermatol. 151: 42-9), seborrhea, squamous cell carcinomas (SCC; Seta C, et al., 2000, J. Oral Pathol. Med. 29: 271-8), basal cell carcinoma (BCC; Li C, et al., 2004, Oncogene. 2004, 23: 1608-17), non-melanoma skin cancer (NMSC) and multiple human tumors. In addition, abnormal apoptosis can be associated with exposure to organophosphate inhibitors of AChE used as insecticides which increases the risk of non-Hodgkin's lymphoma (Soreq and Seidman, 2001). On the other hand, pathologies associated with increased level of apoptosis include, but are not limited to, autoimmune diseases (reviewed in Nikitakis N G, et al., 2004, Oral. Surg. Oral. Med. Oral. Pathol. Oral. Radiol. Endod. 97: 476-90), vascular diseases such as atherosclerosis (Kockx M M, Knaapen M W, 2000, J. Pathol. 190: 267-80; Sykes T C, et al., 2001, Eur. J. Vasc. Endovasc. Surg. 22: 389-95), as well as pathologies associated with exposure to anti-AChE poisons which enhance apoptosis in the central nervous system such as of the dopaminergic neurons in the case of Parkinson's disease (BenMoyal-Segal et al., 2005).

As used herein, the phrase “regulating apoptosis” refers to increasing the level and/or rate of apoptosis in cases where a reduced level of apoptosis occurs and decreasing the level and/or rate of apoptosis in cases where an increased level of apoptosis occurs.

The cells and/or the tissue used by the method according to this aspect of the present invention include any type of cells or tissue of the subject. Examples include, but are not limited to, neural cells, retina cells, epidermal cells, hepatocytes, pancreatic cells, osseous cells, cartilaginous cells, elastic cells, fibrous cells, myocytes, myocardial cells, bone marrow cells, endothelial cells, smooth muscle cells, intestinal cells and hematopoietic cells.

It will be appreciated that the cells can be treated in vivo (i.e., inside the organism or the subject) or ex vivo (e.g., in a tissue culture). In case the cells are treated ex vivo, the method preferably includes a step of administering such cells back to the individual (ex vivo cell therapy). In vivo and ex vivo therapies are further discussed hereinbelow.

As mentioned hereinabove, a stimulator (e.g., CpG-A), of an immune response via the toll-like receptor (TLR) pathway upregulates AChmiRNA. Part of the non-specific cellular defense mechanism triggered by CpG-A includes the production of nitric oxide.

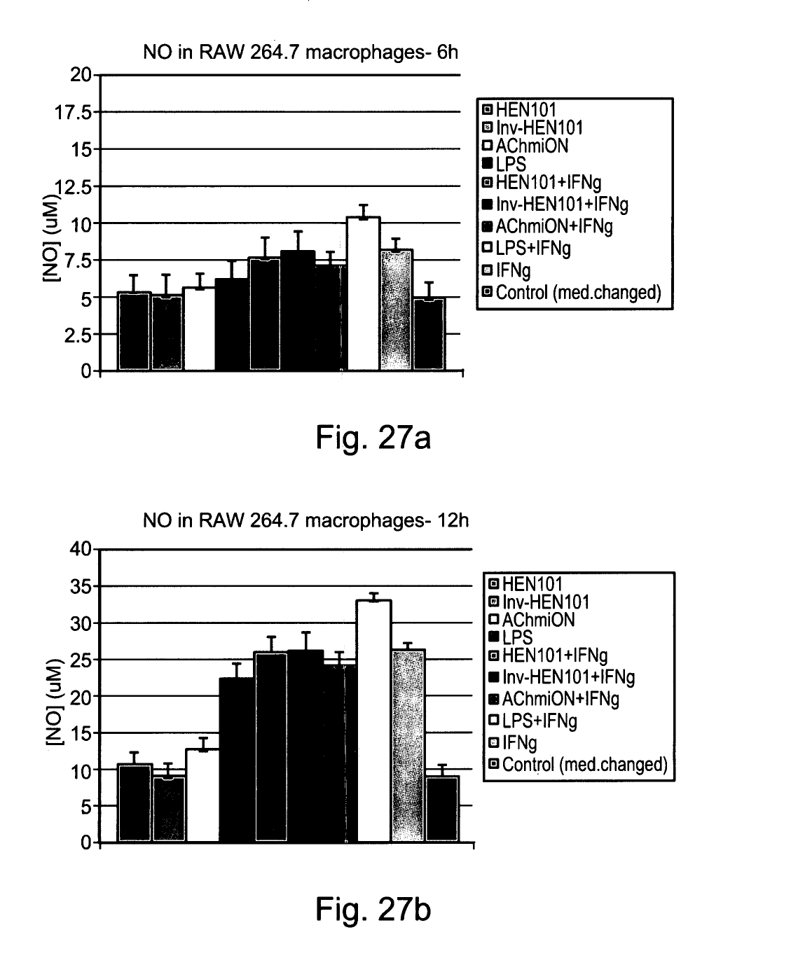

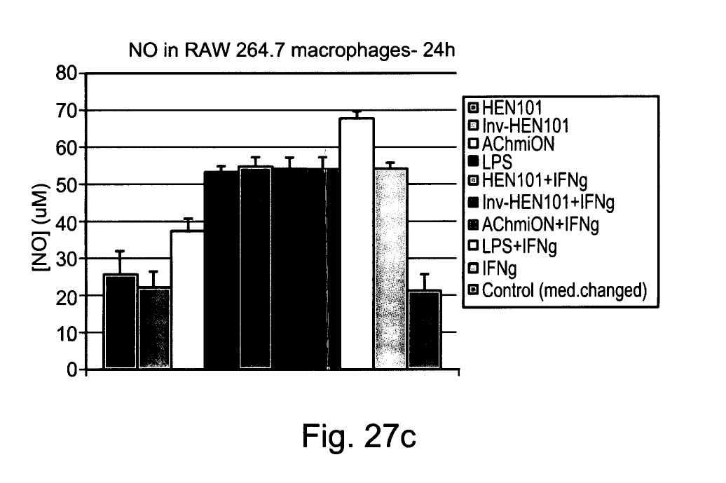

As shown in FIGS. 27a-c and described in Example 7 of the Examples section which follows, administration of AChmiON (SEQ ID NO:23) to murine macrophage RAW 264.7 cells resulted in the production of Nitric Oxide (NO) demonstrating that agents which are capable of regulating the function of micro-RNA (e.g., AChmiON) can also be used to regulate NO levels.

Thus, according to yet an additional aspect of the present invention there is provided a method of treating a disease or condition in which regulating nitric oxide is therapeutically beneficial in a subject, the method comprising administering to a subject in need thereof an agent capable of regulating a miRNA component of an AChE-associated biological pathway.

It will be appreciated that altering NO levels may be therapeutically beneficial for various pathologies as described hereinbelow.

The agent capable of regulating NO may be administered in vivo or ex vivo as discussed hereinbelow.

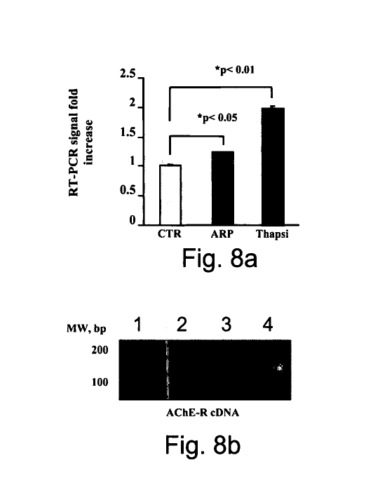

As mentioned hereinabove, induction of megakaryocyte differentiation by either Thapsigargin or ARP treatment was associated with significant decreases in AChmiRNA (FIGS. 2c and 10). Such decreases in AChmiRNA levels were also associated with a splice shift of AChE mRNA transcripts from the synaptic ACHE-S variant (mRNA—SEQ ID NO:15; protein—SEQ ID NO:17) to the readthrough ACHE-R variant (mRNA—SEQ ID NO:16; protein—SEQ ID NO:18) (see FIGS. 8a-b, 9, 10, 12a-b, 16a-d and 24a-b and description in Examples 2 and 3 of the Examples section). These results demonstrate that AChmiRNA regulates splicing of the ACHE gene transcription product.

Thus, according to yet an additional aspect of the present invention there is provided a method of regulating an expression level ratio of AChE-S and ACHE-R and/or ACHE-S mRNA and AChE-R mRNA splice variants in AChE expressing cells. The method according to this aspect of the present invention is effected by subjecting the AChE gene expressing cells to an agent capable of regulating a function of a miRNA component associated with regulating the expression level ratio of AChE-S and ACHE-R splice variants, thereby regulating the expression level of the AChE-S and AChE-R splice variants in the ACHE expressing cells.

As used herein, the term “AChE-R” refers to the ACHE splice variant polypeptide as set forth in SEQ ID NO:18 which results from the readthrough mRNA transcript, AChE-R mRNA as set forth in SEQ ID NO:16.

As used herein, the term “AChE-S” refers to the AChE splice variant polypeptide as set forth in SEQ ID NO:17 which results from the synaptic mRNA transcript, AChE-S mRNA as set forth in SEQ ID NO:15.

The phrase “expression level ratio” refers to the ratio between the expression level of each of the AChE splice variants (i.e., the isoforms AChE-S and AChE-R) at the RNA and/or protein level. It will be appreciated that such a ratio can be determined in cells which express the AChE gene, by measuring the RNA or protein level of each of the variants.

AChE gene expressing cells or ACHE expressing cells, which are interchangeably used herein, can be any cells which express the ACHE gene. Non-limiting examples of such cells can be hematopoietic cells (e.g., red blood cells, megakaryocytes, lymphocytes), neuronal cells, muscle cells, chondrocytes, bone cells, epithelial cells, kidney cells, fibroblasts (e.g., lung fibroblasts), cardiac (heart) cells, and hepatic cells.

While further reducing the present invention to practice the present inventor has uncovered that regulating the function of a micro-RNA can be used to treat pathologies related to AChE-associated biological pathways.

Thus, according to yet another aspect of the present invention there is provided a method of treating a pathology related to an AChE-associated biological pathway. The method according to this aspect of the present invention is effected by administering to a subject in need thereof an agent capable of regulating a function of a miRNA component of the AChE-associated biological pathway, thereby treating the pathology.

The term “treating” refers to inhibiting or arresting the development of a disease, disorder or condition and/or causing the reduction, remission, or regression of a disease, disorder or condition or keeping a disease, disorder or medical condition from occurring in a subject who may be at risk for the disease disorder or condition, but has not yet been diagnosed as having the disease disorder or condition. Those of skill in the art will understand that various methodologies and assays can be used to assess the development of a disease, disorder or condition, and similarly, various methodologies and assays may be used to assess the reduction, remission or regression of a disease, disorder or condition.

The term “pathology” refers to any deviation from a healthy or normal condition, such as a disease, disorder or any abnormal medical condition.

According to one embodiment of the present invention the pathology is characterized by aberrant cholinergic signaling. Such a pathology can be for example, a neurodegenerative disease or disorder such as Alzheimer's disease, Parkinson's disease, Down Syndrome, neurodegeneration in the enteric nervous system (ENS), dementia, Gaucher disease, dementia associated with Lewy bodies, tauopathy disorders and acute and/or chronic neurodegeneration.

According to another embodiment of the present invention the pathology is characterized by abnormal hematopoeitic cell proliferation and/or differentiation. Such a pathology can be for example, myelodysplastic syndrome (MDS), acute myeloid leukemia (AML), refractory anaemia with excess blasts (RAEB), chronic myelomonocytic leukaemia (CMML) and refractory anaemia (RA).

Additionally or alternatively, the pathology is characterized by abnormal megakaryocyte proliferation and/or differentiation, such as thrombocytopenia, idiopathic thrombocytopenic purpura (ITP), congenital amegakaryocytic thrombocytopenia (CAMT), essential thrombocythemia (ET), and acquired amegakaryocytic thrombocytopenia (AATP).

Optionally, the pathology is characterized by cellular stress such as ischemia (Saez-Valero J et al., 2003, Brain Res. Mol. Brain. Res. 117: 240-4) and atherosclerosis (Fuhrman B. et al., 2004, Biochem Biophys Res Commun. 322: 974-8), as well as pathologies characterized by oxidative stress such as vitiligo (Schallreuter K U, et al., 2005; Human epidermal acetylcholinesterase (AChE) is regulated by hydrogen peroxide (HO), Exp Dermatol. 14: 155).

Still optionally, the pathology is caused by drug poisoning such as acute dipterex poisoning (ADP) (Zhou J F et al., 2004, Biomed. Environ. Sci. 17: 223-33).

Alternatively, the pathology is caused by exposure to inflammatory response-inducing agents such as lipopolysaccharide (LPS), Cyclosporin A, PI-88 (Rosenthal M A, et al., 2002, Ann. Oncol. 13: 770-6), Miconazole (Hanada S, et al., 1998, Gen. Pharmacol. 30:791-4), Phospholipase C (PLC) (e.g., from Pseudomonas aeruginosa (Meyers D J, and Berk R S, 1990, Infect. Immun. 58: 659-666), silver nitrate (Brissette L, et al., 1989, J. Biol. Chem. 264: 19327-32) and concanavalin A (Marchal G., et al., 1986, Tubercle 67: 61-7).

Optionally, the pathology is caused by exposure to organophosphates such as those used as insecticides [Chlorphyrifos (CPF), malathion, parathion, diazinon, fenthion, dichlorvos, dimethoate, monocrotophos, phorate, methamidophos, azamethiphos, paraoxon, bis(1-methylethyl) phosphorofluoridate (DFP), dimethyl thiophosphate (DMTP), dimethyl phosphate (DMP), dimethyldithiophosphate (DMDTP), diethyl phosphate (DEP), diethyldithiophosphate (DEDTP), diethylthiophosphate (DETP)], ophthalmic agents (e.g., echothiophate and isofluorophate), antihelmintics agents (e.g., trichlorfon), herbicides [e.g., tribufos (DEF) and merphos], warfare agents (e.g., Tabun, Soman, Sarin and VX) and tricresyl phosphate containing industrial chemicals.

Alternatively, the pathology may be characterized in that modulating (i.e., regulating by up-regulation or down-regulation) nitric oxide levels may be therapeutically beneficial for its treatment. Examples of pathologies in which up-regulating nitric oxide levels may be therapeutically beneficial include but are not limited to angina pectoris (Steven Corwin, M.D., James A. Reiffel, M.D., March, 1985, Arch Intern Med-vol. 145, pp. 538-543), ischemic disease (U.S. Pat. No. 5,278,192), congestive heart failure (Taylor et al., 2004, New England Journal of Medicine, 351:2049-2057) hypertension (U.S. Pat. No. 5,278,192), pulmonary hypertension (U.S. Pat. No. 5,278,192), stroke (U.S. Pat. No. 5,278,192), inflammatory disorder (Dijkstra et al., Scand J Gastroenterol Suppl. 2002; (236):37-41; Chenevier-Gobeux et al., Clinical Science, 2004, 107, 291-296), a bacterial infection, a viral infection, a parasitic infection, an immune disease, a tumor, impotence, hypothermia, abnormal wound healing, a leg ulcer, alopecia and decreased long-term potenetiation.

Examples of pathologies in which down-regulating nitric oxide levels may be therapeutically beneficial include inflammatory disorders (Lamas et al., Trends Pharmacol Sci 1998, 19:436-438; Grisham et al., J Investig Med 2002, 50:272-283), diabetes, neurodegenerative disorders such as Alzheimers (Goodwin et al., Brain Res 1995; 692(1-2):207-14), multiple sclerosis (Bagasra et al., Proc Natl Acad Sci, USA 1995; 92(26):12041-5) and Parkinsons (Hantraye et al., Nat Med 1996; 2(9):1017-21).

As used herein the phrase “inflammatory disorder” includes but is not limited to chronic inflammatory diseases and acute inflammatory diseases. Examples of such diseases and conditions are summarized infra.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med. Sci. 2000 April; 319 (4):234), myasthenic diseases, Lambert-Eaton myasthenic syndrome (Takamori M. Am J Med. Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3): 178); antiphospholipid syndrome (Flamholz R. et al., J Clin Apheresis 1999; 14 (4):171); heart failure, agonist-like beta-adrenoceptor antibodies in heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and, anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al, Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med. Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

It will be appreciated that various pathologies are associated with or characterized by abnormal levels of AChE-S or AChE-R splice variants/isosimes.

Hence, regulating the function of a micro-RNA can be used to treat pathologies related to abnormal levels of ACHE-S or ACHE-R splice variant.

Thus, according to still an additional aspect of the present invention there is provided a method of treating a pathology associated with abnormal levels of ACHE-S or ACHE-R splice variants. The method is effected by administering to a subject in need thereof an agent capable of regulating a function of a miRNA component of an AChE-associated biological pathway, thereby treating the pathology.

For example, abnormally high levels of ACHE-S are found in astrocyte tumor cells (Perry et al., 2001, Oncogen 21: 8428-8441), brains of Alzheimer's disease (AD) patients (Berson A, abstract, in press in the proceedings of the forthcoming AD/PD meeting in Sorrento, Italy, March 2005). According to preferred embodiments of the present invention such pathologies can be treated by reducing the level of AChE-S as described hereinabove.

On the other hand, abnormally high levels of ACHE-R are associated with Myasthenia gravis (MG) (Brenner T., et al., 2003, The FASEB Journal, 17: 214-222), lung cancer (e.g., small cell lung carcinoma) (Karpel R., et al., 1999, Exp. Cell Res. 210: 268-277), stress disorders such as post-traumatic stress disorder (PTSD) (Friedman A, et al., 1996, Nat. Med. 2: 1382-5; Kaufer D, 1998, Nature, 393: 373-7), various cancer tumors such as glioblastoma (Perry C, et al., 2004, Neoplasia 6: 279-286); osteosarcoma (Grisaru D, et al., 1999, Eur J. Biochem. 264: 672-686), male infertility (Mor I, et al., 2001, FASEB 15: 2039041), behavioral impairment (Cohen O, et al., 2002, Mol. Psychiatry. 9: 174-183), enhanced fear memory and/or long-term potentiation (Nijholt, I, et al., 2004, Mol. Psychiatry. 9: 174-183). According to preferred embodiments of the present invention such pathologies can be treated by reducing the level of ACHE-R as described hereinabove.

As mentioned hereinabove, the polynucleotides of the present invention (e.g., an RNA molecule such as those set forth by SEQ ID NO:1, 2 or 13) can be generated using an expression vector.

To express an exogenous polynucleotide (i.e., to produce an RNA molecule) in mammalian cells, a nucleic acid sequence encoding the polynucleotide of the present invention (e.g., SEQ ID NO:1, 2 or 13) is preferably ligated into a nucleic acid construct suitable for mammalian cell expression. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M, et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banedi et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase RNA stability [Soreq et al., 1974; J. Mol Biol. 88: 233-45).

Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRepS, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the polynucleotide of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a TRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of the present invention can also include sequences engineered to enhance stability, production, purification, yield or toxicity of the expressed peptide. For example, the expression of a fusion protein or a cleavable fusion protein comprising Met variant of the present invention and a heterologous protein can be engineered. Such a fusion protein can be designed so that the fusion protein can be readily isolated by affinity chromatography; e.g., by immobilization on a column specific for the heterologous protein. Where a cleavage site is engineered between the Met moiety and the heterologous protein, the Met moiety can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of the present invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by the present invention.

For ex vivo therapy, cells are preferably treated with the agent of the present invention (e.g., an agent which can regulate the function of the micro-RNA), following which they are administered to the subject (individual) which is in need thereof.

Administration of the ex vivo treated cells of the present invention can be effected using any suitable route of introduction, such as intravenous, intraperitoneal, intra-kidney, intra-gastrointestinal track, subcutaneous, transcutaneous, intramuscular, intracutaneous, intrathecal, epidural, and rectal. According to presently preferred embodiments, the ex vivo treated cells of the present invention may be introduced to the individual using intravenous, intra-kidney, intra-gastrointestinal track, and/or intraperitoneal administration.

The cells used for ex vivo treatment according to the present invention can be derived from either autologous sources, such as self bone marrow cells, or from allogeneic sources, such as bone marrow or other cells derived from non-autologous sources. Since non-autologous cells are likely to induce an immune reaction when administered to the body, several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles, and macroencapsulation, involving larger flat-sheet and hollow-fiber membranes (Uludag, H. et al. (2000). Technology of mammalian cell encapsulation. Adv Drug Deliv Rev 42, 29-64).

Methods of preparing microcapsules are known in the art and include for example those disclosed in: Lu, M. Z. et al. (2000). Cell encapsulation with alginate and alpha-phenoxycinnamylidene-acetylated poly(allylamine). Biotechnol Bioeng 70, 479-483; Chang, T. M. and Prakash, S. (2001) Procedures for microencapsulation of enzymes, cells and genetically engineered microorganisms. Mol Biotechnol 17, 249-260; and Lu, M. Z., et al. (2000). A novel cell encapsulation method using photosensitive poly(allylamine alpha-cyanocinnamylideneacetate). J Microencapsul 17, 245-521.

For example, microcapsules are prepared using modified collagen in a complex with a ter-polymer shell of 2-hydroxyethyl methylacrylate (HEMA), methacrylic acid (MAA), and methyl methacrylate (MMA), resulting in a capsule thickness of 2-5 μm. Such microcapsules can be further encapsulated with an additional 2-5 μm of ter-polymer shells in order to impart a negatively charged smooth surface and to minimize plasma protein absorption (Chia, S. M. et al. (2002). Multi-layered microcapsules for cell encapsulation. Biomaterials 23, 849-856).

Other microcapsules are based on alginate, a marine polysaccharide (Sambanis, A. (2003). Encapsulated islets in diabetes treatment. Diabetes Thechnol Ther 5, 665-668), or its derivatives. For example, microcapsules can be prepared by the polyelectrolyte complexation between the polyanions sodium alginate and sodium cellulose sulphate and the polycation poly(methylene-co-guanidine) hydrochloride in the presence of calcium chloride.

It will be appreciated that cell encapsulation is improved when smaller capsules are used. Thus, for instance, the quality control, mechanical stability, diffusion properties, and in vitro activities of encapsulated cells improved when the capsule size was reduced from 1 mm to 400 μm (Canaple, L. et al. (2002). Improving cell encapsulation through size control. J Biomater Sci Polym Ed 13, 783-96). Moreover, nanoporous biocapsules with well-controlled pore size as small as 7 nm, tailored surface chemistries, and precise microarchitectures were found to successfully immunoisolate microenvironments for cells (See: Williams, D. (1999). Small is beautiful: microparticle and nanoparticle technology in medical devices. Med Device Technol 10, 6-9; and Desai, T. A. (2002). Microfabrication technology for pancreatic cell encapsulation. Expert Opin Biol Ther 2, 633-646).

The agent, the polynucleotide and/or the expression vector of the present invention can be administered to the individual per se or as part of a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the agent, the polynucleotide and/or the expression vector of the present invention accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intracardiac, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., the agent, the polynucleotide and/or the expression vector of the present invention) effective to prevent, alleviate, or ameliorate symptoms of the pathology [e.g., a pathology related to an AChE-associated biological pathway such as thrombocytopenia, idiopathic thrombocytopenic purpura (ITP), congenital amegakaryocytic thrombocytopenia (CAMT), essential thrombocythemia (ET), acquired amegakaryocytic thrombocytopenia (AATP)] or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

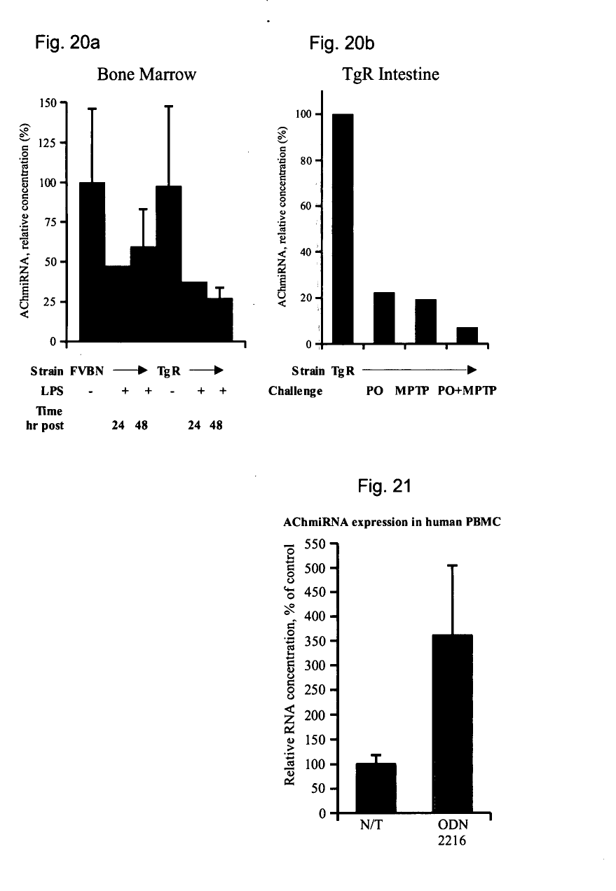

As mentioned hereinabove, the level of AChmiRNA was reduced following the induction of megakaryocyte differentiation and maturation. In addition, as shown in FIGS. 20a-b, 21 and 22 and described in Example 6 of the Examples section which follows, the level of AChmiRNA was reduced in bone marrow and intestine of mice exposed to paraoxon (a cholinesterase inhibitor), MPTP (a dopaminergic poison) or LPS (an immunological insult). In addition, the level of AChmiRNA was significantly increased in human peripheral blood monocyte cells (PBMC) subjected to the TLR-9 ligand [CPG-A ODN 2216 (SEQ ID NO:12)] and, conversely, the level of AChmiRNA was significantly decreased in PBMC subjected to ODN 2206 (SEQ ID NO:19) having a reciprocal effect on innate immune response.

While further reducing the present invention to practice, the present inventor has uncovered that the level of a micro-RNA component of an AChE-associated biological pathway can be used as a diagnostic marker for various pathologies associated with such a micro-RNA.

Thus, according to yet a further aspect of the present invention there is provided a method of diagnosing a pathology associated with abnormal function of a miRNA component of an AChE-associated biological pathway in a subject. The method according to this aspect of the present invention is effected by obtaining a biological sample from the subject and determining a level of the miRNA in cells of the biological sample, wherein a level of the miRNA above or below a predetermined threshold or range is indicative of a presence of a pathology associated with abnormal function of the miRNA.

As used herein the term “diagnosing” refers to classifying a pathology (e.g., a disease, disorder, syndrome, medical condition and/or a symptom thereof), determining a severity of the pathology, monitoring the progression of a pathology, forecasting an outcome of the pathology and/or prospects of recovery (e.g., prognosis).

As used herein “a biological sample” refers to a sample of tissue or fluid derived from a subject, including, but not limited to, for example, blood, plasma, serum, spinal fluid, lymph fluid, the external sections of the skin, respiratory, intestinal, and genitourinary tracts, tears, saliva, sputum, milk, blood cells, bone marrow, cord blood, tumors, neuronal tissue, organs, and also samples of in vivo cell culture constituents. It should be noted that such a biological sample may also optionally comprise a sample that has not been physically removed from the subject as described in greater detail below.

As used herein, the term “level” refers to expression levels of the miRNA molecule or its precursor used in context of the present invention (e.g., the miRNA set forth by SEQ ID NO:21 or 22).

Typically the level of the micro-RNA in a biological sample obtained from the subject is different (i.e., increased or decreased) from the level of the same variant in a similar sample obtained from a healthy individual or the average of a plurality of individuals.

As used herein the “predetermined threshold and/or range” is calculated based on the level detected in biological samples obtained from at least two individuals who do not suffer from the pathology.

Numerous well-known tissue or fluid collection methods can be utilized to collect the biological sample from the subject in order to determine the level of the miRNA in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Detection of the level of the miRNA can be effected using various methods known in the art, including RNA-based hybridization methods (e.g., Northern blot hybridization, RNA in situ hybridization and chip hybridization) and reverse transcription-based detection methods (e.g., RT-PCR, quantitative RT-PCR, semi-quantitative RT-PCR, real-time RT-PCR, in situ RT-PCR, primer extension, mass spectroscopy, sequencing, sequencing by hybridization, LCR (LAR), Self-Sustained Synthetic Reaction (3SR/NASBA), Q-Beta (Qb) Replicase reaction, cycling probe reaction (CPR), a branched DNA analysis, and detection of at least one nucleic acid change).

Following is a non-limiting list of RNA-based hybridization methods which can be used to detect the miRNA of the present invention.

Northern Blot analysis—This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA, RNA or oligonucleotide (composed of deoxyribo or ribonucleotides) probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RNA in situ hybridization stain—In this method DNA, RNA or oligonucleotide (composed of deoxyribo or ribonucleotides) probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

Hybridization to oligonucleotide arrays—The chip/array technology has already been applied with success in numerous cases. For example, the screening of mutations has been undertaken in the BRCA1 gene, in S. cerevisiae mutant strains, and in the protease gene of HIV-1 virus [see Hacia et al., (1996) Nat Genet. 1996; 14(4):441-447; Shoemaker et al., (1996) Nat Genet. 1996; 14(4):450-456; Kozal et al., (1996) Nat Med 1996; 2(7):753-759].

The nucleic acid sample which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Probes that perfectly match a sequence of the nucleic acid sample generally produce stronger signals than those that have mismatches. Since the sequence and position of each probe immobilized on the chip is known, the identity of the nucleic acid hybridized to a given probe can be determined.

For single-nucleotide polymorphism analyses, sets of four oligonucleotide probes (one for each base type), preferably sets of two oligonucleotide probes (one for each base type of the biallelic marker) are generally designed that span each position of a portion of the candidate region found in the nucleic acid sample, differing only in the identity of the polymorphic base. The relative intensity of hybridization to each series of probes at a particular location allows the identification of the base corresponding to the polymorphic base of the probe.

It will be appreciated that the use of direct electric field control improves the determination of single base mutations (Nanogen). A positive field increases the transport rate of negatively charged nucleic acids and results in a 10-fold increase of the hybridization rates. Using this technique, single base pair mismatches are detected in less than 15 sec [see Sosnowski et al., (1997) Proc Natl Acad Sci USA 1997; 94(4):1119-1123].

Preferably, the oligonucleotide probes utilized by the various hybridization techniques described hereinabove are capable of hybridizing to the miRNA of the present invention (e.g., a polynucleotide having a nucleic acid sequence as set forth by SEQ ID NO:21 and/or 22) under stringent hybridization conditions.

By way of example, hybridization of short nucleic acids (below 200 bp in length, e.g. 17-40 bp in length) can be effected by the following hybridization protocols depending on the desired stringency; (i) hybridization solution of 6×SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACl, 0.01 M sodium phosphate (pH 6.8), 1 mM EDTA (pH 7.6), 0.5% SDS at 1-1.5° C. below the Tm (stringent hybridization conditions) (ii) hybridization solution of 6×SSC and 0.1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACl, 0.01 M sodium phosphate (pH 6.8), 1 mM EDTA (pH 7.6), 0.5% SDS at 1-1.5° C. below the Tm, final wash solution of 6×SSC, and final wash at 22° C. (stringent to moderate hybridization conditions); and (iii) hybridization solution of 6×SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3° C. below the Tm and final wash solution of 6×SSC at 22° C. (moderate hybridization solution).

For example, a micro-RNA molecule having a nucleic acid sequence as set forth in SEQ ID NO:21 can be detected using an oligonucleotide probe having a nucleic acid sequence as set forth in SEQ ID NO:2. It will be appreciated that detection of reduced levels of such micro-RNA in a bone marrow sample can be indicative of increased megakaryocyte differentiation in the subject from which the sample is obtained. On the other hand, detection of increased levels of such a micro-RNA can be indicative of decreased megakaryocyte differentiation and can be associated with several disorders such as thrombocytopenia, idiopathic thrombocytopenic purpura (ITP), congenital amegakaryocytic thrombocytopenia (CAMT), essential thrombocythemia (ET), and acquired amegakaryocytic thrombocytopenia (AATP).

As is mentioned before, the miRNA of the present invention can be also detected using a reverse-transcription based method. Reverse-transcription utilizes RNA template, primers (specific or random), reverse transcriptase (e.g., MMLV-RT) and deoxyribonucleotides to form (i.e., synthesize) a complementary DNA (cDNA) molecule based on the RNA template sequence. Once synthesized, the single strand cDNA molecule or the double strand cDNA molecule (which is synthesized based on the single strand cDNA) can be used in various DNA based detection methods.

Following is a non-limiting list of methods which can directly or indirectly be used to detect the micro-RNA of the present invention.

RT-PCR analysis—This method uses PCR amplification of relatively rare RNA molecules. First, RNA molecules are purified from cells and converted into complementary DNA (cDNA) using a reverse transcriptase enzyme (such as an MMLV-RT) and primers such as oligo-dT, random hexamers, or gene-specific primers. Then by applying gene-specific primers and Taq DNA polymerase, a PCR amplification reaction is carried out in a PCR machine. Those of ordinary skill in the art are capable of selecting the length and sequence of the gene-specific primers and the PCR conditions (i.e., annealing temperatures, number of cycles, and the like) that are suitable for detecting specific RNA molecules. It will be appreciated that a semi-quantitative RT-PCR reaction can be employed, by adjusting the number of PCR cycles and comparing the amplification product to known controls.

In situ RT-PCR stain—This method is described by: Nuovo, G. J. et al. (1993). Intracellular localization of polymerase chain reaction (PCR)-amplified hepatitis C cDNA. Am J Surg Pathol 17, 683-690); and Komminoth, P. et al. (1994) Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract 190, 1017-1025). Briefly, the RT-PCR reaction on fixed cells involves the incorporation of labeled nucleotides in the reaction. The reaction is effected using a specific in situ RT-PCR apparatus, such as the laser-capture microdissection PixCell II™ Laser Capture Microdissection (LCM) system available from Arcturus Engineering (Mountainview, Calif., USA).

Integrated systems—Another technique which may be used to analyze sequence alterations includes multicomponent integrated systems, which miniaturize and compartmentalize processes such as PCR and capillary electrophoresis reactions in a single functional device. An example of such a technique is disclosed in U.S. Pat. No. 5,589,136, which describes the integration of PCR amplification and capillary electrophoresis in chips.

Integrated systems are preferably employed along with microfluidic systems. These systems comprise a pattern of microchannels designed onto a glass, silicon, quartz, or plastic wafer included on a microchip. The movements of the samples are controlled by electric, electro-osmotic, or hydrostatic forces applied across different areas of the microchip, to create functional microscopic valves and pumps with no moving parts. Varying the voltage controls the liquid flow at intersections between the micro-machined channels and changes the liquid flow rate for pumping across different sections of the microchip.

When identifying sequence alterations, a microfluidic system may integrate nucleic acid amplification, microsequencing, capillary electrophoresis, and a detection method such as laser-induced fluorescence detection. In a first step, the DNA sample is amplified, preferably by PCR. The amplification product is then subjected to automated microsequencing reactions using ddNTPs (with specific fluorescence for each ddNTP) and the appropriate oligonucleotide microsequencing primers, which hybridize just upstream of the targeted polymorphic base. Once the extension at the 3′ end is completed, the primers are separated from the unincorporated fluorescent ddNTPs by capillary electrophoresis. The separation medium used in capillary electrophoresis can for example be polyacrylamide, polyethylene glycol, or dextran. The incorporated ddNTPs in the single-nucleotide primer extension products are identified by fluorescence detection. This microchip can be used to process 96 to 384 samples in parallel. It can use the typical four-color laser-induced fluorescence detection of ddNTPs.

It will be appreciated that when utilized along with automated equipment, the above-described detection methods can be both rapidly and easily used to screen multiple samples for the micro-RNA of the present invention.

Ligase Chain Reaction (LCR or LAR)—The ligase chain reaction [LCR; sometimes referred to as “Ligase Amplification Reaction” (LAR)] described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes. Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA)—The self-sustained sequence replication reaction (3SR) (Guatelli et al., Proc. Natl. Acad. Sci., 87:1874-1878, 1990), with an erratum at Proc. Natl. Acad. Sci., 87:7797, 1990) is a transcription-based in vitro amplification system (Kwok et al., Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase—In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)n=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.8520, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999, 1990)

A similar 3′-mismatch strategy is used with greater effect to prevent ligation in the LCR (Barany, PCR Meth. Applic., 1:5, 1991). Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR)—The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA—Branched DNA (bDNA), described by Urdea et al., Gene 61:253-264 (1987), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

Allele-specific oligonucleotides (ASOs)—In this method, an allele-specific oligonucleotide (ASO) is designed to hybridize in proximity to the polymorphic nucleotide, such that a primer extension or ligation event can be used as the indicator of a match or a mismatch. Hybridization with radioactively labeled ASOs has also been applied to the detection of specific SNPs (Connor, B. J. et al. (1983), Proc Natl Acad Sci USA, 80, 278-282). The method is based on the differences in the melting temperatures of short DNA fragments differing by a single nucleotide. Stringent hybridization and washing conditions can differentiate between mutant and wild-type alleles.

Denaturing/Temperature Gradient Gel Electrophoresis (DGGE/TGGE)—Two other methods rely on detecting changes in electrophoretic mobility in response to minor sequence changes. One of these methods, termed “Denaturing Gradient Gel Electrophoresis” (DGGE), is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can be used to detect the presence of SNPs in the target sequences due to the corresponding change in electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams, E. S. et al. (1990). Comprehensive detection of single base changes in human genomic DNA using denaturing gradient gel electrophoresis and a GC clamp. Genomics 7, 463-475). Attaching a GC clamp, to one primer is critical to ensure that the amplified sequence has a low dissociation temperature (Sheffield, V. C. et al. (1989). Attachment of a 40-Base-Pair G+C-Rich Sequence (GC-Clamp) to Genomic DNA Fragments by the Polymerase Chain Reaction Results in Improved Detection of Single-Base Changes. Proc Natl Acad Sci 86, 232-236; and Lerman, L. S. and Silverstein, K. (1987). Computational simulation of DNA melting and its application to denaturing gradient gel electrophoresis. Meth Enzymol 155, 482-501). Modifications of the technique have been developed using temperature gradients (Wartell, R. M. et al. (1990). Detecting base pair substitutions in DNA fragments by temperature-gradient gel electrophoresis. Nucl Acids Res, 18(9), 2699-2705) and the method can be also applied to RNA:RNA duplexes (Smith, F. I. et al. (1988). Novel method of detecting single base substitutions in RNA molecules by differential melting behavior in solution. Genomics 3(3), 217-223).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of the method called “Constant Denaturant Gel Electrophoresis” (CDGE) (Borresen, A. et al. (1991). Constant Denaturant Gel Electrophoresis as a Rapid Screening Technique for p53 Mutations. Proc Natl Acad Sci USA 88(19), 8405-8409). CDGE requires that gels be run under different denaturant conditions in order to reach high efficiency for the detection of SNPs.

A technique analogueous to DGGE, termed “Temperature Gradient Gel Electrophoresis” (TGGE), uses a thermal gradient rather than a chemical denaturant gradient (Scholz, R. B. et al. (1993). Rapid screening for Tp53 mutations by temperature gradient gel electrophoresis: a comparison with SSCP analysis. Hum Mol Genet. 2(12), 2155-2158). TGGE requires the use of specialized equipment that can generate a temperature gradient perpendicularly oriented relative to the electrical field. TGGE can detect mutations in relatively small fragments of DNA; therefore, scanning large gene segments requires the use of multiple PCR products prior to running the gel.

Single-Strand Conformation Polymorphism (SSCP)—Another common method, called “Single-Strand Conformation Polymorphism” (SSCP), was developed by Hayashi, Sekya, and colleagues (reviewed by Hayashi, K (1991). PCR-SSCP: A simple and sensitive method for detection of mutations in the genomic DNA. PCR Meth Appl 1, 34-38), and is based on the observation that single-strand nucleic acids can take on characteristic conformations under non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, M. et al. (1989a). Rapid and sensitive detection of point mutations and DNA polymorphisms using the polymerase chain reaction. Genomics 5, 874-879; Orita, M. et al. (1989b). Detection of Polymorphisms of Human DNA by Gel Electrophoresis as Single-Strand Conformation Polymorphisms. Proc Natl Acad Sci USA 86, 2766-2770).

The SSCP process involves denaturing a DNA segment (e.g., a PCR product) that is labeled on both strands, followed by slow electrophoretic separation in a non-denaturing polyacrylamide gel to allow intra-molecular interactions to form without disturbance during the run. This technique is extremely sensitive to variations in gel composition and temperature. A serious limitation of this method is the relative difficulty encountered in comparing data generated in different laboratories, under apparently similar conditions.

Dideoxy fingerprinting (ddF)—Dideoxy fingerprinting (ddF) is another technique developed to scan genes for the presence of mutations (Liu, Q. and Sommer, S. S. (1994). Parameters affecting the sensitivities of dideoxy fingerprinting and SSCP. PCR Methods Appl 4, 97-108). The ddF technique combines components of Sanger dideoxy sequencing with SSCP. First, a dideoxy sequencing reaction is performed using one dideoxy terminator. Next, the reaction products are electrophoresed on non-denaturing polyacrylamide gels to detect alterations in mobility of the termination segments, as in SSCP analysis. While ddF is an improvement over SSCP in terms of increased sensitivity, ddF requires the use of expensive dideoxynucleotides and the technique is still limited to the analysis of fragments of the size suitable for SSCP (i.e., fragments of 200-300 bases) for optimal detection of mutations.

In addition to the above limitations, all of these methods for detecting single mutations are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have especially severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base-pair fragments. Similarly, the sensitivity of DGGE decreases as the length of the fragment reaches 500 base pairs. The ddF technique, as a combination of direct sequencing and SSCP, is also limited by the relatively small size of the DNA that can be screened.

Pyrosequencing™ analysis—This technique (Pyrosequencing, Inc., Westborough, Mass., USA) is based on the hybridization of a sequencing primer to a single-stranded, PCR-amplified DNA template in the presence of DNA polymerase, ATP sulfurylase, luciferase, and apyrase enzymes and the adenosine 5′-phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′-phosphosulfate. The ATP drives the luciferase-mediated conversion of luciferin to oxyluciferin that generates visible light in amounts that are proportional to the amount of ATP. The light produced in the luciferase-catalyzed reaction is detected by a charge-coupled device (CCD) camera and seen as a peak in a pyrogram™. The strength of each light signal is proportional to the number of nucleotides incorporated.

Acycloprime™ analysis—This technique (PerkinElmer, Boston, Mass., USA) is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat-inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the domain Archaea, and a pair of AcycloTerminators™ labeled with R110 and TAMRA, representing the possible alleles for the SNP of interest. AcycloTerminator non-nucleotide analogues are biologically active with a variety of DNA polymerases. Similarly to 2′,3′-dideoxynucleotide-5′-triphosphates, the acyclic analogues function as chain terminators. The analogue is incorporated by the DNA polymerase in a base-specific manner onto the 3′-end of the DNA chain; since there is no 3′-hydroxyl, the polymerase is unable to function in further chain elongation. It has been found that AcycloPol has a higher affinity and specificity for derivatized AcycloTerminators than various Taq mutants have for derivatized 2′,3′-dideoxynucleotide terminators.

Reverse dot-blot—This technique uses labeled sequence-specific oligonucleotide probes and unlabeled nucleic acid samples. Activated primary amine-conjugated oligonucleotides are covalently attached to carboxylated nylon membranes. After hybridization and washing, the labeled probe or a labeled fragment of the probe can be released using oligomer restriction, i.e., the digestion of the duplex hybrid with a restriction enzyme. Circular spots or lines are visualized colorimetrically after incubation with streptavidin horseradish peroxidase, followed by development using tetramethylbenzidine and hydrogen peroxide, or alternatively via chemiluminescence after incubation with avidin alkaline phosphatase conjugate and a luminous substrate susceptible to enzyme activation, such as CSPD, followed by exposure to x-ray film.

It will be appreciated that advances in the field of SNP detection have provided additional accurate, easy, and inexpensive large-scale SNP genotyping techniques, such as: dynamic allele-specific hybridization (DASH) (Howell, W. M. et al. (1999). Dynamic allele-specific hybridization (DASH). Nat Biotechnol 17, 87-88); microplate array diagonal gel electrophoresis (MADGE) (Day, I. N. et al. (1995). High-throughput genotyping using horizontal polyacrylamide gels with wells arranged for microplate array diagonal gel electrophoresis (MADGE). Biotechniques 19, 830-835); the TaqMan® system (Holland, P. M. et al. (1991). Detection of specific polymerase chain reaction product by utilizing the 5′→3′ exonuclease activity of Thermus aquaticus DNA polymerase. Proc Natl Acad Sci USA 88, 7276-7280); various DNA “chip” technologies such as GeneChip® microarrays (e.g., SNP chips, Affymetrix, USA), which is disclosed in U.S. Pat. No. 6,300,063 to Lipshutz et al. 2001, which is fully incorporated herein by reference; genetic bit analysis (GBA®), described by Goelet, P. et al. (PCT Appl. No. 92/15712); peptide nucleic acids (PNA) (Ren, B. et al. (2004). Straightforward detection of SNPs in double-stranded DNA by using exonuclease III/nuclease S1/PNA system. Nucleic Acids Res. 32(4), e42) and locked nucleic acid (LNA) probes (Latorra, D. et al. (2003). Enhanced allele-specific PCR discrimination in SNP genotyping using 3′ locked nucleic acid (LNA) primers. Hum Mutat 22(1), 79-85); molecular beacons (Abravaya, K. et al. (2003). Molecular beacons as diagnostic tools: technology and applications. Clin Chem Lab Med 41, 468-474); intercolating dyes (Germer, S, and Higuchi, R. (1999). Single-tube genotyping without oligonucleotide probes. Genome Res 9, 72-78); FRET primers (Solinas, A. et al. (2001). Duplex Scorpion primers in SNP analysis and FRET applications. Nucleic Acids Res 29(20), E96); AlphaScreen™ (Beaudet, L. et al. (2001). Homogeneous assays for single-nucleotide polymorphism typing using AlphaScreen. Genome Res 11(4), 600-608); SNPstream® (Bell, P. A. et al. (2002). SNPstream UHT: ultra-high throughput SNP genotyping for pharmacogenomics and drug discovery. Biotechniques Supplement 70-72, 74, 76-77); multiplex minisequencing (Curcio, M. et al. (2002). Multiplex high-throughput solid-phase minisequencing by capillary electrophoresis and liquid core waveguide fluorescence detection. Electrophoresis 23(10), 1467-1472); SnaPshot™ Multiplex System (Turner, D. et al. (2002). Typing of multiple single nucleotide polymorphisms in cytokine and receptor genes using SNaPshot. Hum Immunol 63(6), 508-513); MassEXTEND™ (Cashman, J. R. et al. (2001). Population distribution of human flavin-containing monooxygenase form 3: gene polymorphisms. Drug Metab Dispos 29, 1629-1637); GOOD assay (Sauer, S, and Gut, I. G. (2003). Extension of the GOOD assay for genotyping single nucleotide polymorphisms by matrix-assisted laser desorption/ionization mass spectrometry. Rapid Commun Mass Spectrom 17, 1265-1272); microarray minisequencing (Liljedahl, U. et al. (2003). A microarray minisequencing system for pharmacogenetic profiling of antihypertensive drug response. Pharmacogenetics 13, 7-17); arrayed primer extension (APEX) (Tonisson, N. et al. (2000). Unravelling genetic data by arrayed primer extension. Clin Chem Lab Med 38, 165-170); microarray primer extension (O'Meara, D. et al. (2002). SNP typing by apyrase-mediated allele-specific primer extension on DNA microarrays. Nucleic Acids Res 30, e75); tag arrays (Fan, J. B. et al. (2000). Parallel genotyping of human SNPs using generic high-density oligonucleotide tag arrays. Genome Res 10(6), 853-860); template-directed incorporation (TDI) (Akula, N. et al. (2002). Utility and accuracy of template-directed dye-terminator incorporation with fluorescence-polarization detection for genotyping single nucleotide polymorphisms. Biotechniques 32, 1072-1076, 1078); fluorescence polarization (Hsu, T. M. et al. (2001). Universal SNP genotyping assay with fluorescence polarization detection. Biotechniques 31, 560, 562, 564-568, passim); colorimetric oligonucleotide ligation assay (OLA) (Nickerson, D. A. et al. (1990). Automated DNA diagnostics using an ELISA-based oligonucleotide ligation assay. Proc Natl Acad Sci USA 87, 8923-8927); sequence-coded OLA (Gasparini, P. et al. (1999). Analysis of 31 CFTR mutations by polymerase chain reaction/oligonucleotide ligation assay in a pilot screening of 4476 newborns for cystic fibrosis. J Med Screen 6, 67-69); microarray ligation; ligase chain reaction; padlock probes; rolling circle amplification; invader assays (Shi, M. M. (2001). Enabling large-scale pharmacogenetic studies by high-throughput mutation detection and genotyping technologies. Clin Chem 47, 164-172); coded microspheres (Rao, K. V. et al. (2003). Genotyping single nucleotide polymorphisms directly from genomic DNA by invasive cleavage reaction on microspheres. Nucleic Acids Res 31, e66); MassARRAY™ (Leushner, J. and Chiu, N. H. (2000). Automated mass spectrometry: a revolutionary technology for clinical diagnostics. Mol Diagn 5, 341-348); heteroduplex analysis; mismatch cleavage detection; exonuclease-resistant nucleotide derivative (U.S. Pat. No. 4,656,127); and other conventional techniques as described in: Sheffield et al. (1989); White, M. B. et al. (1992). Detecting single base substitution as heteroduplex polymorphisms. Genomics 12, 301-306; Grompe, M. et al. (1989). Scanning detection of mutations in human ornithine transcarbamoylase by chemical mismatch cleavage. Proc Natl Acad Sci USA 86(15), 5888-5892; and Grompe, M. (1993). The rapid detection of unknown mutations in nucleic acids. Nat Genet. 5, 111-117.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which, together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth by U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D. and Higgins S. J., Eds. (1985); “Transcription and Translation” Hanes, B. D. and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

If micro-RNA-regulated signaling is intimately involved in the proliferation of leukemic cells, the suppression of such proliferation should be reflected in changes in the levels of cholinergic proteins, such as AChE-R, which terminate cholinergic signals. Reciprocally, the suppression of ACHE-R should be reflected in changed levels of the specific signaling proteins that are activated during hematopoietic proliferation. However, the cellular reactions executing such cholinergic signals are unknown. To examine the micro-RNA-associated cholinergic signaling pathway, the present inventors used the process of megakaryocytopoiesis, the maturation of platelet-forming megakaryocytes (MKs) which involves cholinergic modulation (Patinkin et al., 1990; Soreq et al., 1994; Pick M, et al., 2004, Annals of New York Academy of Science, 1018: 85-95) as a cellular model. In order to evaluate the effect of cholinergic signaling on cell proliferation and cell death, specific inhibitors of enzymes involved in cholinergic signal cascades were applied to cultures of Meg-01 cells. In addition, the effects of mitochondrial function and Ca²⁺ release on the natural and AChE-R-induced proliferation of leukemic cell lines (e.g., the megakaryocytic line, Meg-01) were determined, as follows.

### General Materials and Experimental Methods

Chemicals—Bisindolylmaleimide (BIM; PKC inhibitor), N-(2-((p-Bromocinnamyl)amino) ethyl)-5-isoquinolinesulfonamide (H89; PKA inhibitor) were purchased from Calbiochem (San Diego, Calif., USA); 1,5-Bis(4-allyldimethylammoniumphenyl)pentan-3-one dibromide (BW 284c51-BW; AChE inhibitor), Physostigmine (Eserine; AChE inhibitor), Pyridostigmine (ACHE inhibitor) and Thapsigargin (Ca²⁺-ATPase inhibitor), Actinomycin D (transcription inhibitor), Bongkrekic acid (inhibitor of the adenine nucleotide translocator), Etoposide (chemotherapy drug), beta-Mercaptoethanol, Carbachol (cholinergic agonist), E. coli lipopolysaccharide (LPS), diethyl-p-nitrophenyl phosphate (paraoxon) and 1-Methyl-4-phenyl-1,2,3,6-tetrahydropyridine (MPTP) were all purchased from Sigma.

Peptides—The human AChE-R C-terminal peptide ARP (GMQGPAGSGWEEGSGSPPGVTPLFSP; SEQ ID NO:3) was purchased from the American Peptide Company (Sunnyvale, Calif., USA). The AChE-S C-terminal peptide ASP (DTLDEAERQWKAEFHRWSSYMVHWKNQFDHYSKQDRCSDL; SEQ ID NO:4) was prepared as detailed elsewhere (Grisaru et al., 2001).

Cell cultures—MEG-01 cells, gratefully received from Dr. V. Deutsch (Tel Aviv, Israel) were cultured in Iscove's Minimal Dulbecco's Medium (IMDM) (Gibco-BRL), containing 10% heat-inactivated donor horse serum (DHS) in a fully humidified atmosphere at 37° C. in 5% CO₂. Half the media was replaced every 3 days. Cells were plated in a density of 1×10⁶ cells/ml in 6-well Nunclon™ plates (Nalge Nunc International, Denmark) and were incubated with the noted agents for 18 hours. For immunohistochemistry and in situ hybridization analyses, cells were fixed for 1 hour with freshly prepared 4% paraformadehyde in phosphate buffered saline (PBS—phosphate buffer 0.1 M at pH 7.4 and 0.9% NaCl), washed, resuspended in PBS and kept at 4° C. Prior to immunocytochemistry 25 μl samples of cell suspension were applied to 18-mm coverslips coated with poly-L-ornithine and allowed to dry at room temperature.

RAW 264.7 macrophage cell line; Abelson murine leukemia virus transformed was obtained from American Type Culture Collection (ATCC, Manassas, Va.) and cultured at a concentration of 1×10⁶ cells/ml in Iscove's Minimal Dulbecco's Medium (IMDM) (GIBCO-BRL), with 10% heat-inactivated horse serum (37° C., 5% CO₂, 50% replaced every 3 days) medium.

Antisense oligonucleotides—Antisense oligodeoxynucleotides (AS) were used to selectively suppress ACHE-R mRNA levels in Meg-01 cells. EN101, a 20-mer anti-AChE mRNA 2-O-methyl-AS-ODN antisense (SEQ ID NO:5-5′-CTGCGATATTTTCTTGTACC-3′), was designed to target exon 2, a common exon to both AChE-S and ACHE-R transcripts of mammalian AChE. EN101 was previously found to preferably induce destruction of nascent AChEmRNA transcripts (Perry et al., 2004). The oligonucleotide was protected against nuclease degradation by capping the three 3-terminal nucleotides by 2-O-methyl groups as described in Galyam et al., 2001.

DNA containing unmethyatde CpG motives—ODN 2006 is a 24-mer oligonucleotide [5′-TCGTCGTTTTGTCGTTTTGTCGTT-3′ (SEQ ID NO:19)] (Hartmann, G, et al., 1999, PNAS, 96: 9305-9310). CpG ODN2216 is a 20-mer oligonucleotide [5′-GGGGGACGATCGTCGGGGGG-3′ (SEQ ID NO:12)] (Domeika K, et al., 2004, Vet Immunol Immunopathol. 101: 87-102). These DNA containing unmethylated CpG motifs serve as potent stimuli for inducing dendritic cell survival, activation, maturation and ability to promote T helper 1 (Th1)-like T cell response. ODN 2006 enhanced expression of CD54 and CD40 while its methyated version (ODN 2117) failed to enhance such expression [Hartmann, 1999 (Supra)].

Induction of cellular stress response and caspase activation pathway—Induction of cellular stress response and caspase activation pathway was initiated by incubating the cells with 10 nM thapsigargin, a known modifier of cell fate decisions and an inhibitor of ER Ca²⁺ pumps, which resulted in the release of intracellular calcium stores. Thapsigargin-treated cells were incubated with actinomycin D (2 μg/ml), H89 (10 μM) and BIM (10 μM) as indicated. To test the causal relationship of ACHE induction and caspase-3 activation with thapsigargin, the cells were incubated with physostigmine (10 μM), pyridostigmine (1 μM), or BW284c51 (10 μM), all small-molecule inhibitors of AChE, or EN101 (3 nM; SEQ ID NO:5), an antisense oligonucleotide suppressor of ACHE-R mRNA translation.

Induction of cellular stress responses and caspase-3 activation was also effected using ARP (SEQ ID NO:3), the ACHE Read-through Peptide (2 nM, unless otherwise indicated), which is modeled on the C-terminus of ACHE-R. Bongkrekic acid, an inhibitor of the adenine nucleotide translocator, which is one of the components of the permeability transition pore (PTP), was used in the concentrations indicated to test whether mitochondria participate in caspase-3 activation induced by ARP.

Induction of caspase-associated apoptosis pathway—To induce the apoptosis pathway in Meg-01 cells, cells were incubated for 24 hours with the chemotherapy drug Etoposide (50 μM) or beta-Mercaptoethanol (20 mM). Carbachol (2 μM), a cholinergic agonist, was used to test whether ACHE-S expression correlates with cell death.

Immunocytochemistry staining—Immunohistochemistry staining was performed using antibodies against activated caspase-3 (polyclonal, Cell Signaling Technology Inc., Beverly, Mass., USA) at a 1:200 dilution; ACHE-S C16 at a dilution of 1:100, AChE N19 (targeted against the N-terminal 19 amino acid residues of human AChE) at a dilution of 1:20 and antibody against Bcl-2 (which inhibits caspase-3 activation and apoptosis) at a dilution of 1:100 (Santa Cruz Biotechnology Inc., Santa Cruz, Calif., USA); Anti-ARP (AChE-R) (Sternfeld et al., 2000) at a dilution of 1:50; anti-c-Myc a dilution of 1:100 (Santa Cruz Biotechnology Inc., Santa Cruz, Calif., USA); antibody against SC35 (a splicing factor used as a marker for the presence of pre-mRNA splicing machinery) was used at a dilution of 1:50 (Pharmingen, BD Biosciences, Becton-Dickinson, Oxford, UK); antibody against GATA-1 H200 (a transcription factor known to participate in the differentiation of megakaryocytes) was used at a dilution of 1:100 (Santa Cruz Biotechnology Inc., Santa Cruz, Calif., USA); antibody against PKCbetaII was used at a dilution of 1:50 (Santa Cruz Biotechnology Inc., Santa Cruz, Calif., USA). Briefly, paraformaldehyde-fixed cells were incubated for 30 minutes in 3% H₂O₂ in PBS, washed in PBS and blocked for 1 hour at room temperature with a blocking buffer (5% BSA, 0.8% Triton X-100 in PBS) and further incubated overnight at 4° C. with the noted primary antibodies. Detection was performed using the HRP-ABC kit (Vectastain, Vector Labs, Burlingame, Calif., USA). Cells were coverslipped in Shandon immunomount and analyzed by light or fluorescence microscopy using a Zeiss Axiophot microscope equipped with a digital camera. Quantitative image analysis of the fluorescent signals was with the software package ImagePro4. Using an n=at least 100 cells, fluorescence of individual cells was measured and the results were classified in 14 levels.

In situ hybridization—In situ hybridization was performed essentially as previously described (Galyam et al., 2001). Briefly, Meg-01 cells were concentrated by centrifugation at 4° C. for 5 minutes at 2000 rpm, fixed for 30 minutes in 4% paraformaldehyde in PBS, washed twice with PBT (PBS with 0.1% Tween-20), incubated for 10 minutes with 100 mM glycine in PBS and washed in PBT. Prehybridization was performed for 1 hour at 65° C. in the presence of an hybridization buffer containing 50% formamide, 750 mmol/l sodium chloride, 75 mmol/l sodium citrate at pH 4.5, 50 μg/ml heparin and 50 μg/mL tRNA. Hybridization was performed for 90 minutes at 52° C. in the presence of 1 μg/ml digoxigenin (DIG; Boehringer)-labeled probe specific to the human ACHE-R (5′-CCGGGGGACGUCGGGGUGGGGUGGGGAUGGGCAGAGUCUGGGGCUCGU CU-3′; SEQ ID NO:10). The 5′-biotinylated, 2-O-methylated AChE cRNA probe (5′-CCGGGGGACGUCGGGGUGGGGUGGGGAUGGGCAGAGUCUGGGGCUC GUCU-3′; SEQ ID NO:11) complementary to human AChE-R mRNA was purchased from Microsynth GMBH (Balgach, Switzerland). Hybridization signal was analysed using a fluorescence microscope (Zeiss Axiophot) equipped with a digital camera. Quantitative image analysis of the fluorescent signals resulting from AChE-R mRNA staining was performed using the software package ImagePro4. Using an n=at least 100 cells, fluorescence of individual cells was measured and the results were classified in 14 levels.

Quantifying microRNA levels in Meg-01 cells—RNA was extracted from Meg01 cells using the RNeasy kit (Beit Haemek, Israel) according to manufacturer's instructions. RNA concentration was verified using a spectrophotometer. Reverse transcription was carried out using the Promega RT kit and gene-specific 3′ primers for the human miRNA-181a precursor (SEQ ID NO:6; 5′-GGTACAGTCAACGGTCAGTGG-3′) or the actin RNA (5′-TGAAACAACATACAATTCCATCATGAAGTGTGAC-3′; SEQ ID NO:8 and 5′-5′-AGGAGCGATAATCTTGATCTTCATGGTGCT-3′; SEQ ID NO:9.

Quantitative real-time PCR was performed using the Roche LightCycler and the Roche FastStart DNA amplification kit. PCR conditions included annealing temperature of 64° C. and amplification using the following primer pairs: for huma miRNA-181a precursor the forward and reverse primers were 5′-GGACTCCAAGGAACATTCAACG-3′ (SEQ ID NO:7) and 5′-GGTACAGTCAACGGTCAGTGG-3′ (SEQ ID NO:6), respectively; for the human actin RNA the forward and reverse primers were SEQ ID NO:9 (forward primer) and SEQ ID NO:8 (reverse primer), respectively. The primers were designed using the Sequence Analysis software for Mac OS X and were purchased from Sigma Biochemicals. Presence of amplified pre-miRNA-181a was verified by cloning and sequencing of the PCR product. The resulting amplification data was analyzed using OpenOffice 1.1 software for Mac OS X. The data obtained for human actin was used for normalization.

Electron Microscopy—Transmission electron microscopy and scanning electron microscopy were used to monitor Meg-01 cells undergoing apoptosis and differentiation for morphological changes.

Cell cycle analysis—DNA content was determined by propidium iodide (PI) staining of fixed cells followed by flow cytometry. Cells were washed twice in phosphate buffer saline (PBS), fixed overnight in 100% ethanol at 4° C., washed twice in 0.5% bovine serum albumin (BSA) in PBS, resuspended in 1 ml of staining solution (PBS containing 0.05 mg/mL PI, and 1 mg/mL RNAse), and incubated for 30 minutes at 37° C. DNA content was analyzed in a FACScalibur flow cytometer (Becton-Dickinson, Oxford, UK) and cell cycle distribution analyses were performed using Cellquest software (Becton-Dickinson, Oxford, UK).

Ploidy analysis by FACS—For cell proliferation assay, cells were incubated for 6 hours with 5′ bromo-2-deoxyuridine (BrdU) and the cell polidy was assessed 30 hours post-treatment essentially as described in Grisaru et al., 1999. Fluorescence-activated cell sorting (FACS) was used to determine the ploidy of Meg-01 cells at 24, 48, and 72 hours post-treatment with ARP or thapsigargin. Phorbol myristate acetate (PMA, 10 μM) was used as a positive control. The cells were observed by photography for high-resolution detection, localization, and quantification of time-dependent changes in mRNA and protein variants and of subtle morphological changes. This was accomplished using the 6-parameter, 4-color flow cytometry, performed on a FACS™ Calibur (BD Bioscience), of at least 50,000 events per sample. Fluorescent detector sensitivity was set and monitored with Quantum™ beads (Bangs Laboratories). Data analysis was performed using CellQuest™ and CellQuest™ Pro software (Becton Dickinson).

Blood cell proliferation—BrdU incorporation was measured as detailed in Perry et al., 2004. Briefly cell counts were determined on a Zeiss Axiophot microscope, using a magnification of ×400. The results were expressed as the average±S.E.M. of the percentage of positive cells in four independent fields in the same coverslip (n=at least 100 cells/field).

DNA fragmentation analysis by the TUNEL assay—Oligonucleosomal fragmentation of DNA, the hallmark of cell death by apoptosis, was detected in situ using the Terminal deoxynucleotidyl transferase-mediated UTP Nick-End Labeling (TUNEL) assay according to manufacturer's instructions (DeadEnd kit from Promega).

Adhesion assay—Adhesion assays were performed as described elsewhere (Genever et al., 1999). Briefly, MEG-01 cells (2×10⁵ cells/ml) were cultured for 72 hours in 96-well plates, following which nonadherent cells were removed by three washes of PBS. Adherent cells were fixed in 70% ethanol (15 minutes) and stained with 0.5% crystal violet (25 minutes), followed by extensive washing with water to remove unbound dye. Dye was eluted by the addition of 50% ethanol/0.1 mol/l sodium citrate, pH 4.2. Absorbance was measured on a plate reader at 570 nm.

AChmiON—A fully 2′-O-methylated oligonucleotide (modified—SEQ ID NO:23; unmodified—SEQ ID NO:1) with the sequence of human/murine miRNA-181a (SEQ ID NO:21) was synthesized at Microsynth, Switzerland. The oligo was added to the medium of Meg01 cells or 293 HEK cells at a final concentration of 100 nM, and cells were maintained in normal culture conditions for 24 hours. A similar oligo (SEQ ID NO:20) with an inverse sequence (Microsynth) was used as a negative regulator of miRNA-181a.

Co-administration of thapsigargin and synthetic microRNAs to Meg-01 cells—Cultured Meg-01 cells were co-incubated with thapsigargin (10 nM) and synthetic AChmiON (miR-181) (SEQ ID NO:23; at 100 nM) or anti-miR-181 (SEQ ID NO:24 (modified, identical in sequence to SEQ ID NO:2); at 100 nM.

In vivo injections—FVB/N mice (control) and TgR mice (Sternfeld et al., J. Physiol. Paris, 1998) were injected intraperitoneally with low doses (50 μg/kg body weight) of the inflammatory stressor, E. coli lipopolysaccharide (LPS).

In addition, FVB/N mice were injected with sub-lethal doses of the anticholinesterase insecticide diethyl-p-nitrophenyl phosphate (paraoxonethyl (Sigma, Israel)) at 1 mg/kg body weight was injected twice at 0.5 mg/Kg doses 4 hours apart or the dopaminergic poison 1-Methyl-4-phenyl-1,2,3,6-tetrahydropyridine (MPTP) 60 mg/kg body weight at four injections of 15 mg/ml at 2 hour intervals. Mice were anesthetized and decapitated 72 hours following injections.

Determination of microRNA levels in bone marrow and intestine—Quantitative RT-PCR using primers for miRNA-181a (SEQ ID NOs:6 and 7) was employed to quantify micro-RNA levels in these tissues, with samples taken at 24, 48, and 72 hours post-injection.

NO₂ release was assayed according to Green (Green L C, et al., Anal Biochem 1982; 126(1):131-8. Briefly, equal volumes of Griess reagent (1% sulfanilamide/0.1% naphthylethylenediamine dihydrochloride/2.5% H₃PO₄) were incubated with supernatant samples (100 μl of medium in which cells had been cultured) for 10 minutes at room temperature and absorbance was measured at 546 nm in a micro-ELISA reader (TECAN). NO₂ concentration (in μM) was determined using NaNO₂ as a standard.

### Example 1

### Thapsagargin-Induced Megakaryocytic Differentiation Associates with PKC, PKA and AChE-Dependent Decreases in AChmiRNA

Prior studies have shown that the intracellular level of calcium is differentially regulated throughout megakaryocytes maturation of (den Dekker et al., 2001) and that this process involves the endoplasmic reticulum ER (Lacabaratz-Porret et al., 2000). The ER enters a profound reorganization during megakaryocytopoiesis, suggesting a pivotal role for calcium-regulated mechanisms during megakaryocyte maturation. The present inventors have hypothesized that calcium might induce megakaryocyte differentiation via a micro-RNA (miRNA) pathway.

Thapsigargin (Thapsi) is a sesquipentene lactone, a known modifier of cell fate decisions that discharges calcium into the intracellular milieu by inhibiting the Ca²⁺-ATPase of the endoplasmic reticulum (ER) (Thastrup et al., 1990). Thapsi can induce cell death (Chiarini et al., 2003) or inhibit it (Lotem et al., 2003), induce expression of activation-related molecules (Rodrigues Mascarenhas et al., 2003), inhibit or induce differentiation (Koski et al., 1999; Porter et al., 2002; Shi et al., 2000) and induce expression of immediate early genes (Studzinski et al., 1999).

Experimental Results

The level of AChmiRNA (miRNA-181a) is regulated by ER-calcium release—AChmiRNA (miRNA-181a; precursor molecule—SEQ ID NO:13; mature molecule—SEQ ID NO:1; FIGS. 2a and b) was shown to affect differentiation in the lymphocytic and myelocytic lineages (Chen et al., 2004; Kawashima et al., 2004) miRNA 181a also induces proliferation of the lung carcinoma cell line A549 (Cheng et al., (2005) Nuc. Acids Res. 33/4:1290-7). To test the hypothesis that calcium, miRNAs and cholinergic signaling might play inter-related roles in megakaryocytic differentiation, cells of the megakaryocytic line, Meg-01 (Lev-Lehman et al., 1997), were treated with Thapsi to induce ER-calcium release and the level of AChmiRNA was analyzed by real-time RT-PCR using the AChmiRNA specific primers SEQ ID NOs:6 and 7 (amplicon—SEQ ID NO:14). As is shown in FIG. 2c, ER-calcium release (Thapsi treatment) decreased the levels of AChmiRNA by 50%. However, co-treatment of Meg-01 cells with Thapsi and either H89 (the PKA inhibitor), BIM (the PKC inhibitor) or Physostigmine (the ACHE inhibitor) restored the control levels of AChmiRNA.

ER-calcium release induced Meg-01 differentiation—The effect of Thapsi upon megakaryocytes was uncovered using various techniques. To analyze the effect of calcium release on the cell surface of megakaryocytes, scanning electron microscopy was employed. Upon 24 hour of Thapsi treatment (FIGS. 3b and c), the Meg-01 cells exhibited a very early stage in the formation of demarcation membranes, appearing as irregular flat sheets on the cell surface (FIG. 3b). During the following phase, the entire cell surface was decorated with filopodia-like demarcation membranes, known to be a characteristic feature of early stages of megakaryocytic maturation (FIG. 3c). These results demonstrate that calcium release induces megakaryocyte maturation.

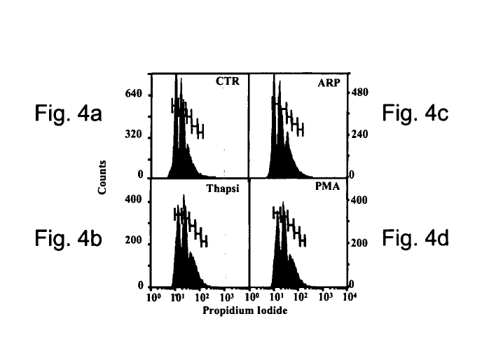

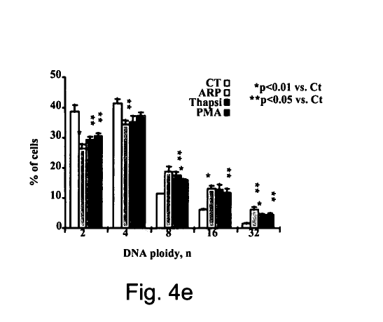

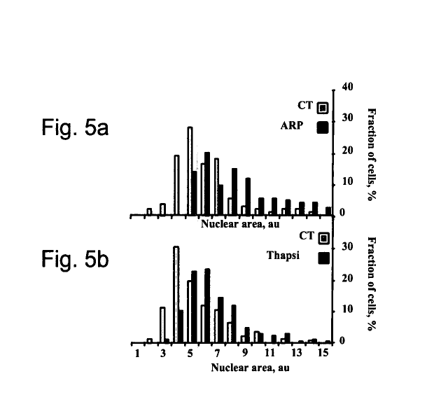

Thapsi treatment increased megakaryocyte polypolidy and nuclear area—Early megakaryocytic differentiation is followed by progressive polyploidization, acquisition of the lineage-specific markers and in the later stages of megakaryopoiesis differential expression of specific genes. Polyploidization is a unique feature of megakaryocytes in which repeated rounds of DNA replication occur without concomitant cell division, increasing DNA content progressively from 8N up to 128N (Ravid et al., 2002). To confirm that ER-calcium release induced megakaryocyte differentiation, the DNA content of Meg-01 cells was analyzed by Fluorescent Activated Cell Sorting (FACS) analysis and propidium iodide labeling. The DNA histograms of FIGS. 4a-d and 4e are representative of three independent FACS and propidium iodide incorporation experiments, respectively. Upon 72 hours of Thapsi treatment a bigger fraction of cells became polyploid (i.e., DNA content more than 4N), an effect very similar to what was observed upon treatment with PMA (phorbol 12-myristate 13-acetate), which was used as a positive control (Long et al., 1984) (FIGS. 4a-e). Thus, AChmiRNA downregulation (FIG. 2c) associates with an increase in the fraction of polyploid cells and an increase in the level of their ploidy. The hyperdiploid complement of DNA within a single nucleus also leads to an increase in its size. As is further shown in FIG. 5b, the ER calcium release was associated with an increase in the nuclear area, reinforcing the correlation between AChmiRNA downregulation and maturation and differentiation of Meg-01 cells.

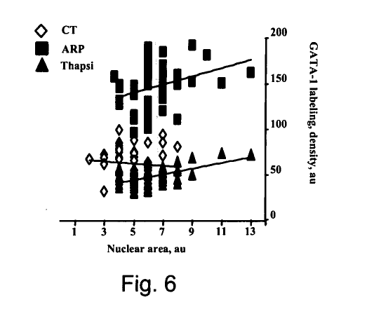

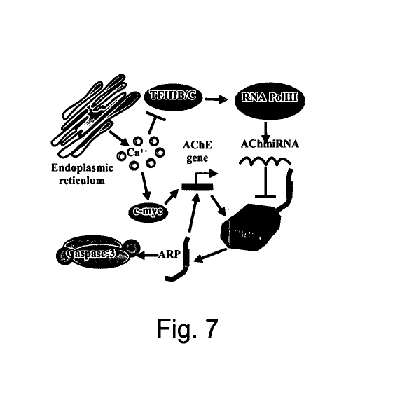

GATA-1 is a zinc finger transcription factor that is expressed in erythroid cells, megakaryocytes, mast cells and eosinophils (Weiss and Orkin, 1995). Functional GATA elements are present in the proximal promoters of virtually all erythroid- and megakaryocyte-restricted genes examined and it was demonstrated to be required for the normal maturation of both erythroid and megakaryocytic cells (Fujiwara et al., 1996; Pevny et al., 1991; Pevny et al., 1995; Shivdasani et al., 1997). The correlation between nuclei size and expression of GATA-1 in cells treated with Thapsi was examined. As is shown in FIG. 6, Thapsi-treated cells showed a correlation between the two parameters (R2=0.1992) that was not observed in the control cells (R2=0.0006), supporting the notion of Ca⁺⁺ involvement in GATA-1 expression.

ARP treatment induced megakryocyte maturation, polypolidy and increased nuclear area—ARP (SEQ ID NO:3), a peptide derived from C-terminal sequence of ACHE-R, was used as tool to further assess the role of ACHE. Similar to Thapsi treatment, ARP induced cell surface modifications (FIGS. 3d-f), increased ploidy (FIGS. 4a-e) and nuclei size (FIG. 5a) as well as positive GATA-1/nuclei area correlation (R2=0.1304) (FIG. 6).

Thus, Thapsi and ARP treatments resulted in similar cell differentiation profiles. These results suggest that the cholinergic signaling cascade induced by ARP (Pick et al., 2004) involves an intracellular Ca⁺⁺ release phase.

Altogether, these data show that downregulation of AChmiRNA triggered by ER-calcium release induced differentiation of megakaryoblasts; that the ACHE inhibitor Physostigmine prevented the decrease of AChmiRNA caused by calcium and that ARP, an AChE-R derived peptide, exerted the same effects as Thapsi on megakaryocytic differentiation.

### Example 2

### AChmiRNA Decrease is Associated with Splice Shift in AChE mRNA and Differentiation-Induced Caspase-3 Activation

Experimental Results

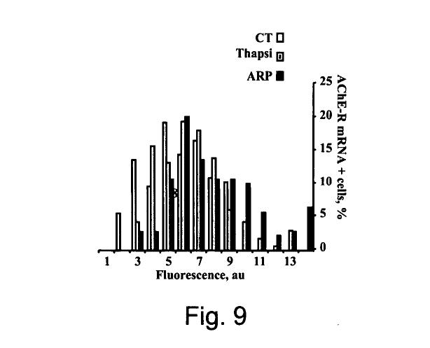

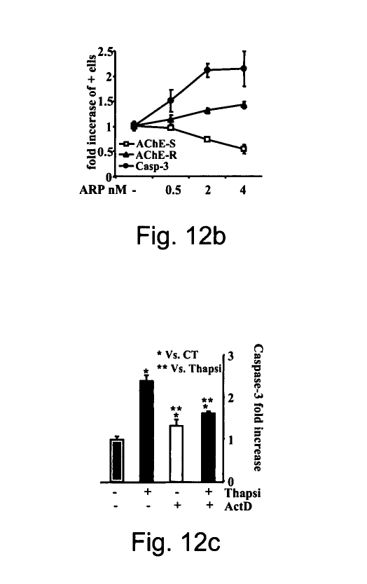

Thapsi and ARP treatments result in a splicing shift from the AChE-S splice variant to the AChE-R splice variant—Meg-01 cells were treated for 24 hours with either Thapsi or ARP (SEQ ID NO:3) and the expression of AChmiRNA and AChE transcript variants were examined. Thapsi induced a decrease in AChmiRNA (FIG. 2c) and a shift from the characteristic ACHE-S mRNA variant (SEQ ID NO:15), increasing the levels of ACHE-R mRNA variant (SEQ ID NO:16; FIGS. 8a and b). ARP-treatment also decreased the level of AChmiRNA and either BIM or H89 prevented the ARP effect (FIG. 10). Similarly, ARP increased the level of AChE-R mRNA (FIGS. 8a and b), suggesting the existence of a positive regulatory loop of ACHE alternative splicing. The increase in ACHE-R mRNA (in both Thapsi and ARP treatments) was also observed as a rightward shift in the population distribution of labeling intensity of AChE-R mRNA by FISH (FIG. 9). As is further shown in FIGS. 12a-b, immunohistochemistry analyses revealed that the increase in the level of ACHE-R mRNA induced by either Thapsi or ARP was accompanied by an increase also in the protein level (AChE-R variant, SEQ ID NO:18) and a decrease of expression of the AChE-S variant (SEQ ID NO:17), characterizing the ACHE splicing shift.

Thapsi treatment increases the incidence of activated caspase-3 positive cells—Caspases are best known for their involvement in cell death and the maturation of cytokines (Guimaraes and Linden, 2004; Shi, 2002; Thornberry and Lazebnik, 1998), but recently there have been a number of reports suggesting that caspases may have an additional role in cellular processes not related to cell death, such as lymphocyte activation and proliferation (Chun et al., 2002a; Chun et al., 2002b; Salmena et al., 2003), monocytic differentiation (Pandey et al., 2000), terminal erythroid differentiation (Zermati et al., 2001) and platelet formation (De Botton et al., 2002). Thrombopoietin-induced megakaryocytes differentiation is accompanied by caspase-9 and caspase-3 activation (De Botton et al., 2002), which induces cytoskeletal remodeling during differentiation.

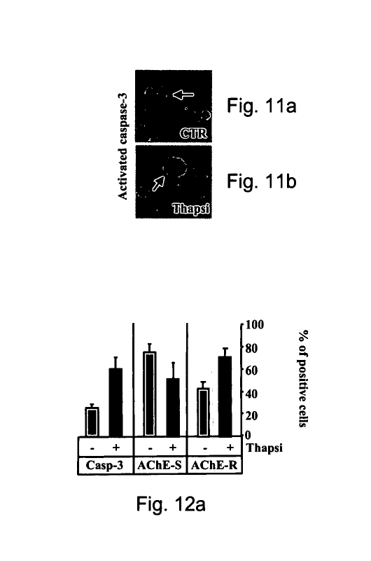

To test if calcium-induced differentiation also triggered caspase activation, Meg-01 cells were incubated with either Thapsi or ARP and the level of activated caspases was determined. As shown in FIGS. 11a-b and 12a, Thapsi treatment resulted in an increase in the incidence of activated caspase-3 positive cells. The increase in activated caspase-3 immunoreactivity was inhibited by the transcription inhibitor actinomycin D (FIG. 12c), suggesting that the Meg-01 maturation process depends on transcription.

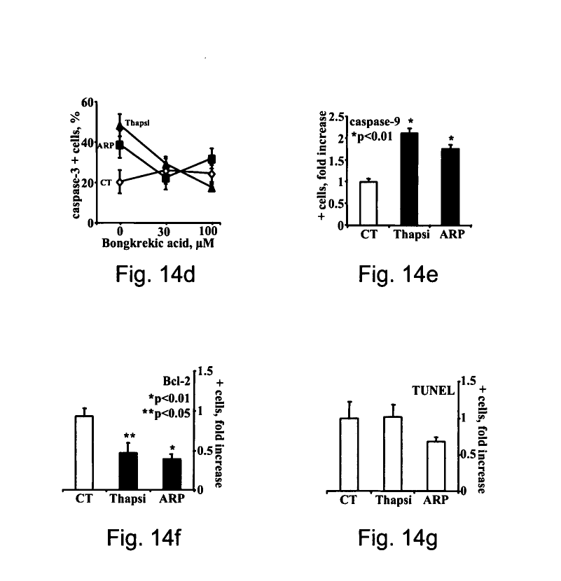

Caspase-3 activation was associated with differentiation of Meg-01 cells—As caspases often associate with cell death, the present inventors further investigated the caspase activation pathway and examined if its activation was triggering megakaryocytes cell death. Since crucial steps in cell death are characterized primarily by morphologic criteria, transmission electron microscopy was employed to observe megakaryocyte morphology. When cells were examined following 24 hours of culture in the presence of either Thapsi or ARP, differentiating megakaryocytes were recognized by the presence of initial stage demarcation membranes, which appeared as profiles of parallel membranes arising from invaginations of the plasma membrane (FIGS. 14 b and c). Although as a general rule the maturation aspects of the cytoplasm were related to the presence of a polylobed nucleus, which reflects polyploidization, a correlation between maturation of the cytoplasm and polylobation of the nucleus could not be established at this early stage. Despite the membrane blebbing, a common feature displayed by both dying cells and differentiating megakaryocytes, the observed morphology contrasts to what would be expected from degenerating cells, e.g. compacted and clumped chromatin and formation of apoptotic bodies, supporting the notion that caspase-3 activation in Meg-01 cells under Thapsi is associated with differentiation and not apopotosis.

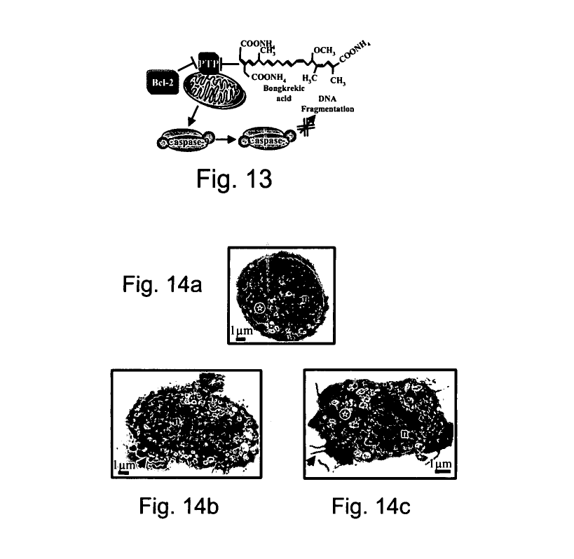

Activation of caspase-3 during megakaryocyte differentiation depends on the assembly of mitochondrial apoptosome—Caspase-3 may be activated by caspase-9. Caspase-9 activation depends on the assembly of the mitochondrial apoptosome, containing procaspase-9, APAF-1, dATP and cytochrome c. The release of cytochrome c is often associated with the opening of a permeability transition pore (PTP) in the outer membrane of the mitochondria (Budihardjo et al., 1999; Green and Reed, 1998) (FIG. 13). To test whether mitochondria participate in caspase-3 activation induced by Thapsi, the present inventors used bongkrekic acid, an inhibitor of the adenine nucleotide translocator, which is one of the components of PTP. Bongkrekic acid inhibited the caspase-3 activation induced by ER-calcium releasing or ARP (FIG. 14d), showing that activation of caspase-3 during differentiation of megakaryocytes depends on the assembly of the mitochondrial apoptosome and suggesting the involvement of prior activation of caspase-9. To test this hypothesis, immunostaining of activated caspase-9 was performed on Meg-01 cells treated with either Thapsi or ARP. As is shown in FIG. 14e, both treatments (Thapsi or ARP) increased the incidence of positive cells for activated caspase-9 immunostaining (FIG. 14e).

Bcl-2 expression decreased following Thapsi or ARP treatment—The Bcl-2 protein family includes both anti- and pro-apoptotic members, most of which act at the mitochondria. Anti-apoptotic members inhibit changes in mitochondrial homeostasis and the subsequent activation of the apoptosome signaling cascade. Consistent with over-expression of Bcl-x1, an anti-apoptotic member, leading to impaired platelet production (Kaluzhny et al., 2002), the present inventors observed a decrease in the immunoreactivity of Bcl-2, also an anti-apoptotic member, in Meg-01 cells treated with Thapsi or ARP (FIG. 14f), stressing the importance of PTP opening at the mitochondria for the signal transduction induced by calcium during megakaryocytopoiesis.

Thapsi treatment did not change the level of DNA fragmentation—To further rule out the induction of cell death by calcium signaling induced by Thapsi the TUNEL technique was employed. This assay stains in situ DNA fragmentation and is used as a hallmark of cell death. After 24 hours of incubation with either Thapsi or ARP, no significant changes were observed in the cell death baseline of the cell culture (FIG. 14g), nor even after longer periods of incubation (36 and 72 hours—data not shown).

Taken together, this implies that caspase-3 activation induced by ER-released calcium is a differentiation feature of megakaryocytes and not an apoptotic feature. Thus, a decrease in AChmiRNA effects both the balance of AChEmRNA alternative splicing products, (with an increase in the expression of AChE-R, a variant previously correlated to hematopoiesis (Chan et al., 1998; Patinkin et al., 1990; Pick et al., 2004; Soreq et al., 1994a; Soreq et al., 1994b)) and differentiation-related activation of proteases, reflecting the need for substantial cytoskeletal reorganization during this process.

### Example 3

### Synthetic AChmiON Impairs Alternative Splicing Induced by ER Calcium Release Changing the Cell Fate from Differentiation to Cell Death

Short synthetic oligonucleotides, administered directly to cell culture medium, have been shown upon internalization by the cells to specifically affect cellular processes, particularly by means of the RNA interference pathway.

ER calcium release induced by Thapsi decreased the levels of AChmiRNA and induced a splicing shift of the ACHE gene towards the ACHE-R variant. This tentatively implied that AChmiRNA impedes differentiation. To attenuate or reverse these effects, the present inventors designed a synthetic oligonucleotide [AChmiON; SEQ ID NO:23 (modified) and SEQ ID NO:1 (unmodified)] mimicking miRNA-181a in its sequence. The oligonucleotide was 2′-O-methylated (SEQ ID NO:23) to confer resistance towards nucleases and thus was suitable for direct administration into the cell culture medium. Also, the 2′-O-methyl modification tightens hybrids formed between 2′O-methylated oligonucleotides and complementary cellular mRNAs (Seidman and Soreq, 2001). Therefore, AChmiON was predicted to efficiently mimic the properties of AChmiRNA, such as hybridization with its target cellular mRNA(s) and the induction of their destruction. FIG. 15a depicts the sequence of the AChmiON oligonucleotide.

Experimental Results

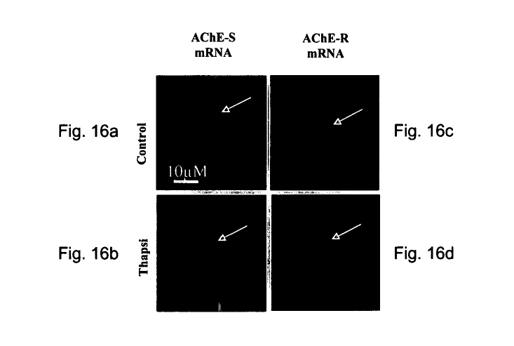

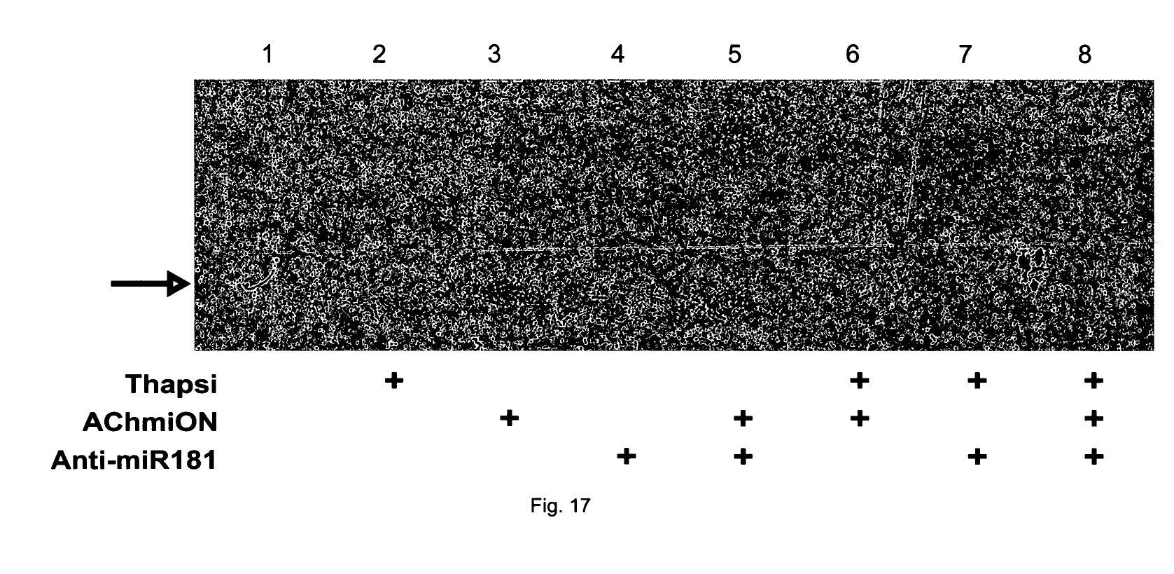

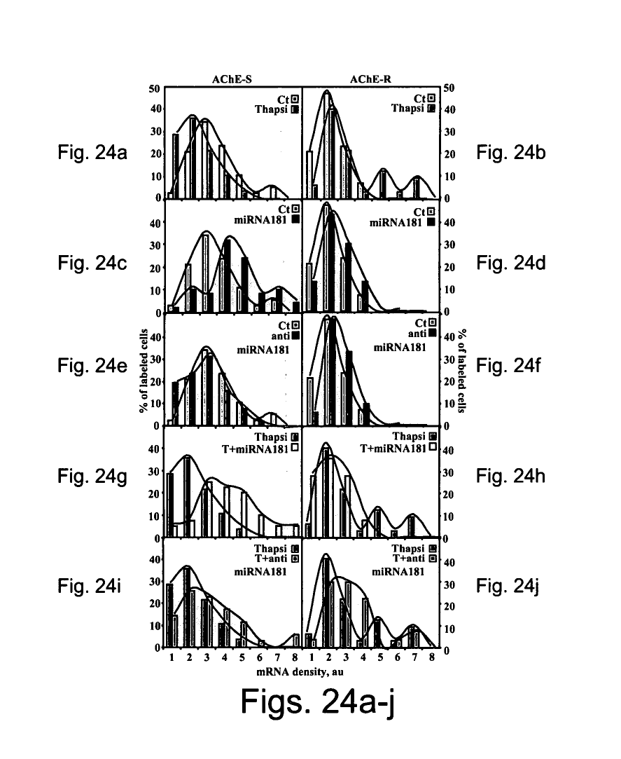

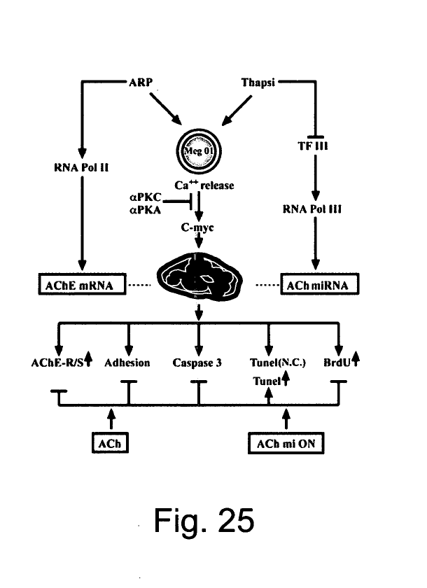

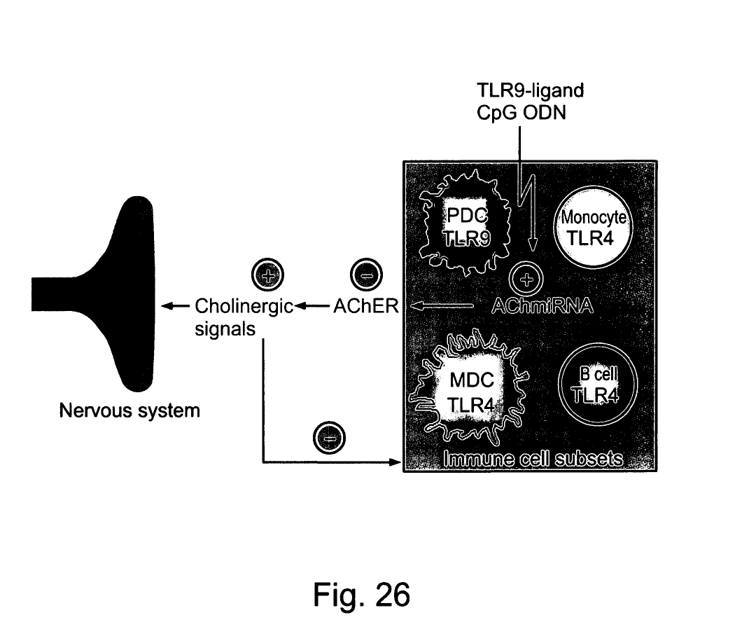

AChmiON counteracts the calcium-induced change in the ratio between AChE splice variants—Similar to the increase in AChE-R protein variant (see Example 2, hereinabove, and FIG. 12a), ER calcium release (following Thapsi treatment) induced an increase in AChE-R mRNA level and a decrease in AChE-S mRNA level (FIGS. 16a-d and 24a and b). Conversely, AChmiON (SEQ ID NO:23) increased the fraction of cells with high AChE-S levels (FIG. 24c), presumably by interfering with ACHE-R mRNA stability similar to AChmiRNA, but not with the transcriptional induction under ER Ca⁺⁺ release. As a consequence, ACHE-R mRNA was most likely over-produced, with the consequence that its levels remained unchanged (FIG. 24d). The AChmiON oligonucleotide was thus able to counteract the calcium-induced AChE splicing shift, keeping ACHE-S mRNA level above control values and inhibiting the increase in AChE-R mRNA level when both AChmiON and Thapsi were co-administered (FIGS. 24g and h). Administration of the inverse oligogonucleotide (SEQ ID NO:20) with a similar nucleotide composition which was used as a negative control, had no significant effect on the AChE splice shift (data not shown), demonstrating the sequence specificity of the AChmiON effects. Additionally, AChmiRNA targeted antisense oligonucleotide complementary to AChmiRNA (SEQ ID NO:2; modified SEQ ID NO:24) (anti-AChmiRNA) prevented the Thapsigargin-induced suppression of ACHE-S mRNA while maintaining the increase in AChE-R mRNA (FIGS. 24i-j), suggesting that this antisense sequence hybridized with its AChmiRNA target and counteracted its capacity to induce destruction of its target transcript(s), thereby ablating the capacity of AChmiRNA to destroy AChE-R mRNA. That ACHE-S mRNA levels were not reduced further suggests that the splice shift associated with the Thapsi effect was prevented. Northern Blot analysis revealed the effects of Thapsi, AChmiON (SEQ ID NO:23), and/or anti-AChmiRNA (SEQ ID NO:24) on the level of AChmiRNA (FIG. 17). As is shown in FIG. 17, anti-AChmiRNA decreased the level of anti-AChmiRNA in the presence or absence of Thapsi and/or AChmiON.

AChmiON induces apoptosis of Meg-01 cells—Increased expression of ACHE-S was shown to be associated with the induction of cell death (Zhang and Xu, 2002; Zhang et al., 2002). Therefore, the present inventors further investigated if AChmiON induction of ACHE-S correlated with cell death. TUNEL analysis of AChmiON treated cells showed an increased incidence of positive cells when compared to control and Thapsi (FIG. 15b). Such effects were also observed in the co-presence of AChmiON and Thapsi, suggesting that AChmiON counteracts the Thapsi effect while inducing apoptosis.

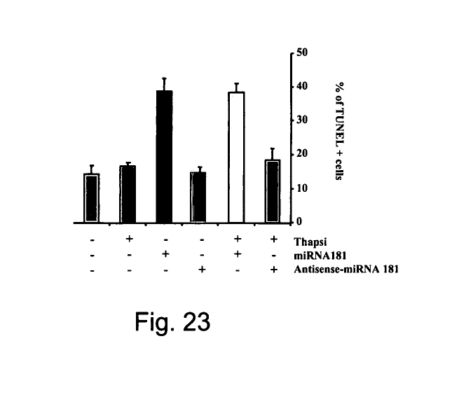

In addition, TUNEL analysis of cells treated with the AChmiON (SEQ ID NO:23) revealed that both alone, or in combination with Thapsi, AChmiON significantly increased apoptosis over control or Thapsi treated cells (FIG. 23). This may suggest utility of this or related agents for promoting apoptosis when needed.

These data suggest that AChmiRNA is an important regulator of cell fate determination, that its downregulation is required for differentiation and that its upregulation induces cell death in proliferating megakaryoblasts.

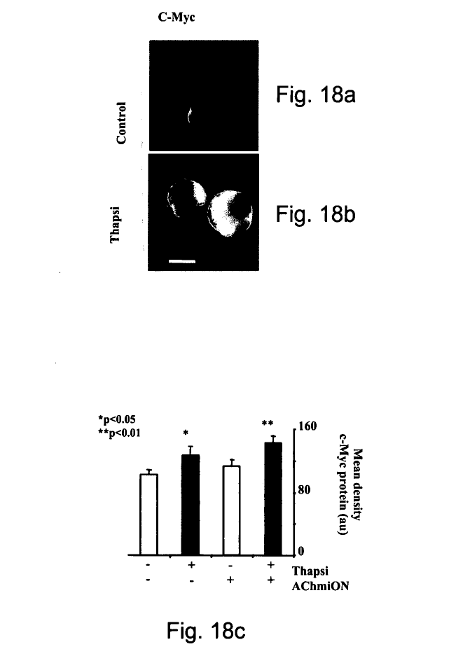

AChmiON suppressed Thapsi-induced cell adhesion but not Thapsi-induced increase of c-myc expression—Predictably, Thapsi increased adhesion of treated cells, whereas AChmiON suppressed this effect, alone or together with Thapsi (FIG. 15d). In contrast, AChmiON could not prevent the c-myc increase induced by Thapsi (FIGS. 18a-c). Thus, the AChmiON effect functions downstream from the early immediate genes reaction, but upstream from the splicing machinery.

That AChmiON prevented Thapsi effects on ACHE-R accumulation further suggested a change in the splicing variants balance, which could offer a mechanistic explanation. Indeed, the splice factor ASF/SF2 was induced by Thapsi, and AChmiON suppressed part of this effect (data not shown).

### Example 4

### The AChmiRNA Pathway Involves PKC, PKA and ACH Hydrolysis

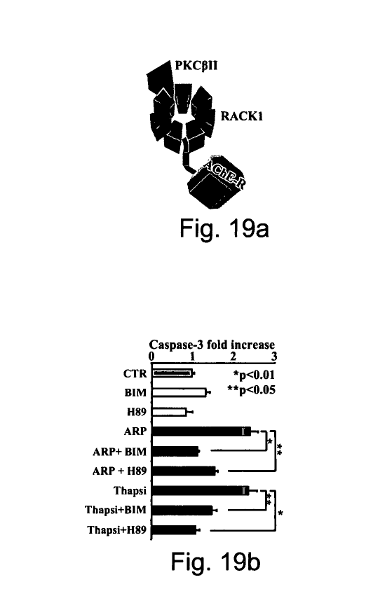

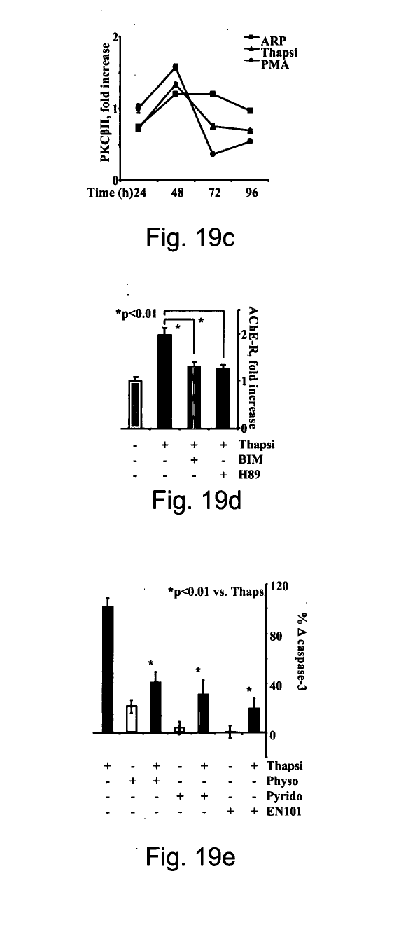

Protein kinase C (PKC) is a key component of the signaling pathways leading to proliferation and differentiation of hematopoietic cells (Marchisio et al, 1999; Oshevski et al., 1999; Racke et al., 2001). Protein kinase A as well plays a role in the proliferation and maturation of megakaryocytes (Hilden et al., 1999; Song, 1996). In brain, ACHE-R forms a triple complex with RACK1 and PKCβII (Birikh et al., 2003; FIG. 19a). The PKC inhibitor BIM and the PKA inhibitor H89 prevented the decrease in AChmiRNA levels when co-incubated with Thapsi (FIG. 2c). The involvement of PKC in the AChE-R signaling pathway has also been demonstrated in a glioblastoma model (Perry et al., 2004) (FIG. 19a). The involvement of PKC and PKA in megakaryocyte differentiation and the AChmiRNA signaling pathway was investigated, as follows.

Experimental Results

BIM and H89 prevent differentiation-associated caspase-3 activation—To further dissect the PKC-dependence of the AChmiRNA signaling pathway, Meg-01 cells were incubated with either Thapsi or ARP (SEQ ID NO:3) in the presence of the PKC inhibitor bisindolylmaleimide (BIM) (FIG. 19b). BIM inhibited the activation of caspase-3 induced by both treatments (FIG. 19b), supporting the notion that PKC is causally involved in the differentiation induction by Thapsi, ARP or PMA (positive control) (FIG. 19c). To test the participation of PKA in either the Thapsi or ARP-induced differentiation of megakaryocytes, Meg-01 cells were incubated in the presence of H89, an inhibitor of PKA. H89 prevented the differentiation-associated caspase-3 activation induced by both Thapsi and ARP (FIG. 19b). The regulation exerted by protein kinases upon this pathway appeared upstream to the ACHE splicing shift, since both BIM and H89 blocked the characteristic increase in ACHE-R immunoreactivity elicited by Thapsi or ARP (FIG. 19d).

The differentiation-associated activation of caspase-3 is blocked by AChE inhibitors—As is further shown in FIG. 20e, the differentiation-associated activation of caspase-3 is also blocked by AChE inhibitors, stressing the importance of ACh hydrolysis in the megakaryocytic differentiation process. Both physostigmine and pyridostigmine are carbamate inhibitors of acetylcholinesterase, and EN101 (SEQ ID NO:5) is a 2-O-methyl-AS-ON antisense oligodeoxynucleotide that selectively suppresses ACHE-R mRNA levels (FIG. 19e). EN101 obstructs the translation of any ACHE mRNA being generated in its presence and strongly inhibits the activation of caspase-3. Thus, these results strongly suggest that AChE alternative splicing is a central event in megakaryocytes maturation.

### Example 5

### Stress Reactions Lead to an In Vivo Decrease in AChmiRNA

Experimental Results

AChmiRNA levels decreased in mice challenged with immunological or neurological insults—In both the bone marrow and intestine of mice challenged with the cholinesterase inhibitor paraoxon, the dopaminergic poison MPTP (FIG. 20b) or the immunologically insulting bacterial lipopolysacharide (LPS; FIG. 20a), parallel and additional decreases of AChmiRNA levels to those detected during differentiation were observed. Moreover, transgenic mice overexpressing systematic AChE-R (TgR) showed sustained suppression of AChmiRNA post LPS exposure (FIG. 20a), suggesting association of AChE-R overexpression with AChmiRNA suppression. MPTP and paraoxon induced synergistic suppression in intestinal tissues (FIG. 20b), suggesting involvement with distinct target pathways (because paraoxon blocks AChE whereas the dopaminergic poison intoxicates mitochondria). This is compatible with the Bongkrekic acid data reported hereinabove. Supporting this notion, the present inventors have recently uncovered that reduced AChE functioning is associated with increased risk of Parkinsonism (Benmoyal-Segal, 2005). Thus, the effect of MPTP on AChmiRNA is likely due to the interrelationship between the cholinergic and the dopaminergic pathways.

### Example 6

### Expression of AChmiRNA in Immune Cells and Regulation by TLR9 Ligand

The organismal reaction of AChmiRNA in mice treated with LPS raised the possibility that human cells might respond similarly and if this response is mediated through the TLR (toll like receptor) system controlling the immune properties under viral or bacterial infection. To explore the possibility that this novel phenomenon of Ca⁺⁺-induced AChmiRNA suppression also occurs in primary immune cells in the peripheral blood, cultured human mononuclear blood cells were treated with CpG oligonucleotides recognized by specific TLR members and AChmiRNA levels were measured in mononuclear cells isolated from human peripheral blood.

Experimental Results

AChmiRNA were significantly increased in PBMC stimulated with the CpG A 2216 oligonucleotide—Peripheral blood mononuclear cells (PBMC) were predictably found to express AChmiRNA. The TLR9 ligand CPG-A oligonucleotide 2216 was used to stimulate immune cells. A marked increase in AChmiRNA expression was observed in PBMC upon stimulation with CPG-A 2216 (SEQ ID NO:12; FIG. 21). These data demonstrate that AChmiRNA is regulated by external signals not only in megakaryocytes but also in other hematopoietic cells such as immune cells carrying TLR ligands, and that the cholinergic system and the TLR system of pathogen recognition are causally interrelated.

Several LightCycler experiments and a recalculation of results show repetitive and consistent changes in AChmiRNA, as well as in the RNA polymerase III associated transcripts BDP1 and TBP, co-regulated with both RNA polymerase II and III. As is shown in FIG. 22, both TBP (used by both PolIII and PolII) and BDP1 (which is PolIII-specific) seem to follow the profile of the AChmiRNA amplicon, while the splicing factor ASF/SF2 showed reduced levels under both CpG ODN 2006 (SEQ ID NO:19) and CpG ODN 2216 (SEQ ID NO:12). Importantly, CpG ODN 2006, with reciprocal effects to ODN 2216 on the innate immune system, suppressed both AChmiRNA, TBP, and BDP1 in these cells, opposite to the induction effects of ODN 2216 (FIG. 22). This suggests an interrelationship between specific TLR responses and cholinergic signaling.

### Example 7

### Nitric Oxide Production as a Surrogate Marker of ACHmiON's Signaling Pathway

As demonstrated in Example 6, stimulators, such as CpG-A, of an immune response via the toll-like receptor (TLR) pathway upregulate AChmiRNA. Since production of nitric oxide is part of the non-specific cellular defense mechanism triggered by CpG-A, nitric oxide levels were examined following incubation with AChmiRNA. Due to its immune reactivity, NO is a simple molecule to trace and therefore is a preferred choice for analyzing whether AChmiRNA functions by interacting with TLRs. In addition, NO analyses enable identification of the pathways through which AchmiRNA operates, by co-addition with known stimulators of defined pathways.

Experimental Results

AChmiON oligonucleotide induces NO production from RAW 2467 cells through the JAK/STAT pathway—Murine macrophage RAW 264.7 cells were cultured in 48 well plates (50×10³ cells per well). The production of NO by these macrophages was examined following various stimuli (6 repetitions for each treatment) at three time intervals (6, 12 and 24 hours) as depicted in FIG. 27a, 27b and 27c. Treatment with bacterial lipopolysaccharide (LPS) at 1 ug/ml and/or interferon (IFN)-γ at 4 ng/ml effectively induced progressive, time-dependent production of NO. Nitrite concentration reflecting NO production levels of untreated macrophages increased from 5 to 9 to 21 μM during the tested period. In comparison, nitrite levels from macrophages treated with LPS, functioning through TLR4, increased more significantly (6, 22 and 53 μM) as did nitrite levels from macrophages treated with IFN-γ, functioning through IFN-γR, (8, 26 and 54 μM). A combination of both LPS and IFN-γ yielded 11, 33 and 68 μM, reflecting an additive function for their respective receptors. AChmiON significantly up-regulated NO production at a physiologically active concentration (i.e. at a concentration where it was shown to inhibit ACHE—100 nM). A maximal effect of 38 μM NO was reached at 24 hour post-treatment, (about 80% of the LPS effect, 53 μM). Co-stimulation with IFN-γ and AChmiON showed no additive effect, suggesting that these two agents share the same JAK/STAT signaling pathway (Bach E A, et al., Annu Rev Immunol 1997; 15:563-91). In contrast, media of macrophages treated for 24 hours with the Monarsen AS agent (human EN101) at a physiologically active concentration (100 nM) showed 6, 11 and 26 μM NO at 6, 12 and 24 hours, respectively, with insignificant change from control cells. Likewise, the inverse oligonucleotide invEN101 displayed no significant elevation in nitrite concentration, reaching 5, 9 and 22 μM values.

The response to ACmiON stimulation was slower than the response to LPS or IFN-γ implying the involvement of an additional step (e.g. destruction of specific target mRNAs). The delayed activity of AChmiON distinguishes its mode of action from those of other inducers of immune reactivity, all of which operate through direct activation of protein receptors, compatible with the concept of its RNA-targeted function.

Analysis and Discussion

While micro-RNAs are now widely accepted as important players in cellular processes, the pathways in which they are active remain mostly unknown. The present inventors describe here, for the first time, the involvement of a human miRNA, miRNA-181a (referred to herein also as AChmiRNA), in the differentiation of a megakaryocytic cell line, Meg-01. The levels of AChmiRNA decreased in correlation with differentiation induced by Thapsigargin and ARP treatments. The differentiation process was characterized by many known cellular symptoms, as well as by a splice shift between the AChE mRNA variants, S and R.

Conversely, administration of a synthetic AChmiON mimic oligonucleotide attenuated the induced differentiation process, as well as the corresponding AChE splice shift. These results suggest that AChmiRNA, and possibly other miRNAs, are involved in an early stage of the differentiation process, upstream of particular molecular events (in this case, the AChE splice shift). Notably, as the C-terminal peptide of ACHE-R, ARP, is sufficient to induce megakaryocyte differentiation, the splice shift itself must be involved in a fairly early stage of the differentiation process; this finding indicates that the contribution of AChmiRNA to the differentiation mechanism is an even earlier one.

Unraveling the contribution of miRNAs to cell fate decisions, such as differentiation, can greatly benefit the understanding of such processes. Furthermore, the fact that miRNA-like synthetic molecules, unlike the typical cellular factors, apparently undergo efficient uptake by cells (at least in culture) and perform physiological functions, implies significant prospects in therapeutic and other applications. Efforts are under way to extend the study of the effects of synthetic miRNA-like oligonucleotides to in vivo models.

An interesting question refers to AChmiRNA ability to translationally repress its target mRNAs (Doench and Sharp, 2004). The level of repression achieved depends on both the amount of the target mRNA(s) and the amount of miRNA complexes, suggesting that miRNA:mRNA interactions should be viewed in the context of other potential interactions and cellular conditions.

Several bioinformatics approaches were used to search for miRNA targets. Initial searches involved the 3′ UTR of mRNAs, subsequent algorithms used full-length cDNA sequences (Enright et al., 2003; John et al., 2004). The outcome of these searches suggests multiple mRNA targets for each miRNA (Lewis et al., 2003), with a considerable evolutionary conservation (Rajewsky and Socci, 2004). Multiple targets emerged as representing feedback loops in gene regulation, compatible with the present findings. While it is still argued whether RNA polymerase II or III transcribe miRNAs (Lee et al., 2004), several studies validated their target sequences (Kiriakidou et al., 2004; Rehmsmeier et al., 2004). One interesting target of miRNA181 is caspase-2, a mediator of neurotoxicity reactions (Troy et al., 2000). The stress-associated functions of the transcript are compatible with this prediction.

In conclusion, Micro-RNAs (miRNAs) are abundant, small, regulatory RNAs which likely play multiple roles in cell fate determination. However, the signaling processes regulating cellular miRNA levels are as yet unclear and experimental means to manipulate their levels are not yet available. The discovery that intracellular Ca⁺⁺ release in the promegakaryocytic human Meg-01 cells is accompanied by a decline in a specific miRNA sequence, miRNA-181a and that acetylcholinesterase (ACHE) inhibitors prevent this calcium-induced decline, suggests causal involvement of both cholinergic signaling and intracellular Ca⁺⁺ release in the regulation of cellular changes in this miRNA. The miRNA-181a decline was followed by a 3′ splicing shift replacing the common AChE-S splice variant with the hematopoiesis-induced variant AChE-R, further demonstrating an apparent association with the cellular balance between alternative splicing variants of the tested transcripts. Morphological hallmarks of differentiation and activation of caspase-3, a marker of megakaryocytic differentiation suggested causal involvement in the process of megakaryocytopoiesis. Both anti-AChEs and PKC or PKA inhibitors attenuated both the miRNA-181a decline and the Ca⁺⁺-induced Meg-01 differentiation, supporting this notion. Administration of AChmiON (SEQ ID NO:23), a synthetic 22-mer 2′-oxymethylated oligonucleotide mimicking the miRNA-181a sequence, blocked the Ca⁺⁺-induced differentiation effects and the modified balance between AChE mRNA splice variants while facilitating DNA fragmentation, providing a proof of concept to this hypothesis. These findings support the existence of a pathway in which cholinergic signals regulate miRNA-181a levels through intracellular Ca⁺⁺ release inducing PKC and PKA cascade(s) and suggest the use of miRNA mimics for manipulating the corresponding cellular processes.

### Example 8

### miRNAs 132 and 182* Contribute to the Inflammatory Cholinergic Reflex by Modulating Acetylcholinesterase Gene Expression

Mammalian stress reactions often impair the innate immunity pathway, to which they link through the suppression of the production of pro-inflammatory cytokines by circulating acetylcholine (ACh). Stress-induced accumulation of circulating acetylcholinesterase (ACHE) relieves this normally robust block, initiating immune reaction while increasing the risk of inflammation. In the present example, two microRNAs were identified that contribute to the termination of the inflammatory cholinergic reflex by regulating AChE activity.

Materials and Methods

Cells and cell treatment. Mouse RAW cells, human U937 cells and primary human macrophages were treated for 24 h with 1 μM CpG oligonucleotides or 1 μg/ml bacterial lipopolysaccharide added to standard growth medium

Measurement of inflammatory mediators: Interleukin 6 expression was assayed by QRT-PCR, nitric oxide was measured by Griess assay and prostaglandin E₂ levels were measured by R&D ELISA kit according to manufacturer's instructions.

MicroRNA analysis: MicroRNAs were analyzed using a spotted microRNA chip—see Example 9.

AChE activity: AChE activity was measured using Ellman's assay as described above.

RTPCR: Quantitative RTPCR was implemented to determine the change in transcript levels of IL-6 (human: forward: aaattactgaagcccacttggtt SEQ ID NO:101, reverse: actctgcaagatgccacaagg SEQ ID NO:102; mouse: forward: tagtccttcctaaccccaatttcc SEQ ID NO:103, reverse: ttggtccttagccactccttc SEQ ID NO: 104, TNF-α (human: forward: atgagcactgaaagcatgatcc SEQ ID NO:105, reverse: gagggctgattagagagaggtc SEQ ID NO:106, mouse: checked with ELISA only) following TLR4 signaling.

Results

MiRs 146, 155, 132, 182* and 212 emerged as the most relevant. miRs 132 and 182* are both predicted to be complementary to AChE and to be up-regulated by endotoxin. FIG. 28 illustrates that MiR-132 is consistently up-regulated by TLR4 signaling. MiR-382, also up-regulated in FIG. 28, is not predicted to target AChE.

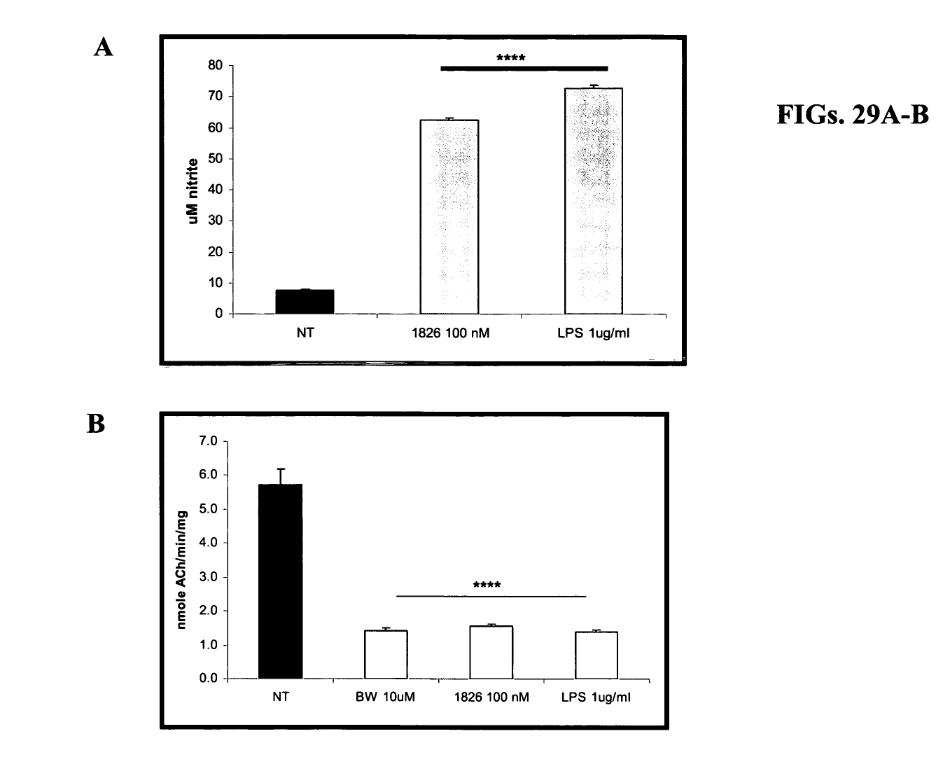

Endotoxin challenge caused a sharp induction of nitrite production as well as sharp reduction of AChE activity in macrophages—FIGS. 29A-B. The decrease in AChE activity due to endotoxin was similar to the decrease caused by 1 micromolar BW284c51 or 100 nanomolar CpG 1826, known to induce immune activities. ACHE mRNA levels were not reduced in LPS-challenged macrophages, suggesting that the miRNAs exerted translation blockade over the stress-induced Ache-R mRNA.

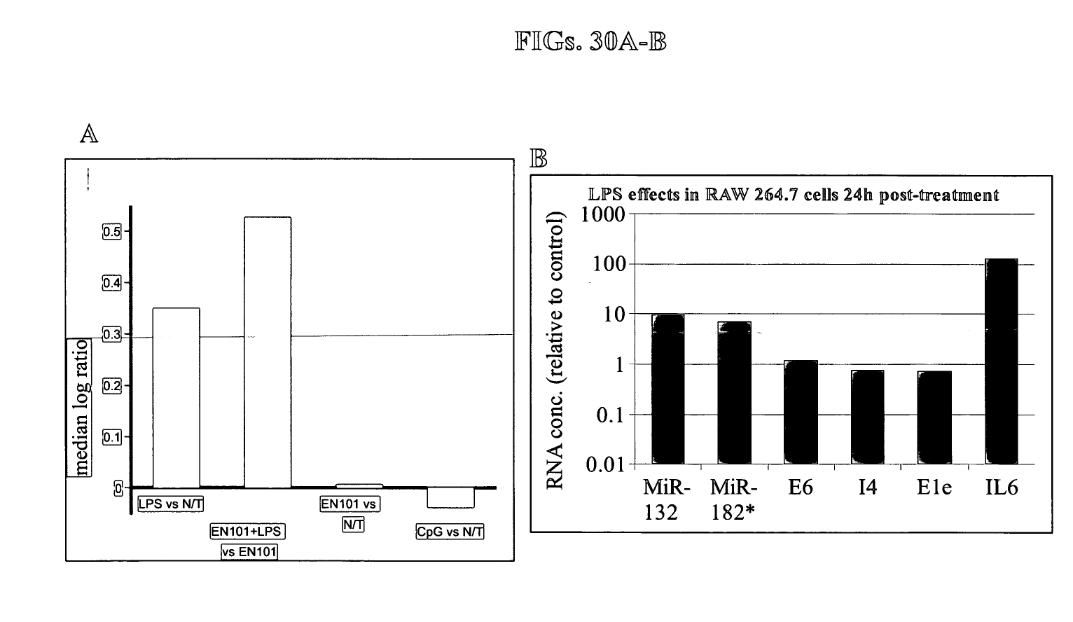

As illustrated by RT-PCR, the increase in miR-132 is specific to LPS—see FIG. 30A. Importantly, neither EN101 suppression of AChE-R mRNA levels nor CpG 1826 treatment coincided with any increase in miR132 levels. The mRNA levels of all the tested ACHE variants remained constant (FIG. 30B).

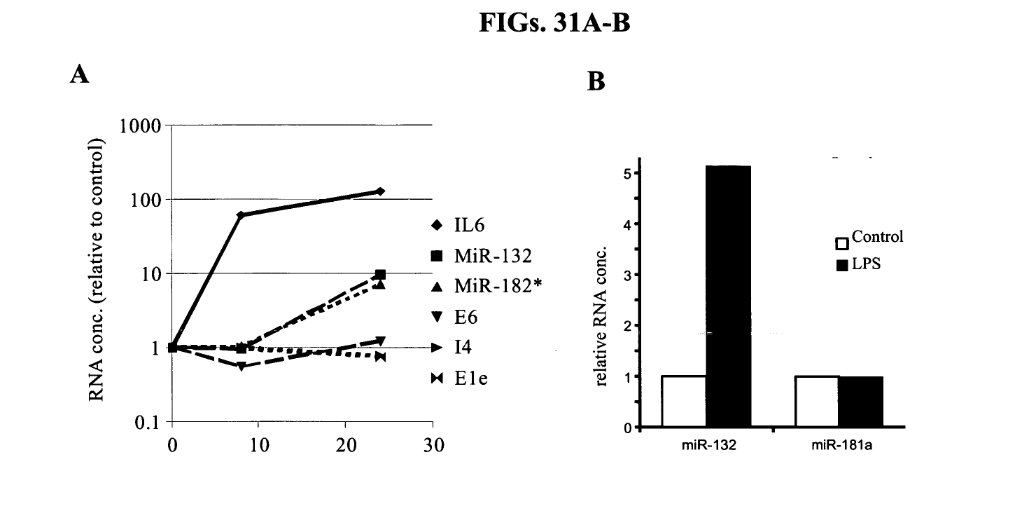

The kinetics of the reaction of miRNA mRNA increases was slower than that of the IL6 increase and took about 24 hours (FIG. 31A). Both mouse raw macrophages and human primary macrophages showed miR132 increases under IPS challenge. miR181a did not show such an increase (FIGS. 31A-B). The increase in miR132 and the corresponding decrease in its AChE target suggest causal association with translational blockade as the mechanism.

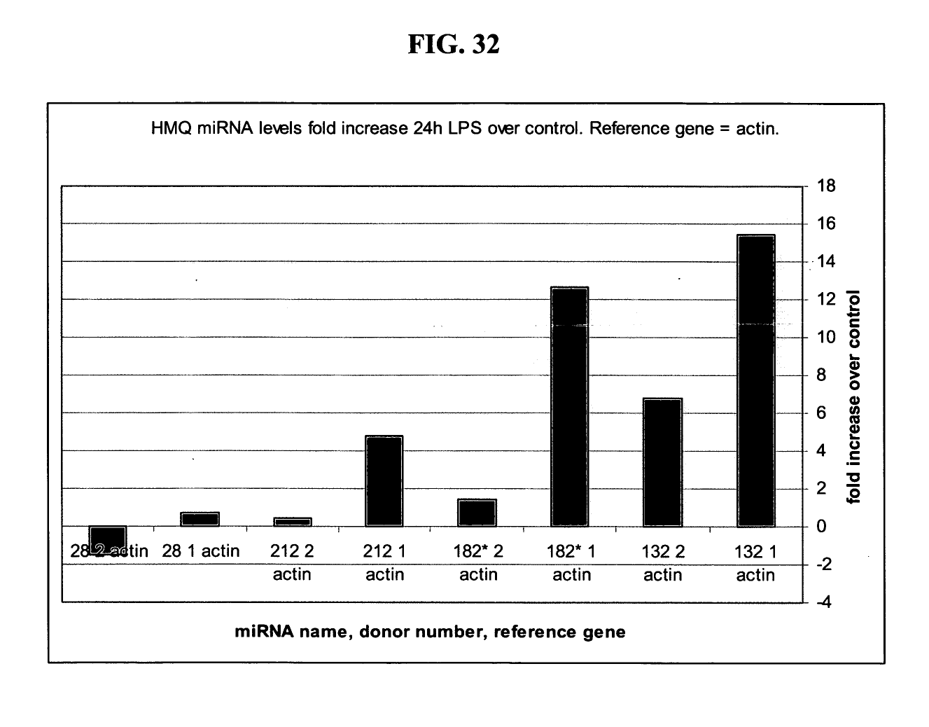

FIG. 32 illustrates the results of a quantitative RT-PCR analysis using a primer-extention PCR protocol and LNA-modified primers (as described by Raymond C K, et al., RNA. 2005 November; 11(11):1737-44.) (sequences for miR-132: forward: UAA+CA+GUCUACAGCC, RT gene-specific: catgatcagctgggccaaga CGACCATGGCTG, universal reverse primer: catgatcagctgggccaaga).

It can be seen that microRNAs 132, 182* and 212 are consistently up-regulated following TLR4 challenge in human primary cultured macrophages.

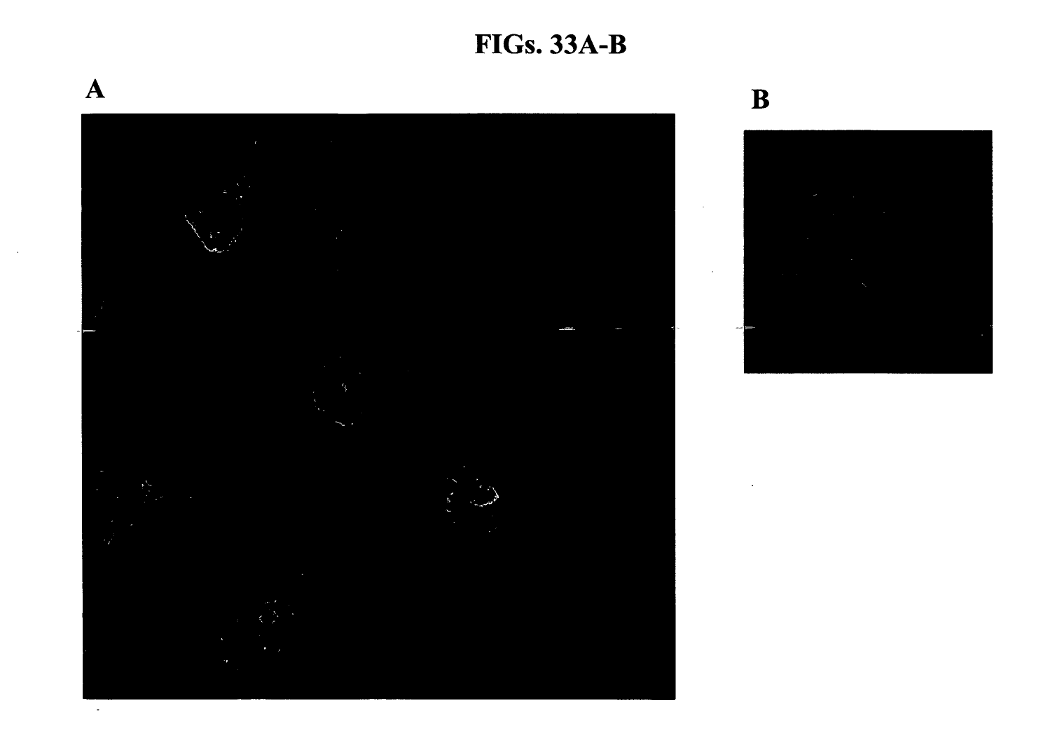

In situ hybridization experiments show the enhanced expression of microRNAs 132 following TLR4 challenge in human primary macrophages (FIGS. 33A-B).

Conclusion

These findings support the notion that miRs 132 and 182* contribute to the cholinergic suppression of inflammatory stress, complementing the reported capacity of miR146 to inhibit the TLR-mediated innate immune responses [Taganov, K. D., et al., (2006) Proc Natl Acad Sci USA 103, 12481-6]. The observed changes thus highlight evolutionarily conserved interrelations between the stress-activated macrophage reactions of ACh, TLR and miRs and suggest the use of RNA-targeted control over inflammatory reactions.

### Example 9

### The Effects of Immunogenic Activation on the Expression of Macrophage miRNAs

To examine the effects of immunogenic activation on the expression of macrophage miRNAs, an in-house spotted array was constructed and hybridized with RNA samples from primary human macrophages. Cells were isolated from buffy coats from healthy donors, underwent pooling that elicited mixed leukocyte response (MLR) and subjected in vitro to several distinct agents or combinations thereof. Following treatment, RNA was purified and analyzed.

Materials and Methods

Cells and cell treatments: Macrophages were isolated from buffy coats from healthy donors, underwent pooling that elicited mixed leukocyte response (MLR) and subjected in vitro to several distinct agents or combinations thereof, some known to elicit macrophage activation (listed in Table 2, hereinbelow).

TABLE 2

| Name | Description | Dose | Comments |
|---|---|---|---|
| LPS | Gram-negative | 1 μg/ml | Known to be |
| | bacterial | | recognized by TLR-4 |
| | lipopolysacharide | | Toll-like receptor and |
| | (endotoxin) | | MD2 to induce |
| | | | cytokine and |
| | | | interferon responses |
| CpG | CpG ODN 2006 | 1 μM | Recognized by TLR9 |
| | (Type B) | | |
| EN101 | Antisense oligo for | 1 μM | |
| | AChE-R (Monarsen) | | |

Microarray Method: The mirVana (Ambion, Austin, Tex.) oligonucleotide set was used to construct the in-house array. The microarray carried 200 spotted probes complementary to known human and mouse miRNAs. To compose the array, the mirVana probeset was dissolved in 3×SSC to a final concentration of 20 mM, and printed on Ultragaps slides (Corning, Corning, N.Y.), using the MicroGrid spotter (Genomic Solutions, Holliston, Mass.). The array layout contained 12 subgrids, each composed of 11 rows and 12 columns. Each oligonucleotide was spotted 6 times on the array. The experiments were designed for comparison of two samples. Data was therefore always relative rather than absolute. In these experiments, a “reference design” (Churchill, 2002, Nature genetics, 32, 490-495) was used, in which RNA from cells or tissues is compared. In addition, dye-swapping tests were performed, aimed to exclude dye-specific labeling differences (Dombkowski et al., 2004, FEBS Lett, 560, 120-124). Labeling was performed using the CyDye reactive dye pack (Amersham, NSW, Australia), as instructed. Pre-hybridization was in pre-heated 5×SSC, 1% BSA, 0.1% SDS solution, at 42° C. for 45 min. Cy3 and Cy5-labeled fragmented RNA (3 ug each) were added to the hybridization solution (3×SSC, 0.1% SDS, 10 μg polyA, 20 μg tRNA), heated at 95° C. for 4 minutes for eliminating secondary structures and applied to the slides in hybridization chambers (Corning, N.Y., USA) for 15 h at 64° C. Hybridized slides were successively washed in: 1×SSC, 0.1% SDS (5 min); 0.1×SSC, 0.1% SDS (5 min) and 0.1×SSC (3×1.5 min) and were dried by centrifugation (˜1000 g).

Scanning and quantification: This was adapted from (Ben-Ari S, et al., J Neurochem. 2006 April; 97 Suppl 1:24-34.) Briefly, an Affymetrix 428 Array Scanner was used at the maximum gain setting, at the wavelengths of 532 nm and 650 nm. Scans were controlled by the “Jaguar” software (Affymetrix, CA, USA), saved as TIFF files and exported to the “Imagene” program (Biodiscovery Inc, CA, USA) to define the spots, convert the intensity signals into numbers and calculate quality parameters such as “shape regularity”, “empty spot” etc. Data normalization, exclusion of unreliable spots and combining the information from all 4 slides were performed in an in-house Matlab program. First, median background was subtracted from the median signal intensity, to normalize background noises. Background area was defined as a circle of 5 pixels diameter separated from the signal area by 4 pixels. In addition, “background contaminations” (specific pixels identified by the software as outliers) were eliminated. Spot quality was examined according to pre-defined Imagene's parameters, standard deviation of pixels intensity in each spot etc. Spots with intensities below the threshold derived from the negative control spots or with saturated pixel values were excluded. The Cy3 and Cy5 signal intensities of each spot were normalized to the mean Cy3 and Cy5 intensities in the slide. For each spot, an initial Cy3/Cy5 ratio has been calculated, and was transformed to a log2 basis. This value was designated LR (log2 ratio). The bias resulting from dependence of the Cy3/Cy5 ratios on signal intensities had been corrected using the locally weighted scatter-plot smoothing (LOWESS) algorithm (Quackenbush, 2002), yielding new LR for each spot. Then, results from all valid spots (up to 6) representing the same oligonucleotide were combined. Probes with less than 3 valid spots were excluded, and median and mean LR were calculated.

Identification of miRNAs showing significantly changed expression levels: miRNAs, the expression levels of which were significantly altered were identified by the discrete approach (Ben-Shaul et al., 2005, Bioinformatics, 21, 1129-1137). A threshold was set for identifying changed transcripts which were not disqualified due to any quality parameter and which showed LR>0.25 or <−0.25 values, with a P-value of the sign-test smaller then 0.05.

Covariance between treatments was calculated in OpenOffice 1.1 for Mac OS X, using the entire miRNA collection represented on the chip (for signals that failed the MatLab analysis, a Median LR value of 0 was assigned).

Results

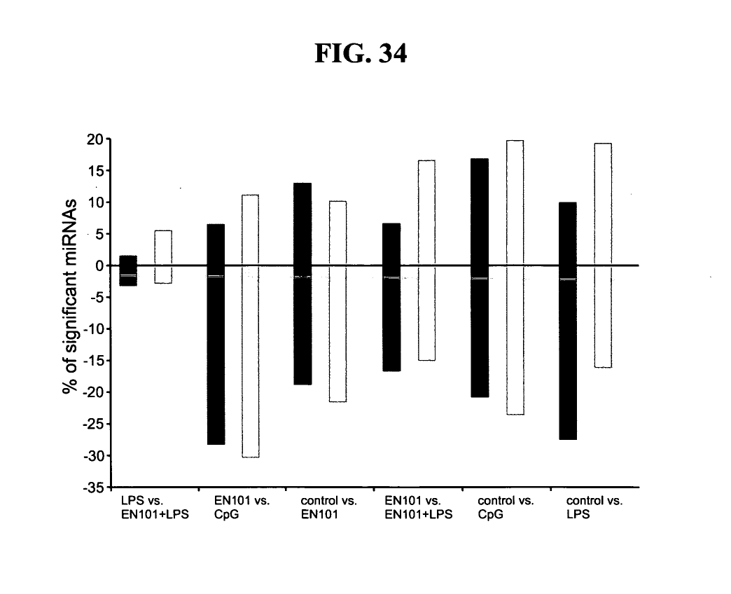

Discrete analysis: The treatments differ in the extent of change they inflict on miRNA expression. Specifically, the CpG oligo caused the greatest change as measured by the total number of miRNAs that changed beyond the set threshold of median LR=0.25, divided by the total number of significant transcripts (FIG. 34); even more than LPS, which was comparable to EN-101 in the extent of induced change.

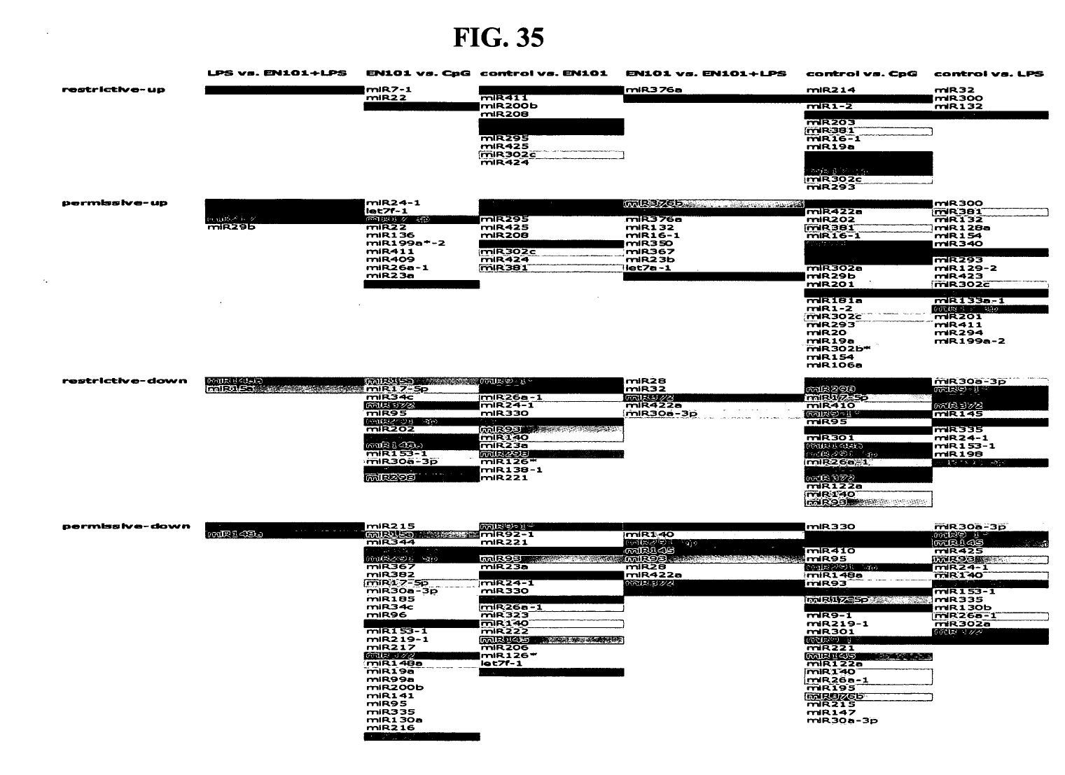

FIG. 35, lists the miRNAs with a mean LR change of 0.25 or more in absolute value. miRNAs that recurred in different comparisons are marked in colors for ease of location on the table. (Spots where only one of the dyes could be detected, were omitted for the stringent test but included in the permissive; thus the calculated LR values of the permissive analysis are meaningless, but the trend indications may be more comprehensive than in the stringent analysis.)

The effects of LPS and EN-101 on the miRNA profile appear to be mediated by the same mechanism judging by the relative lack of additivity of the combined effects (FIG. 34). In contrast, the CpG oligo and EN-101 have seemingly independent, and divergent, effects on miRNA expression (FIGS. 34-35).

Several miRNAs show unique patterns of regulation by the different treatments, while others are more universally affected (FIG. 35). Examples:
miR-183 was up-regulated, and miR-185 down-regulated, by the two different oligonucleotide treatments, but not by LPS;
miR-30a-3p and miR-372, a Bcl-2-suppressing oncogene (Voorhoeve et al., 2006, Cell. 2006 Mar. 24; 124(6):1169-81), were consistently down-regulated by LPS and CpG, but not by EN-101.
miR-17-5p, reported to be regulated by c-myc and to down-regulate E2F (O'Donnell et al., 2005, Nature. 2005 Jun. 9; 435(7043):839-43), and overexpressed in solid tumors (Volinia et al., 2006, Proc Natl Acad Sci USA. 2006 Feb. 14; 103(7):2257-61), was down-regulated by CpG alone.
miR-9-1* was down-regulated, while miR-302a and miR-381 were up-regulated, by all three treatments relative to control.

These results suggest a role in innate immunity for several miRNAs with hitherto unknown function, as well as several miRNAs with previously reported involvement in cell growth and death, tissue development and oncogenesis.

Continuous analysis: To compare the overall impacts of the various treatments on the miRNA profile, the covariance between every 2 data sets (each resulting from a comparison between 2 samples) was calculated (FIG. 35). Covariance gives a measure to how similar is the effect of 2 treatments on the entire miRNA profile; it is increased when the same miRNAs are up- or down-regulated by both treatments to a similar extent, while random changes (“noise”) bring the covariance closer to the zero value. A negative covariance thus indicates opposite effects by the 2 compared treatments (as if one of them up-regulates a group of miRNAs and the other down-regulates them).

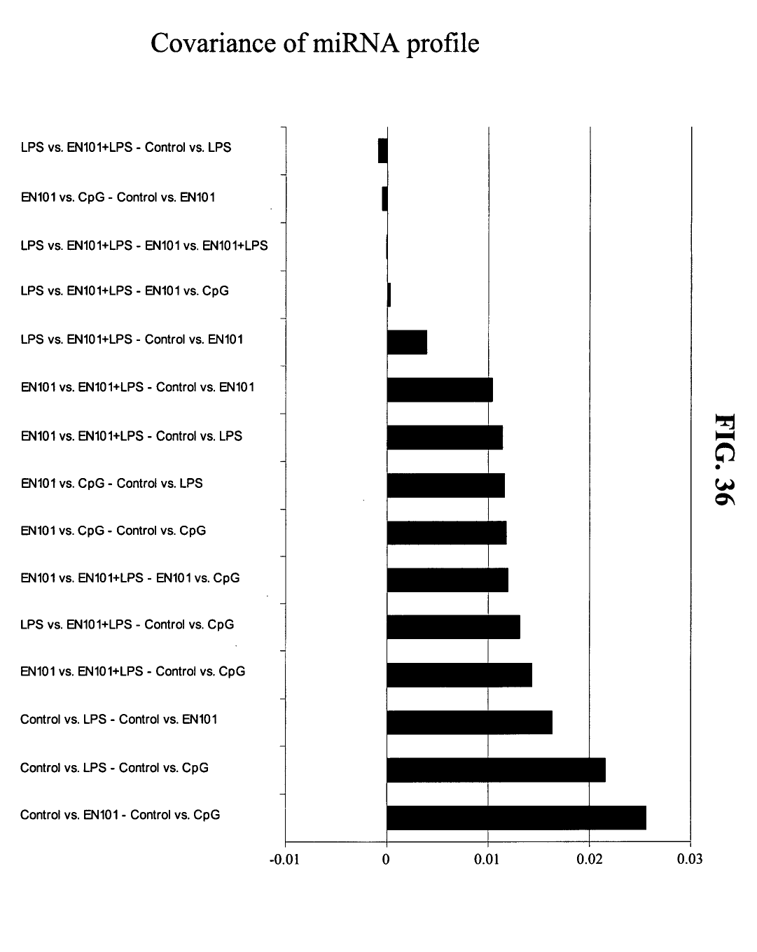

As seen in FIG. 36, EN101 and CpG invoke the closest effects on the miRNA profile from all the compared treatments. This suggests that in the cell population analyzed, EN101 acts primarily as a TLR agonist, putatively acting through TLR9. Indeed, a conserved CpG motif is contained in the human EN101 sequence: 5′CTGCCACGTTCTCCTGCACC3′. In contrast, the murine EN101 sequence contains no CpG motifs, and the murine oligo had in fact failed to activate the murine RAW 264 macrophage line, as measured by Nitric Oxide production.

Notably, none of the compared treatments had opposite effects on the miRNA profile; rather, most of the treatments caused somewhat similar effects (which probably relate to the reinforcement of the pro-inflammatory response in the already-activated macrophages), and some treatments showed non-related/independent effects, giving a covariance value close to zero.

Identification of miRNAs putatively involved in TLR9 signaling: Combining the discrete and continuous approaches described above, a list of miRNAs which were similarly impacted by both CpG and EN101 oligos, but not by the other immunostimulatory treatments may be compiled. Thus, both oligos up-regulated miR-183, 292-3p, 302c, 361, and 381, (of which only miR-292-3p appears to be up-regulated only by the oligos); and down-regulated miR-9-1*, 26a-1, 27a, 33, 93, 140, 145, 185, 221, and 298, (of which miR-185, 221 appear to be down-regulated exclusively by the oligos). These 3 miRNAs, miR-292-3p, 185 and 221, seem to be specifically involved in TLR9 signaling; although the other miRNAs mentioned above are all involved in the course of the pro-inflammatory response. MiR-221 has been shown to inhibit normal erythropoiesis and erythroleukemic cell growth via kit receptor down-regulation (Felli et al., 2005, Proc Natl Acad Sci USA. 102(50):18081-6) and is up-regulated in primary glioblastoma (Ciafre et al., 2005, Biochem Biophys Res Commun. 334(4):1351-8); the other two miRNAs have no other reported function to date.

### Example 10

### The Effects of Chronic Stress on miRNA

Mice were subjected to immobilization stress either acutely or repeatedly, to create chronic stress. Brain regions were taken from which rNA was extracted and subjected to spotted microarray miR analysis (the microarray of Example 9). miR profiling in the brain was performed in both rat and mouse brain. Comparisons involved acute to chronic stress, short to long and brain regions.

Results

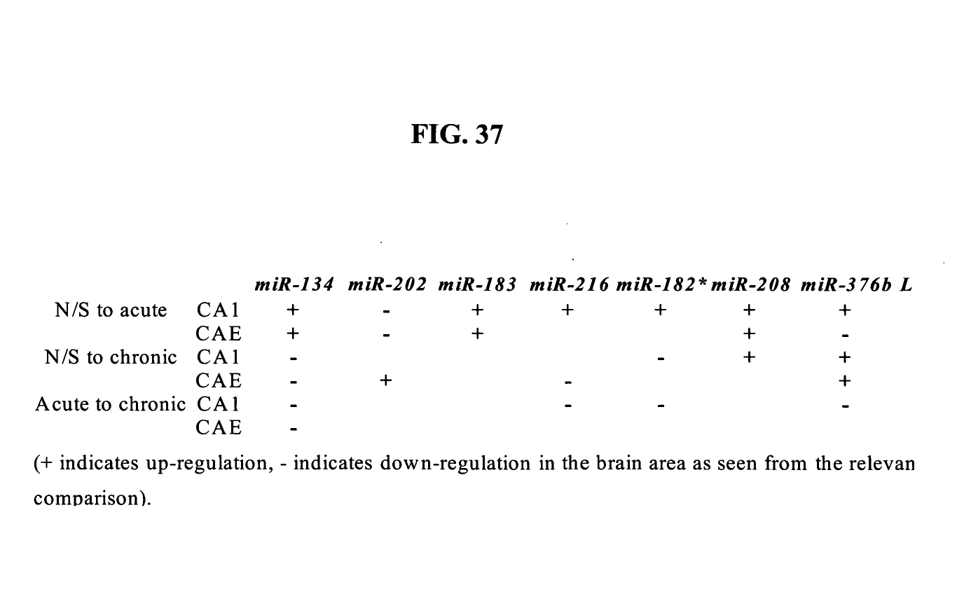

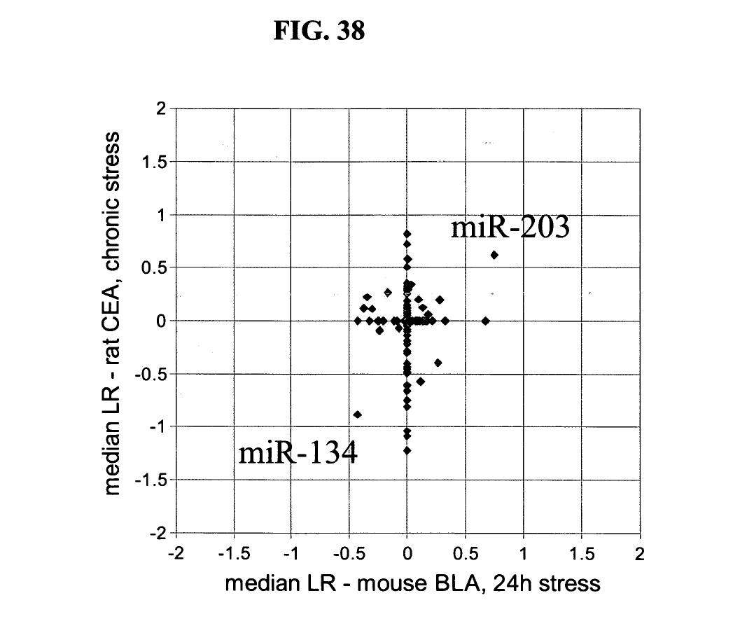

FIG. 37 summarizes the outcome. CA1=hippocampal CA1, BLA=amygdala. FIG. 38 provides MiRs comparison across samples, species and treatments through median LR comparison and illustrates that prolonged stress upregulates miR-203, downregulates miR-134 in both mouse and rat.

The working hypothesis which emerges is as follows: receptors for external stimuli, including but not limited to TLRs and/or ACh receptors, induce changes in the profile of miRs which in turn control the levels of splicing factors by suppressing translation of the mRNAs encoding these factors. This in turn changes the splice variants of target transcripts, including that of aChE-R mRNA which leads to a cellular reaction to that stimulus.

The summary of the brain experiment is as follows: chronic stress exerts a stronger change in miR composition than acute stress; several miRs show unique patterns of regulation by acute and chronic stress in different regions of the brain; at least one of those, miR 134, is consistently upregulated in acute stress and downregulated in chronic stress, in both mouse and rat. Importantly, its predicted targets include SC35, a major splicing factor which we found to be up-regulated for several weeks at least in the pre-frontal cortex of chronically stressed mice (Meshorer, Mol Psych 2005). SC35 is a pivotal element in splicing of the AChE-R mRNA. On the Splicechip, SC35 showed an inverse pattern of regulation to that of miR134, supporting the notion of its involvement in the mir-mediated control of stress reactions.

### Example 11

### Transfection with Mimics of AChE-Targeting miRs Attenuate Macrophage Activation

Cells—Murine RAW 264.7 macrophage-derived cell line known to express TLR4 and respond to endotoxin by production of pro-inflammatory mediators such as Nitric Oxide (NO), prostaglandins, and cytokines as IL1, IL6, and TNFalpha were used (described in the preceding examples e.g., Examples 7 and 8). Cells were grown in 5 ml flasks in a humidified atmosphere in Dulbecco's modified Eagle's medium (DMEM, Biological Industries) supplemented with 10% fetal calf serum (FCS) and 2 mM L-glutamine at 37° C., 5% CO₂.

NO detection—Griess method was used to determine NO₂ levels in medium in which macrophages had been cultured. 100 μl of each sample+100 μl of Griess reagent (1% sulfanilamide/0.1% naphthylethylenediamine dihydrochloride/2.5% H₃PO₄)—were incubated 10-20 min at room temperature. Read absorbance at 546 nm in micro-ELISA reader.

Statistics—Two-tailed Student's t-test was employed for comparisons between 2 groups or more. P values <0.05 were considered significant.

Synthesis of miR mimetics—miR 132 and 182* (SEQ ID NOs: 54 and 93, respectively) were LNA modified (products are designated with SEQ ID NOs. 108 and 108, respectively). A locked nucleic acid (LNA), often referred to as inaccessible RNA, is a modified RNA. Ribose moiety of LNA nucleotide is modified with an extra bridge connecting 2′ and 4′ carbons. The bridge “locks” the ribose in 3′-endo structural conformation, which is often found in A-form of DNA or RNA. LNA nucleotides can be mixed with DNA or RNA bases in the oligonucleotide whenever desired. The locked ribose conformation enhances base stacking and backbone pre-organization. This increases significantly the thermal stability of the ODN (You et al. 2006 Nucleic Acids Re May 2; 34(8):e60, Herein incorporated by reference in its entirety).

TABLE 3

| | | |
|---|---|---|
| miR | Backbone/SEQ ID NO: | LNA Modified/SEQ ID NO: |

| | | |
|---|---|---|
| 182* | ugguucuagacuugccaacua/93 | 5′Tg+gTT+CTA+TAC+gAC+TTg+CCA+ACT+A3′/108 |
| 132 | Uaacagucuacagccauggucg/54 | 5′TA+ACA+gTC+TAC+AgC+CAT+ggT+CTg3′/107 |

+-LNA modification

Cell transfection—electroporation and lipofection were used for cell transfection.

Results

Production of nitric oxide is dramatically increased in macrophages following endotoxin treatment (e.g. in RAW 264.7 cells—FIG. 29a). To check if miR-132 and miR-182* (also predicted to bind the 3′ UTR of AChE can directly influence the response to endotoxin, synthetic LNA (locked nucleic acid)-modified oligos mimicking the sequence of these miRs (as well as control oligos with scrambled sequence) were transfected or electroporated into RAW 264.7 cells. The transfected cells were challenged with endotoxin and their NO production was measured. The lowered NO production following transfection suggests that miR-132 and miR-182*-mimicking oligos significantly attenuate endotoxin response (FIG. 39).

### Example 12

### Synthetic Oligo Mimicking miR-132 Suppresses AChE mRNA Levels, while a Reverse-Complementary “Anti-miR” Up-Regulates AChE-S mRNA

Materials and Methods

Cells—Primary human macrophages obtained from donors using methods which are well known in the art.

miR Reverese complementary sequence—anti132 is set forth in CG+ACC+ATG+GCT+GTA+GAC+TGT+TA (SEQ ID NO:109), anti182 is set forth in 5′T+AgT+Tgg+CAA+gTC+TAg+AAC+CA3′, whereby LNA modification is marked as + before the modified nucleotide.

Quantitative RT-PCR—RT was performed using SuperScript III kit reagents (Invitrogen) as per kit instructions, and ABI-7900HT (Applied Biosystems) equipped with dedicated software (ver. 2) was used to amplify the resulting cDNA template (SYBR Green I RNA amplification kit, Roche). Triplicate values of each treatment were normalized to β-actin mRNA.

NO detection—Was effected as described in Example 11 above.

Results

To model the direct effects of miR-132 on AChE in primary human macrophages, they were transfected with synthetic LNA-modified oligos either mimicking the sequence of miR-132 (mimic-132) or having the reverse-complementary sequence (anti-132). QRT-PCR analysis showed (FIG. 40) that mimic-132 caused a dramatic decline in the levels of both ACHE-S and AChE-R mRNA. Furthermore, anti-132 caused an increase in mRNA levels, albeit of the ACHE-S variant alone. In addition, anti-132 led to up-regulation of Inducible Nitric Oxide Synthetase (iNOS), in agreement with the suggested immuno-suppressive function of the endogenous miR-132.

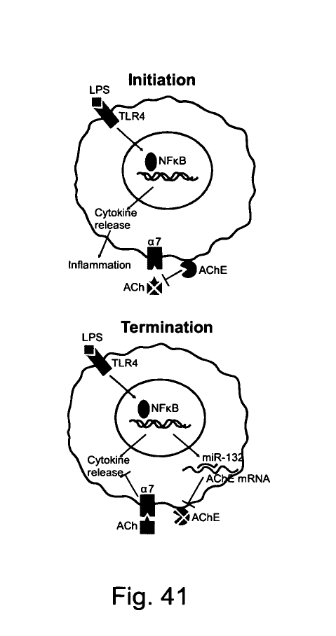

The modelled activity of AChE directed miRNAs is illustrated in FIG. 41. Without being bound to theory, the mechanism is suggested to act akin to an intrinsic timer of innate immunity cells, which causes delayed upregulation of miRs that suppress pro-inflammatory factors (in this case ACHE) by binding their mRNA and inhibiting translation. The decrease in AChE activity leads to higher levels of secreted Ach in the cell's environment, which in turn suppress the inflammation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

Adams, J. M. and Cory, S. (1998). The Bcl-2 protein family: arbiters of cell survival. Science 281, 1322-1326.
Andres, C., Beeri, R., Friedman, A., Lev-Lehman, E., Henis, S., Timberg, R., Shani, M., and Soreq, H. (1997). Acetylcholinesterase-transgenic mice display embryonic modulations in spinal cord choline acetyltransferase and neurexin Ibeta gene expression followed by late-onset neuromotor deterioration. Proc Natl Acad Sci USA 94, 8173-8178.
Ballas, Z. K., Krieg, A. M., Warren, T., Rasmussen, W., Davis, H. L., Waldschmidt, M., and Weiner, G. J. (2001). Divergent therapeutic and immunologic effects of oligodeoxynucleotides with distinct CpG motifs. J Immunol 167, 4878-4886.
Bayer, E. and Mutter, M (1972). Liquid phase synthesis of peptides. Nature 237(5357), 512-513.
Beelman, C. A. and Parker, R. (1995). Degradation of mRNA in eukaryotes. Cell 81, 179-183.
Beeri, R., Le Novere, N., Mervis, R., Huberman, T., Grauer, E., Changeux, J. P., and Soreq, H. (1997). Enhanced hemicholinium binding and attenuated dendrite branching in cognitively impaired acetylcholinesterase-transgenic mice. J Neurochem 69, 2441-2451.
Bernstein, E., Denli, A. M., and Hannon, G. J. (2001). The rest is silence. RNA 7, 1509-1521.
Birikh, K., Sklan, E., Shoham, S., and Soreq, H. (2003). Interaction of “readthrough” acetylcholinesterase with RACK1 and PKCbetaII correlates with intensified fear-induced conflict behavior. Proc Natl Acad Sci USA 100, 283-288.
Brown, L. M., Blair, A., Gibson, R., Everett, G. D., Cantor, K. P., Schuman, L. M., Burmeister, L. F., Van Lier, S. F., and Dick, F. (1990). Pesticide exposures and other agricultural risk factors for leukemia among men in Iowa and Minnesota. Cancer Res 50, 6585-6591.
Budihardjo, I., Oliver, H., Lutter, M., Luo, X., and Wang, X. (1999). Biochemical pathways of caspase activation during apoptosis. Annu Rev Cell Dev Biol 15, 269-290.
Chan, P., Cardy, R., Haseman, J., Moe, J., and Huff, J. (1994). Leukemia induced in rats but not mice by dimethyl morpholinophosphoramidate, a stimulant anticholinesterase agent. Toxicology 91, 127-137.
Chen, C. Z., Li, L., Lodish, H. F., and Bartel, D. P. (2004). Micro-RNAs modulate hematopoietic lineage differentiation. Science 303, 83-86.
Cohen, O., Erb, C., Ginzberg, D., Pollak, Y., Seidman, S., Shoham, S., Yirmiya, R., and Soreq, H. (2002). Neuronal overexpression of ‘readthrough’ acetylcholinesterase is associated with antisense-suppressible behavioral impairments. Mol Psychiatry 7, 874-885.
Cohen, O., Reichenberg, A., Perry, C., Ginzberg, D., Pollmacher, T., Soreq, H., and Yimmiya, R. (2003). Endotoxin-induced changes in human working and declarative memory associate with cleavage of plasma “readthrough” acetylcholinesterase. J Mol Neurosci 21, 199-212.
de Botton, S., Sabri, S., Daugas, E., Zermati, Y., Guidotti, J. E., Hermine, O., Kroemer, G., Vainchenker, W., and Debili, N. (2002). Platelet formation is the consequence of caspase activation within megakaryocytes. Blood 100, 1310-1317.
de Klein, A., van Kessel, A. G., Grosveld, G., Bartram, C. R., Hagemeijer, A., Bootsma, D., Spurr, N. K., Heisterkamp, N., Groffen, J., and Stephenson, J. R. (1982). A cellular oncogene is translocated to the Philadelphia chromosome in chronic myelocytic leukaemia. Nature 300, 765-767.
Deutsch, V. R., Pick, M., Perry, C., Grisaru, D., Hemo, Y., Golan-Hadari, D., Grant, A., Eldor, A., and Soreq, H. (2002). The stress-associated acetylcholinesterase variant ACHE-R is expressed in human CD34(+) hematopoietic progenitors and its C-terminal peptide ARP promotes their proliferation. Exp Hematol 30, 1153-1161.
Donze, O. and Picard, D. (2002). RNA interference in mammalian cells using siRNAs synthesized with T7 RNA polymerase. Nucleic Acids Res 30, e46.
Elbashir, S. M., Martinez, J., Patkaniowska, A., Lendeckel, W., and Tuschl, T. (2001). Functional anatomy of siRNAs for mediating efficient RNAi in Drosophila melanogaster embryo lysate. Embo J 20, 6877-6888.
Ellman, G. L., Courtney, D., Andres, V. J., and Featherstone, R. M. (1961). A new and rapid colorimetric determination of acetylcholinesterase activity. Biochem Pharmacol 7, 88-95.
Farchi, N., Soreq, H., and Hochner, B. (2003). Chronic acetylcholinesterase overexpression induces multilevelled aberrations in mouse neuromuscular physiology. J Physiol 546, 165-173.
Gewirtz, A. M. (1999). Oligonucleotide therapeutics: clothing the emperor. Curr Opin Mol Ther 1, 297-306.
Gewirtz, A. M. (2000). Oligonucleotide therapeutics: a step forward. J Clinical Oncology 18, 1809-1811.
Gewirtz, A. M., Sokol, D. L., and Ratajczak, M. Z. (1998). Nucleic acid therapeutics: state of the art and future prospects. Blood 92, 712-736.
Green, D. R. and Reed, J. C. (1998). Mitochondria and apoptosis. Science 281, 1309-1312.
Gresham, D. (2003). Research Notes: “A function for mammalian micro-RNA”. Nature Genetics 34, 250.
Grisaru, D., Deutch, V., Shapira, M., Galyam, N., Lessing, B., Eldor, A., and Soreq, H. (2001). ARP, a peptide derived from the stress-associated acetylcholinesterase variant, has hematopoietic growth promoting activities. Mol Med 7, 93-105.
Grisaru, D., Lev-Lehman, E., Shapira, M., Chaikin, E., Lessing, J. B., Eldor, A., Eckstein, F., and Soreq, H. (1999). Human osteogenesis involves differentiation-dependent increases in the morphogenically active 3′ alternative splicing variant of acetylcholinesterase. Mol Cell Biol 19, 788-795.
Hammond, S. M., Boettcher, S., Caudy, A. A., Kobayashi, R., and Hannon, G. J. (2001). Argonaute2, a link between genetic and biochemical analyses of RNAi. Science 293, 1146-1150.
Hannon, G. J. (2002). RNA interference. Nature 418, 244-251.
Holen, T., Amarzguioui, M., Wiiger, M. T., Babaie, E., and Prydz, H. (2002). Positional effects of short interfering RNAs targeting the human coagulation trigger Tissue Factor. Nucleic Acids Res 30, 1757-1766.
Hutvagner, G., McLachlan, J., Pasquinelli, A. E., Balint, E., Tuschl, T., and Zamore, P. D. (2001). A cellular function for the RNA-interference enzyme Dicer in the maturation of the let-7 small temporal RNA. Science 293, 834-838.
Jackson, A. L., Bartz, S. R., Schelter, J., Kobayashi, S. V., Burchard, J., Mao, M., Li, B., Cavet, G., and Linsley, P. S. (2003). Expression profiling reveals off-target gene regulation by RNAi. Nat Biotechnol 21, 635-637.
Jones-Rhoades, M. W. and Bartel, D. P. (2004). Computational identification of plant micro-RNAs and their targets, including a stress-induced micro-RNA. Mol. Cell. 14(6):787-99.
Kaufer, D., Friedman, A., Seidman, S., and Soreq, H. (1999). Anticholinesterases induce multigenic transcriptional feedback response suppressing cholinergic neurotransmission. Chem Biol Interact 119-120, 349-360.
Kawasaki, H. and Taira, K. (2003a). HES1 is a target of micro-RNA-23 during retinoic-acid-induced neuronal differentiation of NT2 cells. Nature 423, 838-842.
Kawasaki, H. and Taira, K. (2003b). Retraction: HES1 is a target of micro-RNA-23 during retinoic-acid-induced neuronal differentiation of NT2 cells. Nature 426, 100.
Ketting, R. F., Fischer, S. E., Bernstein, E., Sijen, T., Hannon, G. J., and Plasterk, R. H. (2001). Dicer functions in RNA interference and in synthesis of small RNA involved in developmental timing in C. elegans. Genes Dev 15, 2654-2659.
Krieg, A. M. (2000). The role of CpG motifs in innate immunity. Curr Opin Immunol 12, 35-43.
Lassus, P., Rodriguez, J., and Lazebnik, Y. (2002). Confirming specificity of RNAi in mammalian cells. Sci STKE 2002, PL13.
Leicht, M., Greipel, N., and Zimmer, H. (2000). Comitogenic effect of catecholamines on rat cardiac fibroblasts in culture. Cardiovasc Res 48, 274-284.
Lev-Lehman, E., Deutsch, V., Eldor, A., and Soreq, H. (1997). Immature human megakaryocytes produce nuclear-associated acetylcholinesterase. Blood 89, 3644-3653.
Liebhaber, S. A. (1997). mRNA stability and the control of gene expression. Nucleic Acids Symp Ser, 36, 29-32.
Lin, S. L. and Ying, S. Y. (2004a). New drug design for gene therapy—taking advantage of introns. Letters Drug Des & Discovery 1, 256-262(7).
Lin, S. L. and Ying, S. Y. (2004b). Novel RNAi therapy—intron-derived micro-RNA drugs. Drug Des Rev—Online 1, 247-255(9).
Linette, G. P. and Korsmeyer, S. J. (1994). Differentiation and cell death: lessons from the immune system. Curr Opin Cell Biol 6, 809-815.
Lock, R. B., Liem, N., Farnsworth, M. L., Milross, C. G., Xue, C., Tajbakhsh, M., Haber, M., Norris, M. D., Marshall, G. M., and Rice, A. M. (2002). The nonobese diabetic/severe combined immunodeficient (NOD/SCID) mouse model of childhood acute lymphoblastic leukemia reveals intrinsic differences in biologic characteristics at diagnosis and relapse. Blood 99, 4100-4108.
Lumkul, R., Gorin, N. C., Malehom, M. T., Hoehn, G. T., Zheng, R., Baldwin, B., Small, D., Gore, S., Smith, D., Meltzer, P. S., and Civin, C. I. (2002). Human AML cells in NOD/SCID mice: engraftment potential and gene expression. Leukemia 16, 1818-1826.
Ma, F., Manabe, A., Wang, D., Ito, M., Kikuchi, A., Wada, M., Ohara, A., Hosoya, R., Asano, S., and Tsuji, K. (2002). Growth of human T cell acute lymphoblastic leukemia lymphoblasts in NOD/SCID mouse fetal thymus organ culture. Leukemia 16, 1541-1548.
Macfarlane, D. E., Manzel, L., and Krieg, A. M. (1997). Unmethylated CpG-containing oligodeoxynucleotides inhibit apoptosis in WEHI 231 B lymphocytes induced by several agents: evidence for blockade of apoptosis at a distal signalling step. Immunology 91, 586-593.
Martinez, J., Patkaniowska, A., Urlaub, H., Luhrmann, R., and Tuschl, T. (2002). Single-stranded antisense siRNAs guide target RNA cleavage in RNAi. Cell 110, 563-574.
Meshorer, E., Erb, C., Gazit, R., Pavlovsky, L., Kaufer, D., Friedman, A., Glick, D., Ben-Arie, N., and Soreq, H. (2002). Alternative splicing and neuritic mRNA translocation under long-term neuronal hypersensitivity. Science 295, 508-512.
Nicholson, R. H. and Nicholson, A. W. (2002). Molecular characterization of a mouse cDNA encoding Dicer, a ribonuclease III ortholog involved in RNA interference. Mamm Genome 13, 67-73.
Nijmeijer, B. A., Willemze, R., and Falkenburg, J. H. (2002). An animal model for human cellular immunotherapy: specific eradication of human acute lymphoblastic leukemia by cytotoxic T lymphocytes in NOD/scid mice. Blood 100, 654-660.
Nishikura, K. (2001). A short primer on RNAi: RNA-directed RNA polymerase acts as a key catalyst. Cell 107, 415-418.
Nunez, R. (2001). DNA measurement and cell cycle analysis by flow cytometry. Curr Issues Mol Biol 3, 67-70.
Opalinska, J. B. and Gewirtz, A. M. (2002). Nucleic-acid therapeutics: basic principles and recent applications. Nat Rev Drug Discov 1, 503-514.
Paddison, P. J., Caudy, A. A., Bernstein, E., Hannon, G. J., and Conklin, D. S. (2002). Short hairpin RNAs (shRNAs) induce sequence-specific silencing in mammalian cells. Genes Dev 16, 948-958.
Patinkin, D., Seidman, S., Eckstein, F., Benseler, F., Zakut, H., and Soreq, H. (1990). Manipulations of cholinesterase gene expression modulate murine megakaryocytopoiesis in vitro. Mol Cell Biol 10, 6046-6050.
Perry, C., Eldor, A., and Soreq, H. (2002a). Runx1/AML1 in leukemia: disrupted association with diverse protein partners. Leuk Res 26, 221-228.
Perry, C., Sklan, E. H., and Soreq, H. (2004). CREB regulates ACHE-R-induced proliferation of human glioblastoma cells. Neoplasia, 6, 279-286.
Perry, C., Sklan, E. H., Birikh, K., Shapira, M., Trejo, L., Eldor, A., and Soreq, H. (2002b). Complex regulation of acetylcholinesterase gene expression in human brain tumors. Oncogene 21, 8428-8441.
Perry, C. and Soreq, H. (2002). Transcriptional regulation of erythropoiesis. Fine tuning of combinatorial multi-domain elements. Eur J Biochem 269, 3607-3618.
Pick, M. (2004) PhD Thesis. “Hematopoietic roles of acetylcholinesterase and its variant C-terminal peptides.” The Hebrew University of Jerusalem. Pick M, Flores-Flores C, Grisaru D, Deutsch V, Soreq H. Blood-cell Specific Acetylcholinesterase Splice Variations under Changing Stimuli. Annals of New York Academy of Science. 2004; 1018:85-95.
Pilarski, L. M., Seeberger, K., Coupland, R. W., Eshpeter, A., Keats, J. J., Taylor, B. J., and Belch, A. R. (2002). Leukemic B cells clonally identical to myeloma plasma cells are myelomagenic in NOD/SCID mice. Exp Hematol 30, 221-228.
Prohaska, S. S., Scherer, D. C., Weissman, I. L., and Kondo, M. (2002). Developmental plasticity of lymphoid progenitors. Semin Immunol 14, 377-384.
Rao, R. V., Hermel, E., Castro-Obregon, S., del Rio, G., Ellerby, L. M., Ellerby, H. M., and Bredesen, D. E. (2001). Coupling endoplasmic reticulum stress to the cell death program. Mechanism of caspase activation. J Biol Chem. 276(36), 33869-33874.
Ratajczak, J., Kijowski, J., Majka, M., Jankowski, K., Reca, R., and Ratajczak, M. Z. (2003). Biological significance of the different erythropoietic factors secreted by normal human early erythroid cells. Leuk Lymphoma 44, 767-774.
Razon, N., Soreq, H., Roth, E., Bartal, A., and Silman, I. (1984). Characterization of activities and forms of cholinesterases in human primary brain tumors. Exp Neurol 84, 681-695.
Rosenberg, D., Groussin, L., Jullian, E., Perlemoine, K., Bertagna, X., and Bertherat, J. (2002). Role of the PKA-regulated transcription factor CREB in development and tumorigenesis of endocrine tissues. Ann N Y Acad Sci 968, 65-74.
Scanlon, K. J., Ohta, Y., Ishida, H., Kijima, H., Ohkawa, T., Kaminski, A., Tsai, J., Horng, G., and Kashani-Sabet, M. (1995). Oligonucleotide-mediated modulation of mammalian gene expression. FASEB J 9, 1288-1296.
Schemesta, M., Pfeffer, P. L., and Busslinger, M. (2002). Control of pre-BCR signaling by Pax5-dependent activation of the BLNK gene. Immunity 17, 473-485.
Schweighoffer, E., Vanes, L., Mathiot, A., Nakamura, T., and Tybulewicz, V. L. (2003). Unexpected requirement for ZAP-70 in pre-B cell development and allelic exclusion. Immunity 18, 523-533.
Shapira, M., Grant, A., Korner, M., and Soreq, H. (2000). Genomic and transcriptional characterization of the human AChE locus: complex involvement with acquired and inherited diseases. Isr Med Assoc J 2, 470-473.
Sharp, P. A. (1999). RNAi and double-strand RNA. Genes Dev 13, 139-141.
Soreq, H., Nudel, U., Salomon, R., Revel, M. and Littauer, U. Z. (1974) In vitro translation of polyadenylic acid-free rabbit globin messenger RNA. J. Mol. Biol. 88, 233-245.
Soreq, H., Ehrlich, G., Schwarz, M., Loewenstein, Y., Glick, D., and Zakut, H. (1994). Mutations and impaired expression in the ACHE and BCHE genes: neurological implications. Biomed Pharmacother 48(5-6), 253-9.
Soreq, H. and Seidman, S. (2001). Acetylcholinesterase—new roles for an old actor. Nature Reviews Neuroscience 2, 294-302.
Stein, C. A. (1998). How to design an antisense oligodeoxynucleotide experiment: a consensus approach. Antisense Nucleic Acid Drug Dev 8, 129-132.
Steinman, R. A. (2002). Cell cycle regulators and hematopoiesis. Oncogene 21, 3403-3413.
Sui, G., Soohoo, C., Affar, E. B., Gay, F., Shi, Y., and Forrester, W. C. (2002). A DNA vector-based RNAi technology to suppress gene expression in mammalian cells. Proc Natl Acad Sci USA 99, 5515-5520.
Takasuka, N., White, M. R. H., Wood, C. D., Robertson, W. R., and Davis, J. R. E. (1998). Dynamic changes in prolactin promoter activation in individual living lactotrophic cells. Endocrinology 139, 1361-1368.
Thastrup, O., Cullen, P. J., Drobak, B. K., Hanley, M. R., and Dawson, A. P. (1990). Thapsigargin, a tumor promoter, discharges intracellular Ca²⁺ stores by specific inhibition of the endoplasmic reticulum Ca²⁺-ATPase. Proc Natl Acad Sci USA 87, 2466-2470.
Thisted, T., Lyakhov, D. L., and Liebhaber, S. A. (2001). Optimized RNA targets of two closely related triple KH domain proteins, heterogeneous nuclear ribonucleoprotein K and alphaCP-2KL, suggest Distinct modes of RNA recognition. J Biol Chem 276, 17484-17496.
Tracey, K. J. (2002). The inflammatory reflex. Nature 420, 853-859.
Vaucheret, H., Vazquez, F., Crete, P., and Bartel, D. P. (2004). The action of ARGONAUTE1 in the micro-RNA pathway and its regulation by the micro-RNA pathway are crucial for plant development. Genes Dev. 18(10), 1187-1197.
Waelti, E. R. and Gluck, R. (1998). Delivery to cancer cells of antisense L-myc oligonucleotides incorporated in fusogenic, cationic-lipid-reconstituted influenza-virus envelopes (cationic virosomes). Int J Cancer 77, 728-733.
Wang, X., Kiledjian, M., Weiss, I. M., and Liebhaber, S. A. (1995). Detection and characterization of a 3′ untranslated region ribonucleoprotein complex associated with human alpha-globin mRNA stability. Mol Cell Biol 15, 1769-1777.
Weiss, I. M. and Liebhaber, S. A. (1995). Erythroid cell-specific mRNA stability elements in the alpha 2-globin 3′ nontranslated region. Mol Cell Biol 15, 2457-2465.
Williams, R. W. and Rubin, G. M. (2002). ARGONAUTE1 is required for efficient RNA interference in Drosophila embryos. Proc Natl Acad Sci USA 99, 6889-6894.
Yang, D., Lu, H., and Erickson, J. W. (2000). Evidence that processed small dsRNAs may mediate sequence-specific mRNA degradation during RNAi in Drosophila embryos. Curr Biol 10, 1191-1200.
Yang, S., Tutton, S., Pierce, E., and Yoon, K. (2001). Specific double-stranded RNA interference in undifferentiated mouse embryonic stem cells. Mol Cell Biol 21, 7807-7816.
Yu, J. Y., DeRuiter, S. L., and Turner, D. L. (2002). RNA interference by expression of short-interfering RNAs and, hairpin RNAs in mammalian cells. Proc Natl Acad Sci USA 99, 6047-6052.
Zamore, P. D., Tuschl, T., Sharp, P. A., and Bartel, D. P. (2000). RNAi: double-stranded RNA directs the ATP-dependent cleavage of mRNA at 21 to 23 nucleotide intervals. Cell 101, 25-33.

What is claimed is:

1. A method of regulating an AChE-associated biological pathway having a miRNA component, the method comprising subjecting the AChE-associated biological pathway to an agent capable of regulating a function of the miRNA, thereby regulating the AChE-associated biological pathway.

2. The method of claim 1, wherein said agent is a polynucleotide.

3. The method of claim 2, wherein said polynucleotide is selected from the group consisting of a polynucleotide which comprises at least 10 consecutive nucleotides of the nucleic acid sequence set forth in SEQ ID NO:1, a polynucleotide hybridizable in cells under physiological conditions to an RNA molecule which comprises a nucleic acid sequence as set forth in SEQ ID NO:2, a polynucleotide as set forth by SEQ ID NO:1, a polynucleotide which comprises at least 10 consecutive nucleotides of the nucleic acid sequence set forth in SEQ ID NO:2, a polynucleotide hybridizable in cells under physiological conditions to an RNA molecule which comprises a nucleic acid sequence as set forth in SEQ ID NO:21 and/or 22, a polynucleotide as set forth by SEQ ID NO:2, a polynucleotide which comprises at least 25 consecutive nucleotides of the nucleic acid sequence set forth in SEQ ID NO:13, a polynucleotide as set forth by SEQ ID NO:13, a polynucleotide which comprises at least 20 consecutive nucleotides of SEQ ID NO:13 and/or at least 10 consecutive nucleotides of SEQ ID NO:1, a polynucleotide as set forth by SEQ ID NO:12 or a functional homolog thereof, a polynucleotide as set forth by SEQ ID: 19 or a functional homolog thereof, a polynucleotide as set forth by SEQ ID NO:23, a polynucleotide as set forth by SEQ ID NO: 24, a polynucleotide as set forth by SEQ ID NO:107, a polynucleotide as set forth by SEQ ID NO:108, a polynucleotide as set forth by SEQ ID NO:109 and a polynucleotide as set forth by SEQ ID NO:110.

4. The method of claim 1, wherein said miRNA is set forth by the sequence selected from the group consisting of SEQ ID NOs: 54, 93, 94, 98, 99, 100, 21 and 22.

5. A method of regulating an expression level ratio of ACHE-S and ACHE-R and/or AChE-S mRNA and AChE-R mRNA splice variants in AChE expressing cells comprising subjecting the ACHE gene expressing cells to an agent capable of regulating a function of a miRNA component associated with regulating the expression level ratio of AChE-S and AChE-R splice variants, thereby regulating the expression level of the ACHE-S and ACHE-R splice variants in the AChE expressing cells.

6. The method of claim 5, wherein said agent is a polynucleotide.

7. The method of claim 6, wherein said polynucleotide is selected from the group consisting of a polynucleotide which comprises at least 10 consecutive nucleotides of the nucleic acid sequence set forth in SEQ ID NO:1, a polynucleotide hybridizable in cells under physiological conditions to an RNA molecule which comprises a nucleic acid sequence as set forth in SEQ ID NO:2, a polynucleotide as set forth by SEQ ID NO:1, a polynucleotide which comprises at least 10 consecutive nucleotides of the nucleic acid sequence set forth in SEQ ID NO:2, a polynucleotide hybridizable in cells under physiological conditions to an RNA molecule which comprises a nucleic acid sequence as set forth in SEQ ID NO:21 and/or 22, a polynucleotide as set forth by SEQ ID NO:2, a polynucleotide which comprises at least 25 consecutive nucleotides of the nucleic acid sequence set forth in SEQ ID NO:13, a polynucleotide as set forth by SEQ ID NO:13, a polynucleotide which comprises at least 20 consecutive nucleotides of SEQ ID NO:13 and/or at least 10 consecutive nucleotides of SEQ ID NO:1, a polynucleotide as set forth by SEQ ID NO:12 or a functional homolog thereof, a polynucleotide as set forth by SEQ ID: 19 or a functional homolog thereof, a polynucleotide as set forth by SEQ ID NO:23 and a polynucleotide as set forth by SEQ ID NO: 24.

8. The method of claim 4, wherein said miRNA is set forth by the sequence selected from the group consisting of SEQ ID NOs: 54, 93, 94, 98, 99, 100, 21, 22 and 107 and 108.

9. A method of treating a pathology related to an AChE-associated biological pathway, the method comprising administering to a subject in need thereof an agent capable of regulating a function of a miRNA component of the AChE-associated biological pathway, thereby treating the pathology.

10. The method of claim 9, wherein the pathology is a disease or condition in which regulating nitric oxide levels is therapeutically beneficial.

11. The method of claim 9, wherein the pathology is associated with abnormal levels of ACHE-S or ACHE-R splice variants.

12. The method of claim 9, wherein said polynucleotide is selected from the group consisting of a polynucleotide which comprises at least 10 consecutive nucleotides of the nucleic acid sequence set forth in SEQ ID NO:1, a polynucleotide hybridizable in cells under physiological conditions to an RNA molecule which comprises a nucleic acid sequence as set forth in SEQ ID NO:2, a polynucleotide as set forth by SEQ ID NO:1, a polynucleotide which comprises at least 10 consecutive nucleotides of the nucleic acid sequence set forth in SEQ ID NO:2, a polynucleotide hybridizable in cells under physiological conditions to an RNA molecule which comprises a nucleic acid sequence as set forth in SEQ ID NO:21 and/or 22, a polynucleotide as set forth by SEQ ID NO:2, a polynucleotide which comprises at least 25 consecutive nucleotides of the nucleic acid sequence set forth in SEQ ID NO:13, a polynucleotide as set forth by SEQ ID NO:13, a polynucleotide which comprises at least 20 consecutive nucleotides of SEQ ID NO:13 and/or at least 10 consecutive nucleotides of SEQ ID NO:1, a polynucleotide as set forth by SEQ ID NO:12 or a functional homolog thereof, a polynucleotide as set forth by SEQ ID: 19 or a functional homolog thereof, a polynucleotide as set forth by SEQ ID NO:23 and a polynucleotide as set forth by SEQ ID NO: 24.

13. The method of claim 9, wherein said miRNA is set forth by the sequence selected from the group consisting of SEQ ID NOs: 54, 93, 94, 98, 99, 100, 21, 22, 107 and 108.

14. A method of altering differentiation and/or proliferation of hematopoietic progenitor and/or stem cells, the method comprising subjecting the progenitor and/or stem cells to an agent capable of regulating a function a miRNA component of an AChE-associated biological pathway in the progenitor and/or stem cells, thereby altering differentiation and/or proliferation of the hematopoietic progenitor and/or stem cells.

15. A method of regulating apoptosis in cells and/or a tissue of a subject in need thereof, the method comprising subjecting the cells and/or the tissue of the subject to an agent capable of regulating a function a miRNA component of an AChE-associated biological pathway in the cells and/or tissue, thereby regulating apoptosis in the cells and/or the tissue of the subject.

16. The method of claim 15, wherein said agent is a polynucleotide.

17. The method of claim 16, wherein said polynucleotide is selected from the group consisting of a polynucleotide which comprises at least 10 consecutive nucleotides of the nucleic acid sequence set forth in SEQ ID NO:1, a polynucleotide hybridizable in cells under physiological conditions to an RNA molecule which comprises a nucleic acid sequence as set forth in SEQ ID NO:2, a polynucleotide as set forth by SEQ ID NO:1, a polynucleotide which comprises at least 10 consecutive nucleotides of the nucleic acid sequence set forth in SEQ ID NO:2, a polynucleotide hybridizable in cells under physiological conditions to an RNA molecule which comprises a nucleic acid sequence as set forth in SEQ ID NO:21 and/or 22, a polynucleotide as set forth by SEQ ID NO:2, a polynucleotide which comprises at least 25 consecutive nucleotides of the nucleic acid sequence set forth in SEQ ID NO:13, a polynucleotide as set forth by SEQ ID NO:13, a polynucleotide which comprises at least 20 consecutive nucleotides of SEQ ID NO:13 and/or at least 10 consecutive nucleotides of SEQ ID NO:1, a polynucleotide as set forth by SEQ ID NO:12 or a functional homolog thereof, a polynucleotide as set forth by SEQ ID: 19 or a functional homolog thereof, a polynucleotide as set forth by SEQ ID NO:23 and a polynucleotide as set forth by SEQ ID NO: 24.

18. The method of claim 15, wherein said miRNA is set forth by the sequence selected from the group consisting of SEQ ID NOs: 54, 93, 94, 98, 99, 100, 21, 22, 107 and 108.

19. A method of diagnosing a pathology associated with abnormal function of a miRNA component of an AChE-associated biological pathway in a subject, the method comprising obtaining a biological sample from the subject and determining a level of the miRNA in cells of said biological sample, wherein a level of the miRNA above or below a predetermined threshold or range is indicative of a presence of a pathology associated with abnormal function of the miRNA.

20. The method of claim 19, wherein said miRNA component is set forth by SEQ NO:21 or SEQ ID NO: 22.

21. The method of claim 19, wherein said miRNA component is set forth by a sequence selected from the group consisting of SEQ ID NOs: 54, 93, 94, 98, 99 and 100.

22. The method of claim 19, wherein said determining is effected using an oligonucleotide.

23. The method of claim 22, wherein said oligonucleotide specifically hybridizable with said miRNA under stringent hybridization conditions.

24. The method of claim 22, wherein said oligonucleotide is capable of specifically hybridizing with a polynucleotide having a nucleic acid sequence as set forth by SEQ ID NO:21 and/or 22 under stringent hybridization conditions.

25. The method of claim 19, wherein said determining is effected using at least one oligonucleotide capable of specifically amplifying a polynucleotide having a nucleic acid sequence as set forth in SEQ ID NO:21 and/or 22.

26. The method of claim 19, wherein said biological sample is selected from the group consisting of blood, bone marrow, spinal fluid and cord blood.

27. An isolated polynucleotide comprising a nucleic acid sequence which comprises at least 10 consecutive nucleotides of the nucleotide sequence set forth in SEQ ID NO:1, with the proviso that isolated polynucleotide is not identical to the sequence set forth in SEQ ID NO:1.

28. An isolated polynucleotide comprising a nucleic acid sequence of 10-50 bases and capable of hybridizing in cells under physiological conditions with an RNA molecule which comprises a nucleotide sequence as set forth in SEQ ID NO:2, with the proviso that isolated polynucleotide is not identical to the sequence set forth in SEQ ID NO:1.

29. The isolated polynucleotide of claim 27, wherein the polynucleotide is a modified polynucleotide.

30. The isolated polypeptide of claim 27, as set forth by SEQ ID NO:23 or 24.

31. A pharmaceutical composition comprising as an active ingredient the polynucleotide of claim 27 and a pharmaceutically acceptable carrier.

32. An isolated polynucleotide comprising a nucleic acid sequence which comprises at least 10 consecutive nucleotides from the nucleotide sequence set forth by SEQ ID NO:2.

33. An isolated polynucleotide comprising a nucleic acid sequence of 10-50 bases and capable of hybridizing in cells under physiological conditions with an RNA molecule which comprises a nucleotide sequence as set forth in SEQ ID NO:1.

34. The isolated polynucleotide of claim 33, wherein said nucleic acid sequence is as set forth in SEQ ID NO:2.

35. The isolated polynucleotide of claim 33, wherein the polynucleotide is a modified polynucleotide.

36. The isolated polynucleotide of claim 35, wherein said modified polynucleotide is set forth by SEQ ID NO:23, 24, 107, 108, 109 or 110.

37. A pharmaceutical composition comprising, as an active ingredient the polynucleotide of claim 33 and a pharmaceutically acceptable carrier.

38. An isolated polynucleotide comprising a nucleic acid sequence which comprises at least 20 consecutive nucleotides from the nucleotide sequence set forth in SEQ ID NO:13, with the proviso that isolated polynucleotide is not identical to the sequence set forth in SEQ ID NO:13.

39. The isolated polynucleotide of claim 38, wherein the polynucleotide is a modified polynucleotide.

40. The isolated polypeptide of claim 39, as set forth by SEQ ID NO:23 or 24.

41. A pharmaceutical composition comprising, as an active ingredient, the polynucleotide of claim 38 and a pharmaceutically acceptable carrier.

42. An isolated polynucleotide as set forth in SEQ ID NO:107, 108, 109 or 110.

43. A pharmaceutical composition comprising as an active ingredient a polynucleotide as set forth in SEQ ID NO:107, 108, 109 or 110.
